(19) United States
(12) Patent Application Publication
(10) Pub. No.: US 2008/0241835 A1
(43) Pub. Date: Oct. 2, 2008

(54) DIFFERENTIALLY EXPRESSED GENES INVOLVED IN ANGIOGENESIS, THE POLYPEPTIDES ENCODED THEREBY, AND METHODS OF USING THE SAME

(75) Inventors: Fuad Mehraban, Trumbull, CT (US); Mary Gerritsen, San Mateo, CA (US); Luca Rastelli, Guilford, CT (US)

Correspondence Address: MERCHANT & GOULD PC, P.O. BOX 2903, MINNEAPOLIS, MN, 55402-0903, US

(73) Assignees: Genentech, Inc., South San Francisco, CA (US); CuraGen Corp., New Haven, CT (US)

(21) Appl. No.: 11/927,609

(22) Filed: Oct. 29, 2007

Related U.S. Application Data
(62) Division of application No. 09/703,350, filed on Oct. 31, 2000.
(60) Provisional application No. 60/162,699, filed on Nov. 1, 1999.
(60) Provisional application No. 60/196,802, filed on Apr. 13, 2000.

(51) Int. Cl. C12Q 1/68; C12Q 1/00

(52) U.S. Cl. 435/6; 435/4

(57) ABSTRACT

The present invention is directed to nucleic acid sequences and the polypeptides encoded thereby that are differentially expressed in angiogenesis. Also provided are methods for stimulating or inhibiting angiogenesis in mammals, including humans. Pharmaceutical compositions based on polypeptides, agonists, or antagonists thereto are also provided. Additionally, the invention also provides methods for diagnosing and treating angiogenic disorders including, but not limited to, wound healing and cancer.

### RELATED APPLICATIONS

This application claims priority to U.S. Ser. No. 60/162,699, filed Nov. 1, 1999 and U.S. Ser. No. 60/196802, filed Apr. 13, 2000, both of which are incorporated herein by reference.

### FIELD OF THE INVENTION

The invention relates generally to the identification of nucleic acids and their encoded intracellular polypeptides, whose expression is modulated in cells undergoing angiogenesis and/or vascularization. These nucleic acids and proteins have not previously been identified as having a biological role in the process of angiogenesis or endothelial cell differentiation into tube-like structure. The invention further relates to methods useful for promoting or inhibiting angiogenesis and/or cardiovascularization in mammals in need of such biological effect. This includes the diagnosis and treatment of cardiovascular disorders as well as oncological disorders. Additionally, the present invention further relates to the use of anti-PA polypeptide antibodies as diagnostic probes or as therapeutic agents as well as the use of polynucleotide sequences encoding PA polypeptides as diagnostic probes or therapeutic agents.

### BACKGROUND OF THE INVENTION

Intracellular proteins play important roles in, among other things, the formation, differentiation and maintenance of multicellular organisms. The fate of many individual cells, e.g., proliferation, migration, differentiation, or interaction with other cells, is typically governed by information received from other cells and/or the immediate environment. This information is often transmitted via secreted polypeptides (for instance, mitogenic factors, survival factors, cytotoxic factors, differentiation factors, neuropeptides, and hormones) which are, in turn, recognized by and activate diverse cell receptors or membrane-bound proteins. Each activation signal initiates a specific, signal transduction pathway composed of intracellular proteins (e.g., protein kinases, DNA-binding regulatory proteins, protein processing proteins, proteases, glycosidases) resulting in the modulation, either up- or down-regulation, of the activity, expression, or amount of other intracellular proteins involved in or necessary for the cell's fate in response to the signal. For example, detectable changes in the RNA or protein levels of intracellular proteins necessary for cell growth or differentiation in response to transduction of signals that regulate cell growth and differentiation can be controlled in part by receptor-mediated phosphorylation of signal-induction-pathway related intracellular proteins.

Intracellular proteins and their gene sequences have various industrial applications, including as drug targets for pharmaceuticals, diagnostics, pharmaceuticals, biosensors, and bioreactors. While most protein drugs available at present are secreted cytokines or their antibody mimics, most targets of small molecule, peptide, or antisense drugs are intracellular proteins, or the intracellular genes that encode them. For example, such drugs can interact with an intracellular protein target to block its activity and disrupt the related signal transduction pathway, thereby stopping (or modulating) the cell's response or activity controlled by that pathway. Efforts are underway to identify new, native intracellular proteins and their genes, the signal transduction pathways in which they function, and the intracellular proteins or genes they modulate. Such genes and their proteins are typically discovered by binary comparison studies in which a differential analysis is made of RNA or protein upon a cell or tissue response to a certain stimuli.

There exists a need for additional products, methods and assays that provide a means to control angiogenesis or modulate cellular responses to angiogenic stimuli and tissue response to such stimuli. Such products, methods and assays will provide benefit in numerous medical conditions and procedures.

### SUMMARY OF THE INVENTION

In one aspect, the invention involves a method of assessing the efficacy of an angiogenic disorder treatment in a subject, wherein the method involves the steps of providing a test cell population capable of expressing one or more of the PA:1-27 nucleic acid sequences; detecting the expression of one or more of these nucleic acid sequences; comparing the expression to that of the nucleic acid sequences in a reference cell population whose angiogenic stage is known; and identifying a difference in expression level, if present, between the test cell population and the reference cell population. In various embodiments, the subject can be a mammal, or, more preferably, a human. In other embodiments, the test cell population can be provided in vitro, ex vivo from a mammalian subject, or in vivo in a mammalian subject. The expression of the nucleic acid sequences may be either increased or decreased in the test cell population as compared to the reference cell population.

In a further aspect, the invention involves a method of diagnosing an angiogenic disorder, wherein the method involves the steps of providing a test cell population capable of expressing one or more of the PA:1-27 nucleic acid sequences; detecting the expression of one or more of these nucleic acid sequences; comparing the expression to that of the nucleic acid sequences in a reference cell population whose angiogenic stage is known; and identifying a difference in expression level, if present, between the test cell population and the reference cell population. In various embodiments, the subject can be a mammal, or, more preferably, a human. In other embodiments, the test cell population can be provided in vitro, ex vivo from a mammalian subject, or in vivo in a mammalian subject. The expression of the nucleic acid sequences may be either increased or decreased in the test cell population as compared to the reference cell population.

In another aspect, the invention involves a method of identifying a test therapeutic agent for treating an angiogenic disorder in a subject involving the steps of providing a test cell population capable of expressing one or more of the PA:1-27 nucleic acid sequences; contacting the test cell population with the test therapeutic agent; detecting the expression of one or more of these nucleic acid sequences; comparing the expression to that of the nucleic acid sequences in a reference cell population whose angiogenic stage is known; and identifying a difference in expression level, if present, between the test cell population and the reference cell population. In different embodiments, the subject may be a mammal or, more preferably, a human. Additionally, the test therapeutic agent may be either a known anti-angiogenic disorder agent or an unknown anti-angiogenic disorder agent. When the test therapeutic agent is a know anti-angiogenic disorder agent, be an agonist or an antagonist of a native PA polypeptide. The agonist may be an anti-PA antibody. Likewise, the antagonist may also be an anti-PA antibody. The angiogenic disorder to be treated can be selected from the following diseases or disorders: vascular tumors, proliferative vitreoretinopathy, rheumatoid arthritis, Crohn's disease, atherosclerosis, ovarian hyperstimulation, psoriasis, endometriosis associated with neovascularization, restenosis subsequent to balloon angioplasty, scar tissue overproduction, peripheral vascular disease, hypertension, inflammatory vasculitides, Reynaud's disease and Reynaud's phenomenon, aneurysms, arterial restenosis, thrombophlebitis, lymphangitis, lymphedema, wound healing and tissue repair, ischemia reperfusion injury, angina, myocardial infarctions, chronic heart conditions, heart failure such as congestive heart failure, age-related macular degeneration, and osteoporosis.

In a further aspect, the invention involves a method of identifying or determining the susceptibility to an angiogenic disorder in a subject. In this aspect, the method involves the steps of providing a test cell population capable of expressing one or more of the PA:1-27 nucleic acid sequences; detecting the expression of one or more of these nucleic acid sequences; comparing the expression to that of the nucleic acid sequences in a reference cell population whose angiogenic stage is known; and identifying a difference in expression level, if present, between the test cell population and the reference cell population. The subject may be a mammal, or, more preferably, a human.

In an alternative aspect, the invention involves a method of treating an angiogenic disorder by administering an agent that modulates the expression or activity of one or more of the PA:1-27 nucleic acid sequences to a patient suffering from or at risk for developing the angiogenic disorder. This agent can be one that decreases the expression of one or more of PA:5, 14, and 15. Alternatively, it can be one that increases the expression of one or more of PA:1-4, 6-13, and 16-26. Additionally, the agent can be an antibody to a polypeptide encoded by the PA nucleic acid sequence, an antisense nucleic acid molecule, a peptide, a PA polypeptide agonist, a PA polypeptide antagonist, a peptidomimetic, a small molecule, or another drug.

The invention also includes a kit containing one or more reagents for detecting two or more of the PA:1-27 nucleic acid sequences. Additionally, the invention involves an array of probe nucleic acids capable of detected two or more of the PA:1-27 nucleic acids.

The polypeptides and nucleic acids of the invention can be used to treat an angiogenic disorder in a subject. Treatment of an angiogenic disorder may be in a mammal, preferably a human. In various embodiments, therapeutic compositions containing the polypeptides and nucleic acids of the invention can be used to treat angiogenic disorders. These therapeutic compositions can include a pharmaceutically acceptable carrier and, additionally, an active ingredient such as a cardiovascular agent, an endothelial agent, an angiogenic agent, or an angiostatic agent. Also provided is a kit containing a therapeutic composition for use in the treatment of an angiogenic disorder along with a pharmaceutically acceptable carrier, wherein the therapeutic composition is a PA polypeptide, an agonist of a PA polypeptide, or an antagonist of a PA polypeptide.

In another aspect, this invention involves an isolated polypeptide that is at least 80% identical to a polypeptide having the sequence of SEQ ID NO:72, or fragments, derivatives, analogs, or homologs thereof. Additionally, the invention also involves an antibody to the polypeptide, fragment, derivative, analog, and/or homolog.

Also included in the invention is an isolated nucleic acid molecule that this at least 75% identical to the nucleic acid encoding the polypeptide of SEQ ID NO:72, or the complement of the nucleic acid sequence, as well as vectors and host cells containing this nucleic acid sequence.

In still further aspects, the invention involves pharmaceutical compositions containing either the isolated nucleic acid or the isolated polypeptide. Another aspect involves methods of detecting the presence of the nucleic acid and polypeptide.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and are not intended to be limiting.

Other features and advantages of the invention will be apparent from the following detailed description and from the claims.

### BRIEF DESCRIPTION OF THE DRAWINGS

FIG. 1 shows photomicrographs of hHUVECs grown in a collagen gel matrix (panels A-I) and when plated on a collagen film (panel J).

FIG. 2 shows a schematic presentation of the various steps involved in endothelial cell differentiation into a tube-like structure.

FIG. 3 is a diagram showing the TaqMan PCR analysis of time course of expression of osteonidogen in a collagen gel (light shaded bars) and on a collagen film (dark shaded bars).

FIG. 4 is a diagram showing the TaqMan PCR analysis of time course of expression of laminin gamma-2 chain (nicein) in a collagen gel (light shaded bars) and on a collagen film (dark shaded bars).

FIG. 5 is a diagram showing the TaqMan PCR analysis of time course of expression of podocalyxin in a collagen gel (light shaded bars) and on a collagen film (dark shaded bars).

FIG. 6 is a diagram showing the TaqMan PCR analysis of time course of expression of moesin in a collagen gel (light shaded bars) and on a collagen film (dark shaded bars).

FIG. 7 is a diagram showing the TaqMan PCR analysis of time course of expression of mesoendothelial keratin in a collagen gel (light shaded bars) and on a collagen film (dark shaded bars).

FIG. 8 is a diagram showing the TaqMan PCR analysis of time course of expression of T-plastin in a collagen gel (light shaded bars) and on a collagen film (dark shaded bars).

FIG. 9 is a diagram showing the TaqMan PCR analysis of time course of expression of actin bundling protein in a collagen gel (light shaded bars) and on a collagen film (dark shaded bars).

FIG. 10 is a diagram showing the TaqMan PCR analysis of time course of expression of brain ankyrin-2 in a collagen gel (light shaded bars) and on a collagen film (dark shaded bars).

FIG. 11 is a diagram showing the TaqMan PCR analysis of time course of expression of tissue factor pathway inhibitor-2 in a collagen gel (light shaded bars) and on a collagen film (dark shaded bars).

FIG. 12 is a diagram showing the TaqMan PCR analysis of time course of expression of cathepsin B in a collagen gel (light shaded bars) and on a collagen film (dark shaded bars).

FIG. 13 is a diagram showing the TaqMan PCR analysis of time course of expression of plasminogen activator inhibitor in a collagen gel (light shaded bars) and on a collagen film (dark shaded bars).

FIG. 14 is a diagram showing the TaqMan PCR analysis of time course of expression of ADAMTS-4 in a collagen gel (light shaded bars) and on a collagen film (dark shaded bars).

FIG. 15 is a diagram showing the TaqMan PCR analysis of time course of expression of ax1 in a collagen gel (light shaded bars) and on a collagen film (dark shaded bars).

FIG. 16 is a diagram showing the TaqMan PCR analysis of time course of expression of ECK in a collagen gel (light shaded bars) and on a collagen film (dark shaded bars).

FIG. 17 is a diagram showing the TaqMan PCR analysis of time course of expression of OX-40 in a collagen gel (light shaded bars) and on a collagen film (dark shaded bars).

FIG. 18 is a diagram showing the TaqMan PCR analysis of time course of expression of gp130 in a collagen gel (light shaded bars) and on a collagen film (dark shaded bars).

FIG. 19 is a diagram showing the TaqMan PCR analysis of time course of expression of CD82 in a collagen gel (light shaded bars) and on a collagen film (dark shaded bars).

FIG. 20 is a diagram showing the TaqMan PCR analysis of time course of expression of PRZ in a collagen gel (light shaded bars) and on a collagen film (dark shaded bars).

FIG. 21 is a diagram showing the TaqMan PCR analysis of time course of expression of alpha 2 integrin in a collagen gel (light shaded bars) and on a collagen film (dark shaded bars).

FIG. 22 is a diagram showing the TaqMan PCR analysis of time course of expression of PIGF in a collagen gel (light shaded bars) and on a collagen film (dark shaded bars).

FIG. 23 is a diagram showing the TaqMan PCR analysis of time course of expression of stanniocalcin precursor in a collagen gel (light shaded bars) and on a collagen film (dark shaded bars).

FIG. 24 is a diagram showing the TaqMan PCR analysis of time course of expression of FGF 16 in a collagen gel (light shaded bars) and on a collagen film (dark shaded bars).

FIG. 25 is a diagram showing the TaqMan PCR analysis of time course of expression of White Protein Homolog in a collagen gel (light shaded bars) and on a collagen film (dark shaded bars).

FIG. 26 is a picture showing the results of haematoxylin-eosin staining (Top Panel) and fluorescence in situ hybridization (Bottom Panel) of of podocalyxin expression in vessels surrounding lung squamous cell carcinoma.

FIG. 27 is a picture showing the results of haematoxylin-eosin staining (Top Panel) and fluorescence in situ hybridization (Bottom Panel) of protein zero expression in tissue related to pulmonary adenocarcinoma.

FIG. 28 is a picture showing the results of haematoxylin-eosin staining (Top Panel) and fluorescence in situ hybridization (Bottom Panel) of stanniocalcin precursor mRNA in ductal mammary adenocarcinoma.

FIG. 29 is a picture showing the results of haematoxylin-eosin staining (Top Panel) and fluorescence in situ hybridization (Bottom Panel) of stanniocalcin precursor mRNA in squamous cell carcinoma.

FIG. 30 is the amino acid sequence of r0v0-176.7A [PA27].

FIG. 31 is a diagram showing the hydropathy plot of r0v0-176.7A [PA27].

FIG. 32 is a diagram showing the ClustalW alignment of mouse, rat and human orthologs of r0v0-176.7A [PA27].

### DETAILED DESCRIPTION OF THE INVENTION

One consequence of the generation of a cellular response to certain stimuli is the formation of new blood vessels. This can occur by two related mechanisms: (1) angiogenesis, which is the growth of new vessels from pre-existing vessels, and (2) vasculogenesis, which is the formation of vessels through aggregation of endothelial cells. The inner surfaces of all blood vessels are lined with endothelial cells. Vascular endothelial cells, located at the interface between blood and extravascular space, play prominent roles in maintaining cardiovascular homeostasis and mediating pathophysiologic responses to injury. For example, angiogenesis occurs in the adult during events such as wound healing and ovulation. During angiogenesis, endothelial cells responding to environmental stimuli undergo a number of cellular alterations and responses, resulting in a complex series of steps, which involve degradation of the basement membrane by cellular proteases, penetration and migration of endothelial cells into the extracellular matrix, endothelial proliferation, and the formation of interconnected vascular networks. This formation of new vessels takes place in distinct phases that entail and rely upon modulation or expression of a variety of intracellular proteins, extracellular matrix components, proteases and protease inhibitors, inflammatory molecules, chemokines, and molecules involved in cell division and proliferation, cytoskeletal rearrangement, adhesion molecules and also apoptosis of certain endothelial cell populations.

Endothelial cells also undergo angiogenesis during the neovascularization associated with tumor growth and metastasis as well as a variety of non-neoplastic diseases or disorders. In the case of tumor growth, angiogenesis appears to be crucial for the transition from hyperplasia to neoplasia, and for providing nourishment to the growing solid tumor (See Folkman, et al., Nature 339:58 (1989)). Angiogenesis allows tumors to be in contact with the vascular bed of the host, which, in turn, provides a route for metastasis of the tumor cells. In fact, the progression of solid tumor growth and metastasis depends on angiogenesis, as supported for example, by studies showing a correlation between the number and density of microvessels in histologic sections of invasive human breast carcinoma and actual presence of distant metastases (Weidner, et al., New Engl. J. Med., 324:1 (1991)). Recent data suggests that blocking new blood vessel growth can slow tumor growth by cutting off the supply of oxygen and nutrients. Without a new blood supply tumors cannot grow more than about 1-2 mm in diameter. Thus new angiostatic therapies to treat cancer are desired.

There exists a need for additional products, methods, and assays that provide a means to control angiogenesis or modulate cellular responses to angiogenic stimuli and tissue response to such stimuli. Such products, methods, and assays will provide benefit in numerous medical conditions and procedures.

In view of the role of vascular endothelial cell growth and angiogenesis in many diseases and disorders, it is desirable to have a means of modulating one or more of the biological effects causing these processes, in order to provide benefits such as enhancing repair or maintenance of blood vessels and reducing or inhibiting cancer and tumor progression. It is also desirable to have a means of assaying for the presence of pathogenic polypeptides in normal and diseased conditions, and especially cancer. Additionally, as there is no generally applicable therapy for the treatment of cardiac hypertrophy, the identification of factors that can prevent or reduce cardiac myocyte hypertrophy is of primary importance in the development of new therapeutic strategies to inhibit pathophysiological cardiac growth.

While there are currently several treatment modalities for various cardiovascular and oncologic disorders there is still a need for additional therapeutic approaches. The identification and characterization of novel intracellular polypeptides designated herein as “PRO-Angiogenic” polypeptides) (PA polypeptides), whose gene expression is modulated in cells undergoing angiogenesis or vasculogenesis will prove useful to meet these needs.

Several known genes and gene products have been identified in the present invention that are differentially expressed in either a positive sense or a negative sense when endothelial cells are incubated in an angiogenic, tube forming environment. A polynucleotide (or a collection of polynucleotides) and its (their) encoding polypeptide, designated in the present application as PA nucleic acid sequences and PA polypeptides, respectively, whose mRNA is modulated in endothelial cells undergoing tube formation, which is a necessary step in the development of a blood vessel during angiogenesis and vasculogenesis has been identified. Differential cDNA screening, known as GeneCalling™ technology, was applied to human umbilical cord endothelial cells (HUVECS) undergoing tube formation in collagen gels in the presence of growth factors, which mimicked the angiogenic environment of endothelial cells in vivo. Use of a three dimensional gel is a prerequisite for the differentiation and fusion of endothelial cells into tubes. HUVECS grown on the surface of gelatin gels or on plastic do not undergo tube-formation.

The method used to quantitate endothelial cell gene expression was Quantitative Expression Analysis (“QEA”, See U.S. Pat. No. 5,871,697; entitled ‘Method and Apparatus for Identifying, Classifying, or Quantitating DNA Sequences in a Sample Without Sequencing’; Shimkets et al., Nature Biotechnology 17:798-803 (1999)).

Using the GeneCalling™ method, over 100 genes that were differentially expressed in HUVECs under the conditions of tube formation were identified. In order to demonstrate that QEA, as practiced, is a valid procedure for identifying genes differentially expressed under conditions of angiogenesis, it was found that four genes previously known to be expressed in HUVECs during angiogenesis were also demonstrated to be differentially expressed in the present study. These genes were:
1-CD31 (PECAM-1) Genbank accession: m28526
2-Thrombospondin Genbank accession: x14787
3-Collagenase-1 (MMP-1) Genbank accession: x05231
4-Interleukin-8 Genbank accession: m28130

The present invention discloses 27 genes, and the proteins encoded by them, that have not been previously identified as being expressed in endothelial cells during differentiation into tube-like structures. For this reason, the genes or gene products represent important therapeutic modalities or therapeutic targets for use in clinical situations associated with angiogenesis. These include certain pathologies suggesting stimulation of angiogenesis on the one hand, and others suggesting suppression of angiogenesis on the other hand.

Included among the genes and gene products that are enhanced are tissue factor pathway inhibitor-2 (PA13), aggrecanase (ADAMTS-4) (PA12), protein KIAA0188, placental growth factor (PIGF) (PA22), fibroblast growth factor 16 (FGF-16) (PA24), stanniocalcin precursor (PA23), tyrosine kinase receptor (epithelial cell ECK) (PA16), interleukin 6 signal transducer (gp130) (PA18), CD82 (PA19), podocalyxin-like protein (PA3), OX40 cell surface antigen (PA17), alpha-2 integrin (PA21), protein zero related protein (PA20), T-plastin (PA7), moesin (PA4), dynein light chain (PA9), myosin-IC (PA6), ankyrin-2 (PA10), actin bundling protein (PA8), osteonidogen (PA1), white protein homolog (PA25), cathepsin B (PA11), and laminin gamma-2 chain (PA2). Included among the genes and gene products that are suppressed in an angiogenic environment are tyrosine kinase receptor ax1 (PA15), urokinase inhibitor (PAI-2) (PA14), and mesothelial keratin K7 (type II) (PA5). Furthermore, additional genes or gene products differentially expressed in angiogenesis that are considered pro-angiogenic for the purposes of the invention include ALG-2 interacting protein (PA26) and collagenase. Also differentially expressed is a novel gene, r0v0-176 (PA27).

A summary of these PA genes that are differentially expressed is included in Table 1.

TABLE 1
Differential Expression Data

| | | GeneCalling | TaqMan |
|---|---|---|---|
| Gene name | Gb acc number | modulation | modulation |
| osteonidogen (PA1) | D86425 | +4 | +2 |
| laminin gamma-2 chain (PA2) | U31201 | +18 | +49 |
| podocalyxin-like protein (PA3) | U97519 | +12 | +2 |
| moesin (PA4) | M69066 | +2 | 1 |
| mesothelial keratin K7 (type II) (PA5) | X03212 | −5 | −10 |
| myosin-IC (PA6) | U14391 | +2 | ND |
| T-plastin (PA7) | L05491 | +2 | 1 |
| actin bundling protein (PA8) | U09873 | +2 | +2 |
| dynein light chain (PA9) | U32944 | +3 | ND |
| C3VS homolog (PA10) | sim to Q28282 | +2 | 1 |
| cathepsin B (P11) | M14221 | +3 | +2 |
| aggrecanase, ADAMTS-4 (PA12) | NM_005099 | +18 | +2 |
| tissue factor pathway inhibitor-2 | L27624 | +9 | +7 |
| (PA13) | | | |
| urokinase inhibitor (PAI-2) (PA14) | M31551 | −19 | −92 |
| tyrosine kinase, receptor ax1, alt. | P30530 | −6 | −15 |
| splice 2 (P15) | | | |
| tyrosine kinase, receptor, epithelial | NM_004431 | +3 | +3 |
| cell, ECK (PA16) | | | |
| OX40 (PA17) | S76792 | +18 | +18 |
| interleukin 6 signal transducer, | M57230 | +3 | 1 |
| gp130 (PA18) | | | |
| cd82 (PA19) | D28137 | +12 | +4 |
| protein zero related protein (PA20) | AF087020 | +6 | +6 |
| alpha-2 integrin (PA21) | X17033 | +13 | +2 |
| placenta growth factor (PIGF) (PA22) | X54936 | +6 | +5 |
| stanniocalcin precursor (PA23) | U25997 | +14 | +8 |
| Fibroblast growth factor (FGF-16) | AB009391 | +4 | 1 |
| (PA24) | | | |
| white protein homolog* (PA25) | X91249 | +3 | −2 |
| sim mouse Alix (ALG-2 interacting | sim to AJ005073 | +5 | +2 |
| protein) (PA26) | | | |

*Any discrepancy of modulation between GeneCalling and TaqMan is likely due to a very steep time-dependent mRNA expression in which small experimental variations can cause the observed modulations in the two methods.

In Table 1, a plus sign denotes up-regulation of gene expression, and a minus sign denotes down-regulation. The time frame of these experiments was 24 hr relative to 4 hr in a gel environment that is conducive to tube-formation. “ND” means that the level of modulation was not determined. A summary of the differentially expressed sequences is presented in Table 1. Column 1 provides the common names of each of the genes along with its PA assignment, Column 2 lists the sequence database reference number, Column 3 provides the results of the GeneCalling expression analysis, and Column 4 provides the results of the TaqMan™ expression analysis.

One of ordinary skill in this art will recognize that the information in Table 1 can be used to identify a particular PA sequence. For example, where the sequence database reference number of a particular nucleic acid sequence is known, this reference number can be used to identify a particular PA sequence. For a given PA sequence, its expression can be measured by using any methods commonly known in the art. Based on all of this, one of ordinary skill in the art will be able to deduce the information necessary for detecting and measuring the expression of each PA nucleic acid sequence, as required by each of the methods described herein.

The genes and the polypeptides they encode are believed to be essential components, singly or severally, in the biologic pathway(s) associated with endothelial cell tube formation or anglogenesis.

Angiogenesis is an important component of a variety of diseases and disorders including tumor growth and metastasis, rheumatoid arthritis, psoriasis, atherosclerosis, diabetic retinopathy, retrolental fibroplasia, neovascular glaucoma, age-related macular degeneration, hemangiomas, immune rejection of transplanted corneal tissue and other tissues, and chronic inflammation. Accordingly, the present invention provides means to detect, monitor, analyze, identify, or treat the occurrence or progression of angiogenesis or vasculogenesis in these and other related conditions, and to identify drugs, e.g., antisense, small molecule, antibody, useful to treat these and other related conditions.

Certain of the PA genes that are differentially expressed under angiogenic conditions have enhanced levels of expression, and other differentially expressed PA genes have suppressed expression levels. Accordingly, in pathologies in which it is desirable to inhibit de novo angiogenesis, such as the treatment of a solid tumor, it would be beneficial to administer a therapeutic agent that is an antagonist to a particular activity manifested by such a PA polypeptide whose expression level is enhanced. In pathologies in which it is beneficial to stimulate angiogenesis, such as in wound healing or cardiac myopathy it would be therapeutically beneficial to administer a pharmaceutical agent that serves as an agonist for a particular PA polypeptide whose expression is enhanced during angiogenesis, and/or that provides an antagonistic effect for those PA polypeptides whose expression is suppressed during angiogenesis.

The inventors also showed that certain of the PA genes differentially expressed in angiogenesis have suppressed levels of expression. Accordingly, in pathologies in which it is desirable to inhibit de novo angiogenesis, such as the treatment of a solid tumor, it would be beneficial to administer a therapeutic agent that is an agonist to a particular activity manifested by a PA polypeptide whose expression is suppressed in order to counteract the suppression. In analogous fashion, in a pathology in which angiogenesis is desired to achieve a therapeutic benefit, it would be beneficial to administer a therapeutic agent that is an antagonist for a PA polypeptide that is more poorly expressed in the angiogenesis, thus reinforcing the suppression of the PA polypeptide.

Below follows additional discussion of the nucleic acid sequences that are differentially expressed in angiogenesis. For ease of understanding, these PA nucleic acid sequences have been grouped according to their common features and/or characteristics.

Extracellular Matrix Associated Proteins

Osteonidogen (Nidogen), acc:D86425 [PA1]

Nidogen is usually associated with the basement membrane and interacts with collagens I and IV, laminin, and perlecan-involved in cell-cell adhesion and basement membrane organization (See Nicosia et al., Dev Biol, 164:197-206, 1994; Kohfeldt et al., J Mol Biol 282, 99-109, 1998).

TaqMan™ analysis was performed as described in Example 19 using the primers and probe given in Table 4. The results for osteonidogen gene expression are shown in FIG. 3. It is seen that osteonidogen expression is profoundly enhanced over the 2 day course of cell growth; expression is somewhat higher in cells grown under conditions of tube formation.

Laminin Gamma-2 Chain, acc:U31201 [PA2]

Laminin gamma-2 chain links proteins of basal lamina. It consists of an A-chain (400 kD) and two B-chains (200 kD). Each subunit contains at least 12 repeats of the EGF-like domain. Laminin induces adhesion and spreading of many cell types and promotes the outgrowth of neurites in culture. The major components of basal laminae are the glycoproteins laminin and collagen IV, both of which are heterotrimers. Laminin is a cruciform protein trimer of chains that, when originally isolated from the extracellular matrix of tumor cells, were named A, B1, and B2, but were renamed alpha-1, beta-1, and gamma-1, respectively. Several isoforms of each chain have been identified. Laminins containing gamma-1 chain have been shown to have been shown to be important for cell differentiation, adhesion, migration, and neurite outgrowth (See Hager et al., Neuroscience 86:1145-54, 1998). Several peptides derived from laminin gamma-1 chain have the property of promoting endothelial cell adhesion and have angiogenic activity (See Ponce et al., Cir Res 84:688-94, 1999). However, laminins containing gamma-2 chain has not previously been shown to be associated with these processes.

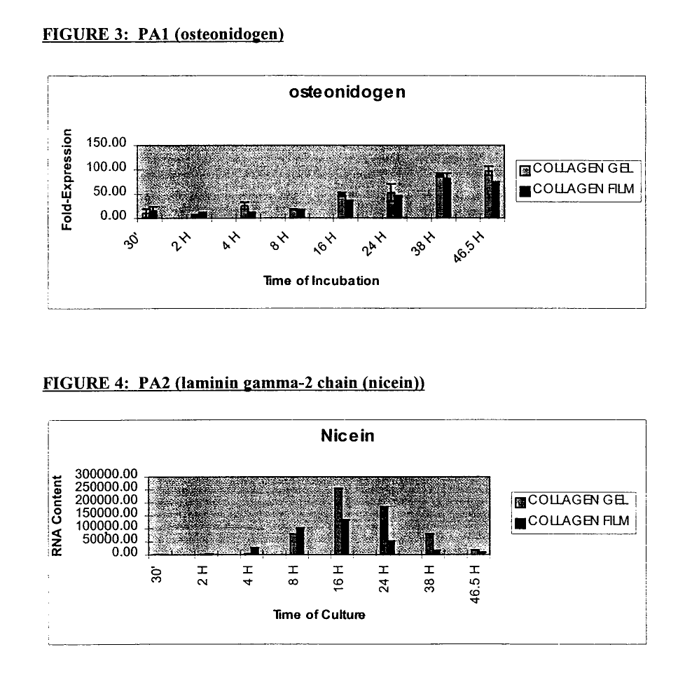

TaqMan™ analysis was performed as described in Example 19 using the primers and probe given in Table 4. The results for laminin are shown in FIG. 4. Expression of laminin gammna-2 chain is profoundly enhanced under tube forming conditions (gel) compared to cells grown under conditions that do not foster tube formation (film). The expression peaks at 16-24 hours, suggesting that laminin expression contributes to early stages of growth.

Podocalyxin-Like Protein, acc: U97519 [PA3]

PA3 is similar to podocalyxin in glomeruler filter, and vascular endothelium. It is a sialoprotein that apparently binds L-selectin in endothelial cells. Podocalyxin-like protein has been suggested to be involved in adhesion and lymphocyte rolling. Podocalyxin is a constituent of the endothelial plasma membrane. The function of podocalyxin in endothelial cells remains enigmatic, although recently it was shown that podocalyxin could function as an L-selectin receptor in inflammed lymph nodes (See Sassetti et al., J Exp Med 187:1965-75, 1998). Thus this receptor might play some role in cell-cell interactions or adhesion.

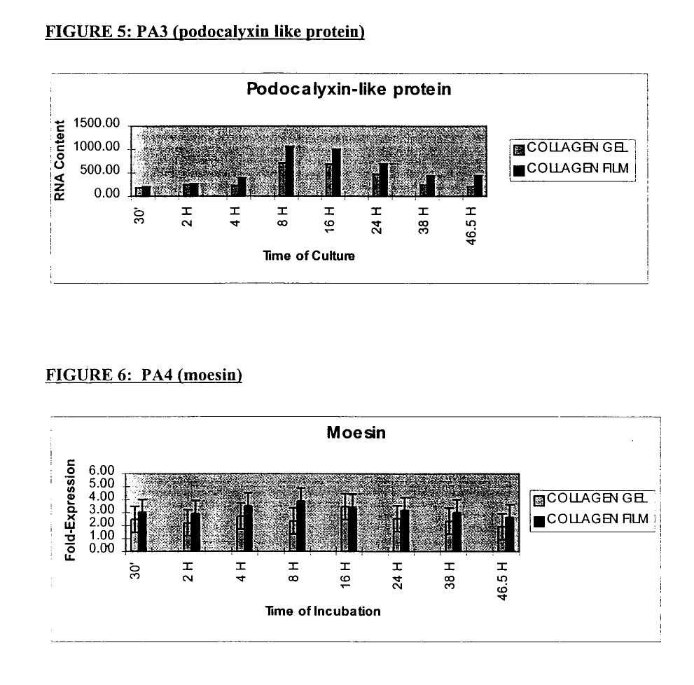

TaqMan™ analysis was performed as described in Example 19 using the primers and probe given in Table 4. The results for podocalyxin like protein are shown in FIG. 5. Expression of the gene is stronger under conditions that do not foster tube formation (film) than cells grown under tube forming conditions (gel). The expression peaks at 8-16 hours, suggesting that laminin expression contributes to early stages of growth.

Cytoskeletal Proteins

Moesin, acc: M69066 [PA4]

Moesin (membrane-organizing extension spike protein) is a candidate receptor for heparin or heparin sulfate in the interaction of basement membrane heparan sulfate and cells. It is has significant sequence homology to ezrin, protein 4.1, talin, radixin, and merlin. These proteins constitute a family with structural, and probably functional, relationships. All of these proteins are localized to the submembranous cytoskeleton. Additionally, moesin is widely expressed in different tissues, where it is localized to filopodia and other membranous protrusions that are important for cell-cell recognition and signaling and for cell movement (See Amieva and Furthmayr Exp Cell Res 219:180-96, 1995).

TaqMan™ analysis was performed as described in Example 19 using the primers and probe given in Table 4. The results for moesin are shown in FIG. 6. Expression of moesin is consistently higher under conditions that do not foster tube formation than under conditions that do. The expression level is high at all times examined.

Keratin K-7, acc: X03212 [PA5]

Cytokeratins (also known as keratins) are intermediate filament proteins that are expressed primarily in epithelial cells (See Alberts et al.). The gene for the simple epithelial keratin K7 has been assigned to chromosome 12 by use of Southern blot analysis of somatic cell hybrids. Specifically, the gene has been localized to region 12q12-q14 by in situ hybridization of metaphase chromosomes. Keratins are often used as markers for various types of tumors (See Hibino et al., J Invest Dermato 112:85-90, 1999; Sundstrom and Stigbrand Int J Biol Markers 9:102-8, 1994).

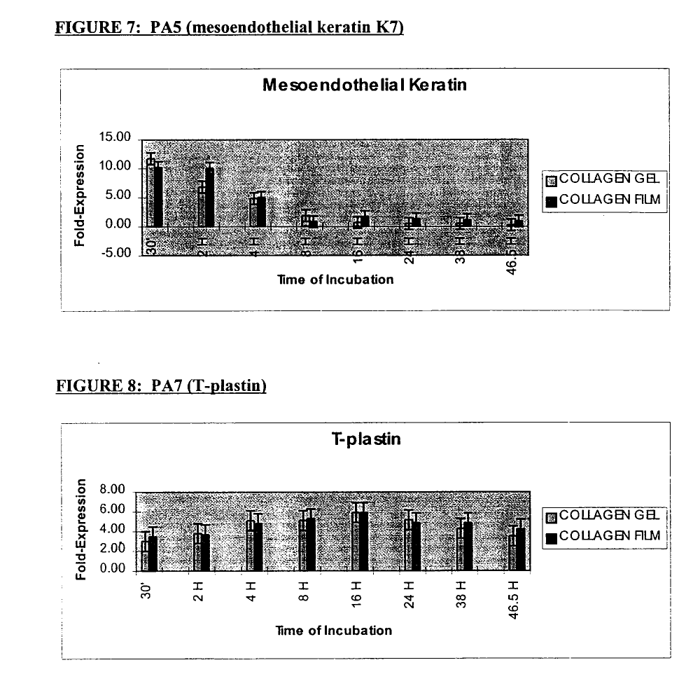

TaqMan™ analysis was performed as described in Example 19 using the primers and probe given in Table 4. The results for mesoendothelial keratin are shown in FIG. 7. It is seen that mesoendothelial keratin expression is strongly suppressed in growing HUVECs, and is more profoundly suppressed under conditions of tube formation (gel) than on film. These results suggest that stimulation of expression of mesoendothelial keratin would be useful in clinical situations in which angiostasis is desired.

Myosin-IC, acc: U14391 [PA6]

The class I myosins are single-headed, actin-binding, mechanochemical “motor” proteins with heavy chains in the molecular mass range of 110-130 kDa. These proteins do not form filaments. Each myosin I heavy chain is associated with one to six light chains that bind to specific motifs known as IQ domains. In vertebrate myosin I isoforms, the light chain is calmodulin, which is thought to regulate motor activity (See Coluccio J Cell Sci 107:2279-84, 1994). Proteins similar to calmodulin are associated with myosin I isoformns from lower eukaryotes. Some myosin I isoforms from lower eukaryotes are regulated by phosphorylation. However, the phosphorylation site is not present in vertebrate myosin I isoforms.

T-Plastin, acc: L05491 [PA7]

Plastins are a family of actin-binding proteins that are conserved throughout eukaryote evolution and expressed in most tissues of higher eukaryotes (See Lin et al., J Biol Chem 268:2781-92, 1993; Arpin et al., J Cell Biol 127:1995-2008, 1994). There are two T-plastin isoforms, and their expression appears to be restricted to replicating cells of solid tissues. However, L-plastin is induced in many human solid tumor-derived cells. Both plastin isoforms contain a potential calcium binding site near the N terminus. T-plastin is likely to be involved in cytoskeletal rearrangements. In view of the observed increased expression of T-plastin mRNA in cis-platin resistant tumor cells (See Hisano et al., FEBS Lett 397:101-7, 1996), and its expression during endothelial cell tube-formation reported herein, this gene is likely a good target for combination therapy with antibody based therapeutics together with cis-platin or other chemotherapeutic alkylating or cross-linking agent.

TaqMan™ analysis was performed as described in Example 19 using the primers and probe given in Table 4. The results for T-plastin are shown in FIG. 8. It is seen that T-plastin expression peaks to a modest extent at 8-24 hours of growth. There is little significant difference of expression between cells grown in gel and cells grown on film.

Actin Bundling Protein, acc: U09873 [PA8]

Actin bundling protein is involved in actin filament dynamics. They are also known as fascins and are found in membrane ruffles, microspikes, and stress fibers (See Yamashito et al., Mol Biol Cell 9:993-1006, 1998). Fascin is homologous to the drosophila “signed” gene product (SLN), and the human gene has been called HSN. The heLa-cell derived 55-kD protein is thought to be involved in the assembly of actin filament bundles present in microspikes, membrane ruffles, and stress fibers.

TaqMan™ analysis was performed as described in Example 19 using the primers and probe given in Table 4. The results for actin bundling protein are shown in FIG. 9. Expression of actin bundling protein undergoes a modest increase after 8 hours growth, with slightly greater expression under conditions that do not promote tube formation. This suggests that stimulating expression of actin bundling protein would tend to minimize angiogenesis, and that an antagonistic agent could promote angiogenesis.

Dynein Light Chain, acc: U32944 [PA9]

Cytoplasmic dynein is a microtubule-based mechanochemical protein that plays an essential role in cell division, vesicle transport, and cytoplasmic membrane organization. As a molecular motor, dynein utilizes an ATP hydrolysis mechanism to bind and release microtubules and to undergo conformational changes that result in a net displacement towards the microtubule's minus end (See Samso et al., J Mol Biol 276:927-37, 1998).

C3VS Homolog Similar to C3VS from Canis familiaris (Dog) acc:Q28282 [PA10]

This gene encodes a novel human protein that is homologous to C3VS from Canis familiaris (dog). Part of this cloned sequence, herein referred to as ss_{—}79, has been previously identified and patented as acc:V33528. The patented sequence is partial (254 bp), and has only a partial reading frame that is contained in and is not fully identical to ss_{—}79 disclosed herein. The sequence ss_{—}79 is 1434 bp long and encodes a 402 aa reading frame, is homologous to a dog gene C3VS and is therefore a novel human ortholog of the dog gene. The nucleotide in the sequence denoted in bold and underlined is a misread, which causes a frameshift in the sequence that leads to a premature stop codon. This determination was noted by comparison to the public sequence GenBank Acc:AF155135 released in March 2000.

In addition to ankyrin domains, the protein contains a protein kinase phosphorylation site, and a Src related SH3 domain. It does not appear to be a secreted protein. C3VS homolog upregulation in tube-forming endothelial cells described herein suggests a direct involvement in the process of endothelial differentiation and angiogenesis.

The protein product is most likely involved in cytoskeletal interaction/reorganization during the tube forming phase of angiogenesis. It contains ankyrin repeat domains, a kinase phosphorylation site, and a Src related SH3 domain. It does not appear to be a secreted protein. It's upregulation in the tube-forming endothelial cells described herein suggests a direct involvement in the process of endothelial differentiation and angiogenesis. The presence of the SH3 domain and the ankyrin repeat sequences in this protein strongly suggests that it interacts with cytoskeletal elements during tube formation and that its upregulation in tube-forming endothelial cells is essential to angiogenesis.

The sequence of ss_{—}79 is presented below:

(SEQ ID NO:1)

GGCCGCGTTTTCCTGGGGAAGCGGCGGGCGGGGTGGAGCAGCCAGCTGGG
TCCGGGGAGCGCCGCCGCCGCCTCGATGGGGTGTTGAAAAGTCTCCTCTA
GAGCTTTGGAAGGCTGAATGCACTAAACATGAAGAGCTTGAAAGCGAAGT
TCAGGAAGAGTGACACCAATGAGTGGAACAAGAATGATGACCGGCTACTG
CAGGCCGTGGAGAATGGAGATGCGGAGAAGGTGGCCTCACTGCTCGGCAA
GAAGGGGACCAGTGCCACCAAACACGACAGTGAGGGCAAGACCGCTTTCC
ATCTTGCTGCTGCAAAAGGACACGTGGAATGCCTCAGGGTCATGATTACA
CATGGTGTGGATGTGACAGCCCAAGATACTACCGGACACAGCGCCTTACA
TCTCGCAGCCAAGAACAGCCACCATGAATGCATCAGGAAGCTGCTTCAGT
CTAAATGCCCAGCCGAAAGTGTNGACAGCTCTGGGAAAACAGCTTTACAT
TATGCAGCGGCTCAGGGCTGCCTTCAAGCTGTGCAGATTCTCTGCGAACA
CAAGAGCCCCATAAACCTCAAAGATTTGGATGGGAATATACCGCTGCTNC
TTGCTGTACAAAATGGTCACAGTGAGATCTGTCACTTTCTCCTGGATCAT
GGAGCAGATGTCAATTCCAGGAACAAAAGTGGAAGAACTGCTCTCATGCT
GGCCTGTGAGATTGGCAGCTCTAACGCTGTGGAAGCCTTAATTAAAAAGG
GTGCAGACCTAAACCTTGTAGATTCTCTTGGATACAATGCCTTACATTAT
TCCAAACTCTCAGAAAATGCAGGAATTCAAAGCCTTCTATTATCAAAAAT
CTCTCAGGATGCTGATTTAAAGACCCCAACAAAACCAAAGCAGCATGACC
AAGTCTCTAAAATAAGCTCAGAAAGAAGTGGAACTCCAAAAAAACGCAAA
GCTCCACCACCTCCTATCAGTCCTACCCAGTTGAGTGATGTCTCTTCCCC
AAGATCAATAACTTCGACTCCACTATCGGGAAAGGAATCGGTATTTTTTG
CTGAACCACCCTTCAAGGCTGAGATCAGTTCTATACGAGAAAACAAAGAC
AGACTAAGTGACAGTACTACAGGTGCTGATAGCTTATTGGATATAAGTTC
TGAAGCTGACCAACAAGATCTTCTCTCTCTATTGCAAGCAAAAGTTGCTT
CCCTTACCTTACACAATAAGGAGTTACAAGATAAATTACAGGCCAAATCA
CCCAAGGAGGCGGAAGCAGACCTAAGCTTTGACTCATACCATTCCACCCA
AACTGACTTGGGCCCATCCCTGGGGAAAACCTGGTGAAACCTCTCCCCCA
GACTCCAAATCATCTCCATCTGTCTTAATACATTCTTTAGGTAAATCCAC
TACTGGCAATGATGTCAGAATTCAGNCAACTGGC

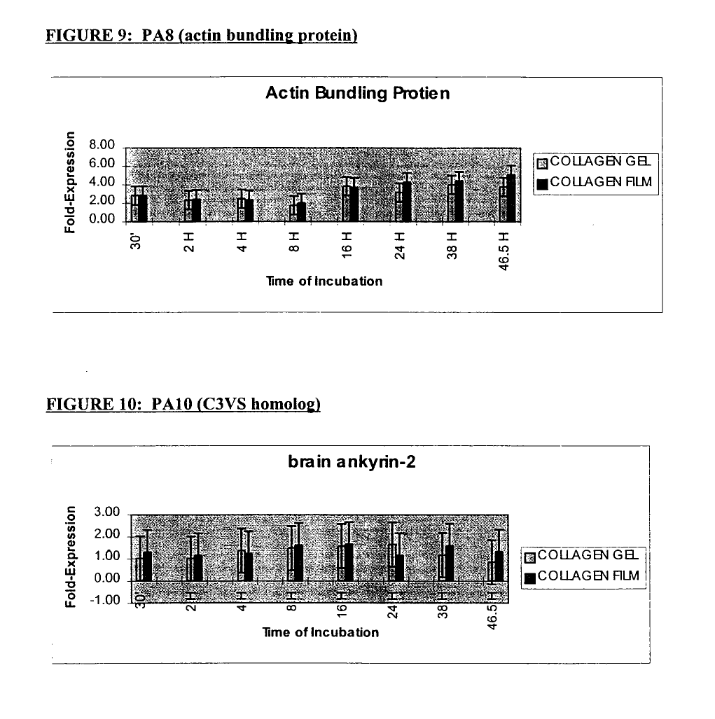

TaqMan™ analysis was performed as described in Example 19 using the primers and probe given in Table 4. The results for brain ankyrin-2 are shown in FIG. 10.

Genes Involved in Protein Degradation, Proteinases/Proteinase Inhibitors

These genes fit into the biologic pathway of matrix degradation during the first phase of angiogenesis.

Cathepsin B, acc:L16510 [PA11]

Cathepsin B is a lysosomal thiol endopeptidase that is involved in extracellular matrix degradation (See Keppler et al., Biochem Cell Biol 74:799-810, 1996). It is likely involved in invasion of endothelial cells into surrounding matrix, and it is involved in antigen degradation, overexpressed in tumors of the lung, prostate, colon, breast, and stomach. Cathepsin B has been linked to tumor progression through observations that its activity, secretion or membrane association are increased. The most malignant tumors, and specifically the cells at the invasive edge of those tumors, express the highest activity. Cathepsin B may facilitate tumor invasion directly by dissolving extracellular matrix barriers like the basement membrane, or indirectly by activating other proteases capable of digesting the extracellular matrix (See Berquin and Sloane Adv Exp Med Biol 389: 281-94, 1996).

TaqMan™ analysis was performed as described in Example 19 using the primers and probe given in Table 4. The results for cathepsin B are shown in FIG. 12. Expression is not significantly different for gel vs film cells; the level of expression peaks modestly at 8-24 hr.

ADAMTS-4 (Aggrecanase), acc:NM_{—}005099 [PA12]

Aggrecanase is a proteolytic enzyme belonging to the family of Disintegrin type Metalloprotease family. This enzyme has been implicated in the turnover of extracellular matrix in pathologic conditions such as osteoarthritis where it degrades aggrecan, the high molecular weight aggregating proteoglycan (See Tortorella et al., Science 284:1664-6, 1999; Vasquez et al., J Biol Chem 274:23349-57, 1999).

TaqMan™ analysis was performed as described in Example 19 using the primers and probe given in Table 4. The results for ADAMTS-4 are shown in FIG. 14. Expression of ADAMTS-4 is profoundly enhanced after 2 hours of growth. Between 30 min and 8 hours, the gene is expressed far more strongly in non-tube forming conditions than in tube forming conditions. After 8 hours, the expression under both conditions is comparable. These results suggest that growth of endothelial cells requires early expression of this gene, and that, based on the early time results, tube formation, a model for vascularization, would be inhibited if its expression were enhanced.

Human BAC Clone GS345D13 from 7q31-q32, Complete Sequence, acc:L27624 [PA13]

This is also known as tissue factor pathway inhibitor 2, a Kunitz type serine protease inhibitor. Human BAC clone GS345D13 obtained from 7q31-q32. Its complete sequence is found at accession number AC002076. PA13 is a protease inhibitor, also known as placental protein 5, is a serine protease inhibitor consisting of three tandemly-arranged Kunitz-type protease inhibitor domains. TFPI-2 is a potent inhibitor of trypsin, plasmin, kallikrein, and factor Xia; likely involved in the regulation of degradation of extracellular matrix during the formation of tubes, and adhesion of endothelial cells to ECM (See lino et al., Arteriosclerosis, Thrombosis, and Vascular Biology, 18:40-6, Rao et al., Biochem Biophys Res Comm 255:94-8, 1999). Tissue factor pathway inhibitor is an important regulator of the extrinsic pathway of blood coagulation through its ability to inhibit factor Xa and factor VIIa-tissue factor activity. It was originally identified as a placental glycoprotein that inhibits plasmin, trypsin, and thrombin and has been referred to as placental protein-5. PA13 has been reported to exhibit mitogenic activity in smooth muscle cells (See Shinoda et al., J Biol Chem 274:5379-84, 1999).

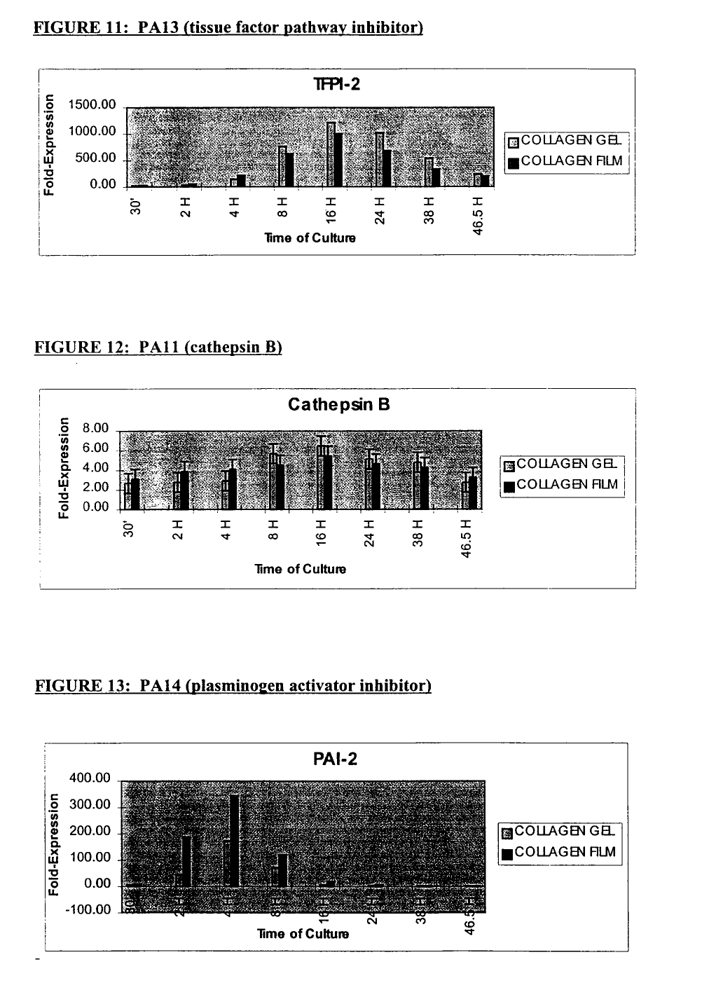

TaqMan™ analysis was performed as described in Example 19 using the primers and probe given in Table 4. The results for tissue factor pathway inhibitor are shown in FIG. 11. It is seen that expression of this gene is profoundly enhanced at 16-24 hr, with slightly greater expression under conditions of tube formation than on film. This suggests that stimulation of tissue factor pathway inhibitor may promote angiogenesis, and that an antagonist of the gene or protein may foster angiostasis.

Urokinase Inhibitor (PAI-2) acc:M31551 [PA14]

PA14 is a member of the serpin family of proteins. It inhibits both the tissue-type and urokinase-type plasminogen activators. Previous studies have shown expression of this gene by a variety of cell types including endothelial cells (See Amman et al., Thrombosis Res 77:431-40, 1995; Zoellner et al., Thrombosis and Haemostasis 69:135-40, 1993). However, the modulation of this gene during endothelial tube formation has not been described previously. Down-regulation of PAI-2 in endothelial cells likely increases the degradation of extracellular matrix and is conducive to invasion of endothelial cells into surrounding matrix. PAI-2 has been suggested to play a role in apoptosis induced by TNF-α (See Dickinson et al., Cell Death Differ 5:163-71, 1998).

TaqMan™ analysis was performed as described in Example 19 using the primers and probe given in Table 4. The results for plasminogen activator inhibitor-2 are shown in FIG. 13. Expression of tissue plasminogen activator-2 mRNA peaks dramatically at 4 hours of growth, and is profoundly stronger in cells grown on film (no tube formation) than in cells grown in gel (promoting tube formation). These results suggest that growth of endothelial cells requires early expression of this gene, and that tube formation, a model for vascularization, would be inhibited if its expression were enhanced.

Tyrosine Kinase Receptors

In endothelial cells, these take part in the signaling pathways that lead to tube formation.

Ax1, acc: X57019 [PA15]

The ax1 receptor has a structure that is unique among tyrosine kinases. It was first identified as an mRNA overexpressed during the progression of chronic myelogenous leukemia (CML) to acute-phase leukemia; it drives cell proliferation when the receptor is stimulated. Ax1 is a receptor tyrosine kinase that contains both immunoglobulin and fibronectin III repeats in its extracellular domain reminiscent of cell adhesion molecules (See McCloskey et al., J Biol Chem 272:23285-91, 1997), and has been shown to be involved in granulocyte adhesion to endothelium (Avanzi et al., Blood 91:2334-40. 1998). The growth arrest-specific gene-6 (GAS-6) has been identified as an ax1 stimulatory factor. Exogenous Gas6 protected HUVECs from apoptosis in response to growth factor withdrawal and from TNFalpha-mediated cytotoxicity (See O'Donnell et al., Am J Pathol 154:1171-80, 1999). Therefore, ax1 is likely involved in the survival mechanism of endothelial cells. See also U.S. Pat. No. 5,468,634.

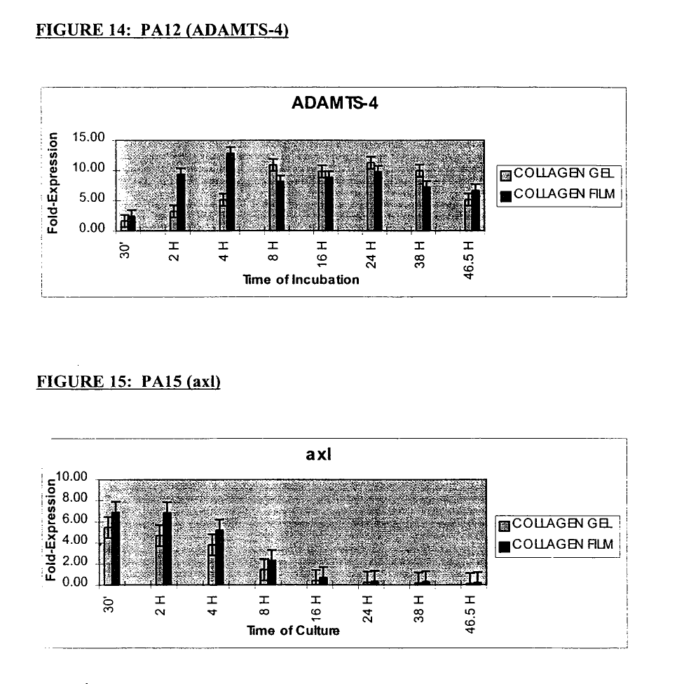

TaqMan™ analysis was performed as described in Example 19 using the primers and probe given in Table 4. The results for ax1 are shown in FIG. 15. Expression of ax1 is strongly suppressed after 8 hours of growth, with the extent of suppression comparable in tube-forming and non-tube-forming conditions. Since the gene is suppressed under conditions of endothelial cell growth, it possible that stimulation of ax1 gene expression would promote angiostasis.

ECK-1 acc:M59371, NM_{—}004431.1 [PA16]

Epithelial cell receptor, tyrosine kinases (EPH and EPH-related proteins) comprise the largest subfamily of receptor protein-tyrosine kinases. They have been implicated in mediating developmental events, particularly in the nervous system (See Zhou, Pharmacol Ther 77:151-81, 1998). Receptors in the Eph subfamily typically have a single kinase domain and an extracellular region containing a Cys-rich domain and 2 fibronectin type III repeats. The ligands for Eph receptors have been named ephrins. Mice lacking ephrinB2 and a proportion of double mutants deficient in EphB2 and EphB3 receptor signaling die in utero before embryonic day 11.5 because of defects in the remodeling of the embryonic vascular system (See Adams et al., Genes Dev 13:295-306, 1999). The modulation of this gene in endothelial tube-formation process has not been previously described, therefore the precise role of Eck-1 in angiogenesis is defined herein as its involvement in tube formation.

TaqMan™ analysis was performed as described in Example 19 using the primers and probe given in Table 4. The results for ECK are shown in FIG. 16. Gene expression peaks modestly at 8-24 hours, with little significant difference between cells grown in gel and cells grown in film.

Transmembrane Receptors

OX-40 acc:S76792 [PA17]

OX-40 is a type 1 transmembrane glycoprotein previously found to be expressed in activated T-cells and is thought to play a role in T-cell adhesion to HUVECs (See Imura et al., J Exp Med 183:185-95, 1996). It is a member of TNF-alpha receptor family (See Arch and Thompson Mol Cell Bio 18:558-65, 1998). The OX40 ligand is a type II membrane bound cytokine identified as glycoprotein 34 (gp34), a member of the TNF family (See Godfrey et al., J Exp Med 180:757-62, 1994).

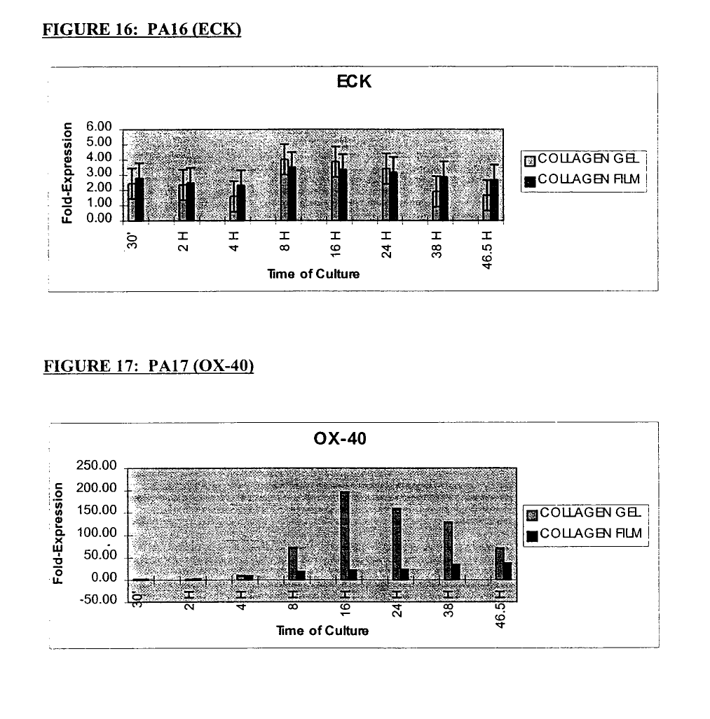

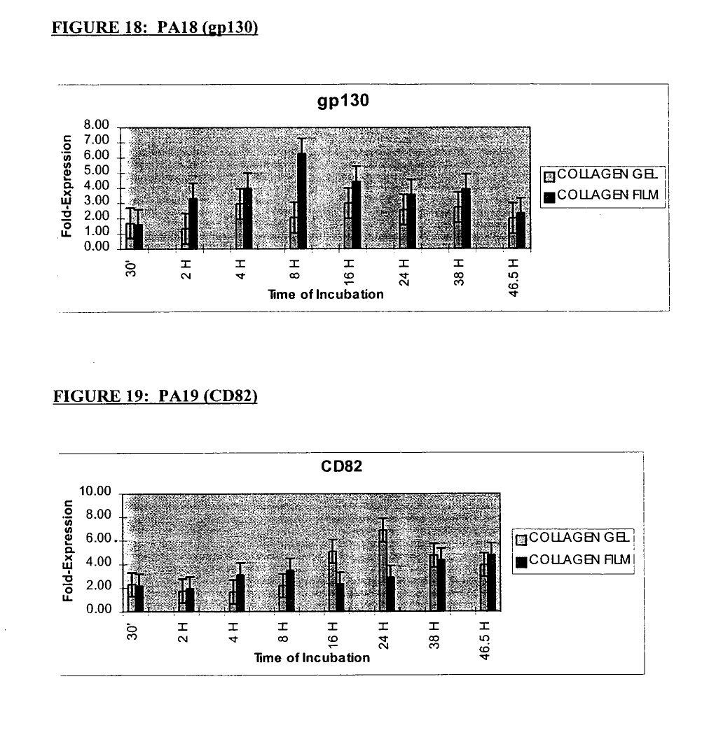

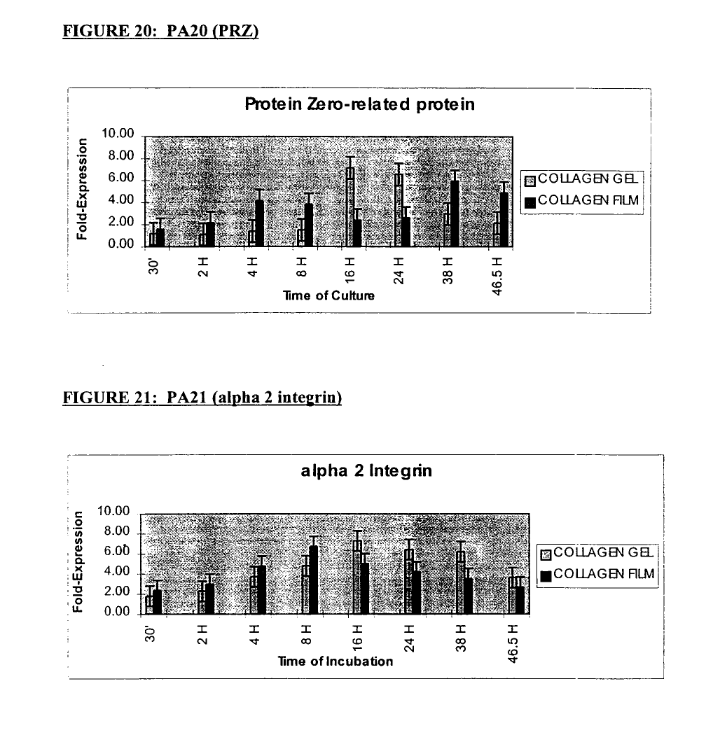

TaqMan™ analysis was performed as described in Example 19 using the primers and probe given in Table 4. The results for OX-40 are shown in FIG. 17. Expression of this gene under tube-forming conditions is dramatically enhanced after 8 hours growth, whereas under conditions that do not foster tube formation its expression is only minimally affected. These results strongly suggest treatment with, for example, an agonist of OX-40 under clinical situations in which angiogenesis is desired would be beneficial.

Interleukin 6 Signal Transducer, gp130 acc:M57230 [PA18]

Initially described as the interleukin-6 signal transducer, gp130 is a transducer chain shared by many cytokines, such as IL-6, IL-11, leukemia inhibitory factor, oncostatin M, and ciliary neurotrophic factor. All of these cytokines act via a bi- or tripartite receptor complex in which signaling is triggered by homodimerization (IL-6) or heterodimerization with LIF-Rb/gp190 protein (IL-11, LIF, OSM, and CNTF) of gp130 (See Taga and Kishimoto Annual Rev Immunology 15: 797-819, 1997). The extracellular part of the signal transducer gp130 consists of six fibronectin type III-like domains. It has recently been shown that the three membrane distal domains bind to the IL-6-IL-6R complex. A structural model of the IL-6.IL-6R.gp130 complex enabled us to propose amino acid residues in these domains of gp130 interacting with IL-6 bound to its receptor.

TaqMan™ analysis was performed as described in Example 19 using the primers and probe given in Table 4. The results for gp130 are shown in FIG. 18. Expression of gp130 is considerably enhanced under non-tube-forming conditions after 4 hours of growth. It is less strongly expressed in cells grown in gel. These results suggest that growth of endothelial cells requires expression of this gene, and that tube formation, a model for vascularization, would be inhibited if its expression were enhanced.

cd82/BST-2 acc:D28137 [PA19]

CD82 (BST-2, C33) is a member of transmembrane 4 super family that is associated with the MHC class II compartment (See Hammond et al., J Immunology 161:3282-91, 1998). It is an activation Ag of T-cells. Recent studies have shown that CD82 associates with CD4 or CD8 and delivers costimulatory signals for the TCR/CD3 pathway (See Nagira et al., Cell Immoral 157:144-57, 1994). The expression of cd82 is inversely associated with tumor progression and is a favorable prognostic factor in some tumors, an activation antigen of T-cells.

TaqMan™ analysis was performed as described in Example 19 using the primers and probe given in Table 4. The results for CD82 are shown in FIG. 19. Expression of this gene under tube-forming conditions is strongly enhanced between 8 and 24 hours of growth, whereas under conditions that do not foster tube formation its expression is much lower at these times. These results suggest treatment with, for example, an agonist of CD82 under clinical situations in which angiogenesis is desired would be beneficial.

Zero-Related Protein acc: AF087020 [PA20]

PA20 is a protein with a single transmembrane segment and a signal sequence. The extracellular portion of the protein contains a single immunoglobulin-like domain displaying 46% sequence identity to that of myelin P0, a major structural protein of peripheral myelin. The intracellular segment of the protein shows no significant sequence identity to any known protein except for two immunoreceptor tyrosine-based inhibitory motifs, named PZR for protein zero related, suggested to be a putative substrate of tyrosine phosphatase SHP2, may have an important in cell signaling (See Seue Zhao and Zhao, J. Biol. Chem. 273:29367(1998)).

TaqMan™ analysis was performed as described in Example 19 using the primers and probe given in Table 4. The results for protein zero related protein are shown in FIG. 20. Expression of this gene is greater in gel than on film between 16 and 24 hours, but stronger on film than in gel before 8 hours and after 16 hours.

Integrin Alpha-2 Subunit, acc: X17033 [PA21]

The integrin family includes cell surface receptors for extracellular matrix components as well as receptors involved in various aspects of leukocyte adhesion. The integrins generally consist of alpha-beta heterodimeric transmembrane glycoproteins in which the alpha subunit is noncovalently associated with the beta subunit. Originally identified on T cell lymphocytes, they have been subsequently shown to be present on a wide variety of cell types including fibroblasts and platelets. Six forms, VLA-1 to VLA-6, have been identified, each consisting of a distinct alpha chain (numbered alpha-1 to alpha-6) associated with a common beta chain. The alpha-2 heterodimer may function as a cell surface receptor for collagen.

TaqMan™ analysis was performed as described in Example 19 using the primers and probe given in Table 4. The results for alpha 2 integrin are shown in FIG. 21. Expression of the alpha 2 integrin gene peaks at about 16 hours, and is stronger in gel than on film at 16-38 hours. These results suggest that promoting the expression of alpha 2 integrin in situations in which angiogenesis is desired might be beneficial.

Secreted Proteins, e.g. Growth Factors, Cytokines, Peptide Hormones

PIGF, acc: X54936 [PA22]

PIGF Placenta growth Factor is related to VEGF and VPF, a secreted growth factor with angiogenesis regulatory properties. It was previously found to be associated with angiogenesis, a finding, which was recapitulated in this study. It supports the process of in vitro tube formation as a model system for angiogenesis. PA22 also supports the statement that these DNAs and related protein sequences are, in fact, expressed during angiogenesis, because this molecule is a known to be expressed during in vitro and in vivo angiogenesis. PIGF is a member of the vascular endothelial growth factor (VEGF) family of growth factors. PIGF displays a 53% identity with the platelet-derived growth factor-like region of VEGF. By alternative splicing of RNA, two PIGF isoforms are generated namely PIGF131 (PIGF-1) and PIGF152 (PIGF-2). Relative to PIGF131, PIGF152 has a 21-amino acid insertion enriched in basic amino acids. Like VEGF, the PIGF isoforms are homodimeric glycoproteins. PIGF131 is a non-heparin binding protein, whereas PIGF152 strongly binds to heparin. While the PIGF proteins bind with high affinity to Flt-1, they fail to bind to Flk-1/KDR. Purified PIGF isoforms had little or no direct mitogenic or permeability-enhancing activity. However, they are able to significantly potentiate the action of low concentrations of VEGF in vitro and in vivo (See Khaliq, et al., Lab Invest 78:109-116, 1998; Kaliq et al., Growth Factors 13:243-250, 1996; Park et al., J Biol Chem 269: 25646-25654, 1994; Sawano et al., Cell Growth Differ 7:213-221, 1996; Ziche et al, Lab Invest 76:517-531, 1997).

TaqMan™ analysis was performed as described in Example 19 using the primers and probe given in Table 4. The results for placental growth factor are shown in FIG. 22. Expression of this gene under tube-forming conditions is dramatically enhanced after 4 hours growth, whereas under conditions that do not foster tube formation its expression increases more modestly and more slowly. These results strongly suggest treatment with, for example, an agonist of placental growth factor or an agent that promotes increased expression under clinical situations in which angiogenesis is desired would be beneficial.

Stanniocalcin Precursor, acc: U25997 [PA23]

This is a secreted glycoprotein that has been suggested to be involved in calcium and phosphate regulation (See Olsen et al, Proc Natl Acad Sci 93:1792-1796, 1996). Previous studies have shown it is expressed in the kidney and in thymic stromal cells (See Wagner et al, Proc Natl Acad Sci 92:1871-1875, 1995). It is upregulated in endothelial cells differentiating into tube-like structures. This suggests that stanniocalcin may be involved in endothelial tube formation. Since it is a secreted hormone, this suggests the existence of a receptor for stanniocalcin that may be used as a target to block tube formation. Neutralizing antibodies to stanniocalcin may be useful as therapeutic molecules because they bind to stanniocalcin and thereby remove it from the immediate cellular environment.

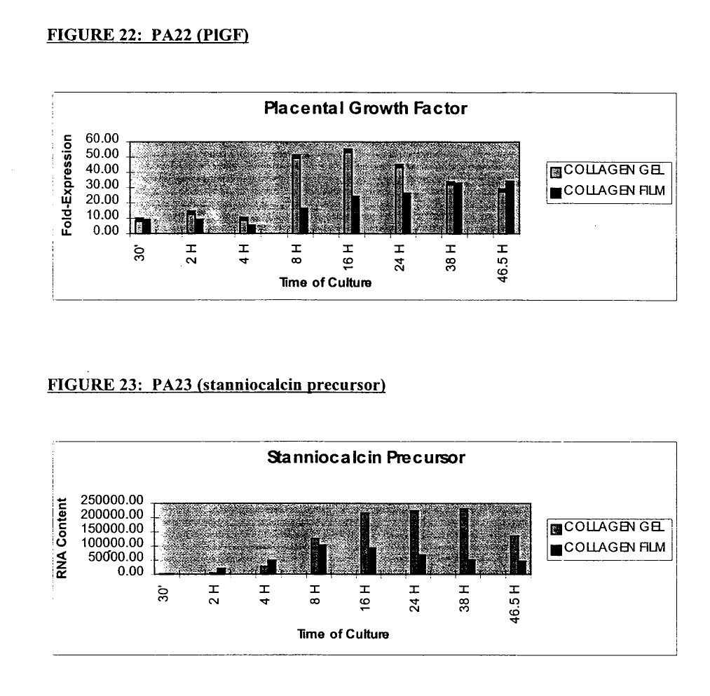

TaqMan™ analysis was performed as described in Example 19 using the primers and probe given in Table 4. The results for stanniocalcin precursor are shown in FIG. 23. Expression of this gene under tube-forming conditions is dramatically enhanced after 8 hours growth, whereas under conditions that do not foster tube formation its expression remains more modest. These results strongly suggest treatment with, for example, an agonist of stanniocalcin precursor or an agent that promotes increased expression under clinical situations in which angiogenesis is desired would be beneficial.

Fibroblast Growth Factor-16, acc:AB009391 [PA24]

Fibroblast growth factors are peptide-regulatory factors acting through 4 distinct tyrosine kinase receptors and involved in various biologic processes during embryogenesis and adult life, including implantation, morphogenesis, angiogenesis, and possibly tumorigenesis. FGF-16 in embryos might play a role in development of the brown adipose tissue. Among FGF family members, FGF-16 is most similar (73% amino acid identity) to FGF-9. Human FGF-16 has a high degree of amino acid sequence identity (98.6%) to rat FGF-16. Up-regulation of FGF-16 is in endothelial cells in tube forming environment suggests that it is involved in the process of angiogenesis (See Miyake et al., Biochem Biophys Res Comm 243:148-152, 1998; Danilenko et al., Arch Biochem Biophys 361:34-46, 1999).

FGF-16 is the most recent member of the FGF family to be cloned (Miyake, 1998). In the rat, FGF-16 was most highly expressed in heart. The human homolog of FGF-16 was also recently cloned from human heart cDNA (Miyake, 1998). The only known activities of FGF-16 are the relatively weak stimulation of NIH 3T3 fibroblast proliferation, and reasonably potent stimulation of primary rat olgodendrocyte proliferation. (Danilenko, 1999).

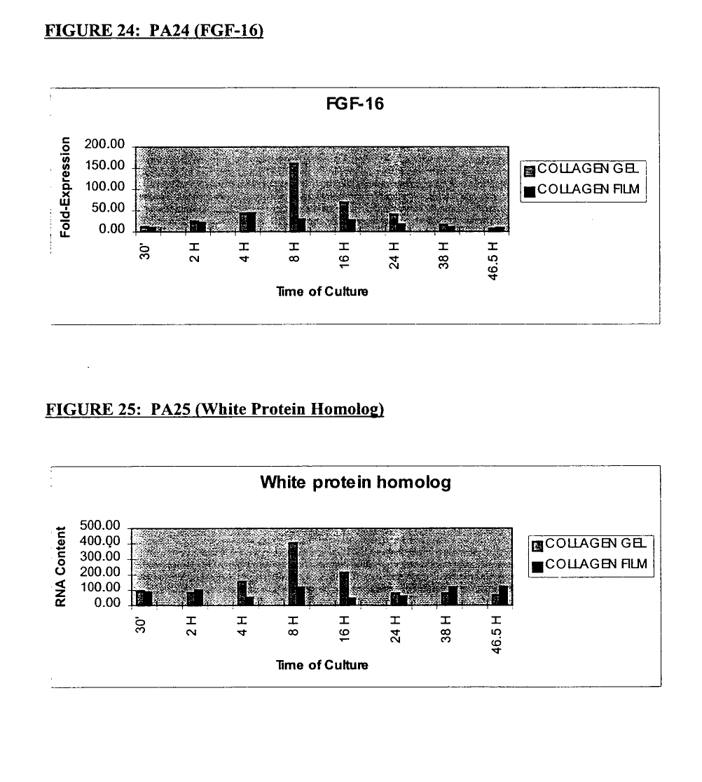

TaqMan™ analysis was performed as described in Example 19 using the primers and probe given in Table 4. The results for FGF-16 are shown in FIG. 24. Expression of this gene under tube-forming conditions is peaks sharply at 8 hours growth, whereas under conditions that do not foster tube formation its expression remains modest. The expression levels then recede to very low levels by 2 days.

To date, there are no publications concerning the possible role of FGF-16 in angiogenesis or expression of this growth factor at sites of new blood vessel formation. Since proliferation is not observed in the HUVEC model system used here for expression analysis, the late time down-regulation observed for FGF-16 is consistent with published reports. However the transient profound enhancement of expression in the absence of proliferation, i.e., associated essentially exclusively with tube morphogenesis, that is observed as shown in FIG. 24, is unexpected. These results strongly suggest treatment with, for example, an agonist of FGF-16 or an agent that promotes increased FGF-16 expression under clinical situations in which angiogenesis is desired would be beneficial.

Channels/Transporters

White Protein Homolog, acc: X91249 [PA25]

A contig of ˜2200 bp comprising 25 component ESTs was made. By BLAST-X it had 99% amino acid homology to human G02068 white homolog. The genomic clone similar to white protein homolog. It is involved in metabolism, metabolite storage/transport proteins, plasma membrane shuttling.

Human White gene protein (HSWHITE) is involved in metabolism, metabolite storage/transport proteins, and plasma membrane shuttling. It is a homolog of the drosophila White gene. In Drosophila, the ‘White’ (W), ‘Scarlet’ (St), and ‘Brown’ (Bw) proteins are members of the ATP-binding cassette (ABC) transporter superfamily of transmembrane permeases and are involved in transporting precursors of eye pigments. The human white protein homolog is also known as an ATP-binding cassette (ABC). Many members of the ABC family of proteins function as transporters or channels (See Dreesen et al., Mol Cell Biol. 8:5206-15 (1988)). The human homologue gene (hW) has been mapped to chromosome 21q22.3.

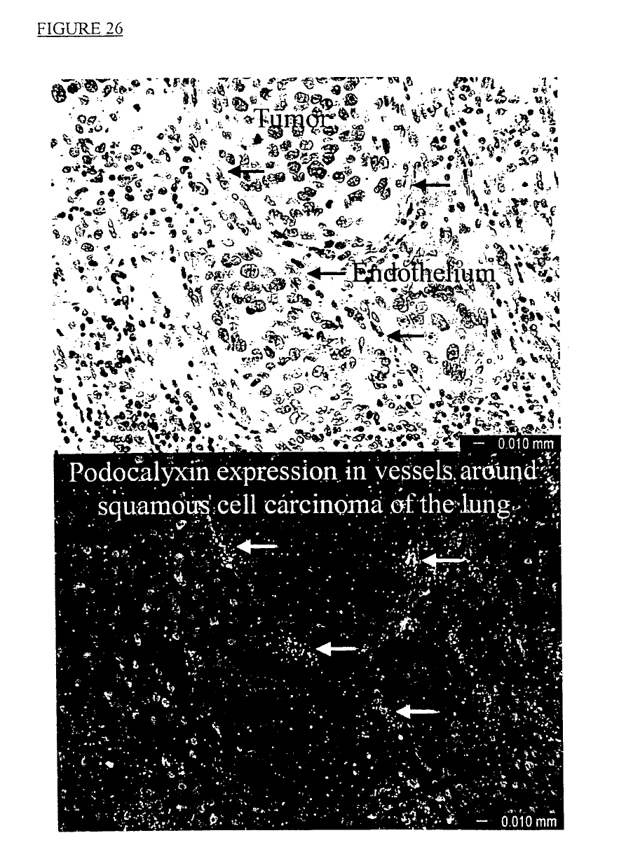

TaqMan™ analysis was performed as described in Example 19 using the primers and probe given in Table 4. The results for white protein homolog are shown in FIG. 25. Expression of this gene under tube-forming conditions is strongly enhanced between 4 and 16 hours growth, whereas under conditions that do not foster tube formation its expression is only minimally affected at these times. These results strongly suggest treatment with, for example, an agonist of white protein homolog under clinical situations in which angiogenesis is desired would be beneficial.

Apoptosis-Related

Gene sim to mouse Alix (AJ005073) or ALG-2 interacting protein-1 (AF151793) [PA26]

GeneCalling identified a cDNA fragment, this EST sequence was used to BLASTN against Genbank composite database, all DNA sequences matching the input DNA by at least 95% over a stretch of 50 bases were used to assemble a contig of 1458 bp that consisted of 15 sequences. These were: est:cb_AA088472+, est:gb_AA196914+, est:gb_AA301555+, est:gb_AA332019+, est:gb_AA337670+, est:gb_AA363712+, est:gb_AA370299+, est:gb_AA984515−, est:gb_AW076072−, est:gb_N56435+, est:gb_N73108+, est:gb_T53906+, est:gb_W38656+, est:gb_W77963+, pg_hs_aj005073_gli0286.3. BLASTN hits human ALG-2 interacting protein, with an exact match over the entire 1458 bp sequence. BLAST-X hit a mouse protein Alix (AJ005073) or ALG-2 interacting protein-1 (AF151793) (p=1.8 e-⁹⁴, matching frame −2, aa329 to 533, 91% identity, 95% positives including conservative substitutions). ALG-2 codes for a Ca(2+)-binding protein required for T cell receptor-, Fas-, and glucocorticoid-induced cell death. It interacts with the apotosis related gene ALG-2. ALG-2 is a newly discovered Ca2+-binding protein that has been demonstrated to be directly linked to apoptosis (See Missotten et al. Cell Death Differ. 6:124-29 (1999)).

The nucleic acid sequence of the consensus extension of aj005073_gli0286.3 using the 15 sequences: est:gb_AA088472+, est:gb_AA196914+, est:gb_AA301555+, est:gb_AA332019+, est:gb_AA337670+, est:gb_AA363712+, est:gb_AA370299+, est:gb_AA984515−, est:gb_AW076072−, est:gb_N56435+, est:gb_N73108+, est:gb_T53906+, est:gb_W38656+, est:gb_W77963+, pg_hs_aj005073_gli0286.3− is presented below.

Human gene similar to mouse Alix (ALG-2 interacting protein):

(SEQ ID NO: 2)

TCTAGATCCTATTGGCAAAGCCACACTTGTGAAATCTACCCCGGTCAATG
TACCCATCAGTCAGAAATTTANTGATCTGTTTGAGAAGATGGTTCCCGTG
TCAGTACAGCAGTCTTTGGCTGCCTATAATCAGAGGAAAGCCGATTTGGT
TAACAGATCAATTGCTCAGATGAGAGAAGCCACCACTTTGGCAAATGGGG
TGCTAGCTTCCCTTAATCTTCCAGCAGCAATTGAAGATGTGTCTGGAGAC
ACTGTACCTCAGTCTATATTGACTAAATCCAGATCTGTGATTGAACAGGG
AGGCATCCAGACTGTTGATCAGTTGATTAAAGAACTGCCTGAATTACTGC
AACGAAATAGAGAAATCCTAGATGAGTCATTAAGGTTGTTGGATGAAGAA
GAAGCAACCGATAATGATTTAAGAGCAAAATTTAAGGAACGTTGGCAAAG
GACACCATCCAATGAACTGTATAAGCCTTTAAGAGCAGAGGGAACCAACT
TCAGAACAGTTTTAGATAAAGCTGTGCAGGCAGATGGACAAGTGAAAGAA
TGTTACCAGTCTCATCGTGACACCATCGTGCTTTTGTGTAAGCCAGAGCC
TGAGCTGAATGCTGCCATCCCTTCTGCTAATCCAGCAAAGACCATGCAGG
GCAGTGAGGTTGTAAATGTCTTAAAATCCTTATTGTCAAATCTTGATGAA
GTAAAGAAGGAAAGAGAGGGTCTGGAGAATGACTTGAAATCTGTGAATTT
TGACATGACAAGCAAGTTTTTGACAGCCCTGGCTCAAGATGGTGTGATAA
ATGAAGAAGCTCTTTCTGTTACTGAACTAGATCGAGTCTATGGAGGTCTT
ACAACTAAAGTCCAAGAATCTCTAAAGAAACAGGAGGGACTTCTTAAAAA
TATTCAGGTCTCACATCAGGAATTTTCGAAAATGAAACAATCTAATAATG
AAGCTAACTTAAGAGAAGAAGTTTTGAAGAATTTAGCTACTGCATATGAC
AACTTTGTTGAACTTGTAGCTAATTTGAAGGAAGGCACAAAGTTTTACAA
TGAGTTGACTGAAATCCTGGTCAGGTTCCAGAACAAATGCAGTGATATAG
TTTTTGCACGGAAGACAGAAAGAGATGAACTCTTAAAGGACTTGCAACAA
AGCATTGCCAGAGAACCTAGTGCTCCTTCAATTCCTACACCTGCGTATCA
GTCCTCACCAGCAGGAGGACATGCACCAACTCCTCCAACTCCAGCGCCAA
GAACCATGCCGCCTACTAAGCCCCAGCCCCCAGCCAGGCCTCCACCACCT
GTGCTTCCAGCAAATCGAGCTCCTTCTGCTACTGCTCCATCTCCAGTGGG
GGCTGGGACTGCTGCGCCAGTTCCATCAACAAACGCCTGGCTCAGCTCCT
CCTCCACAGGCGCAGGGACCACCCTATCCCACCTATCCAGGATATCCTGG
GTATTGCC

Table 2 includes a summary of the PA genes and their major new use categories.

TABLE 2

| Gene name | drug target | antibody target | protein therapeutic | gene therapy | iseaseMarke |
|---|---|---|---|---|---|
| osteonidogen (PA1) | XX | XX | | XX | XX |
| laminin gamma-2 chain (PA2) | xx | xx | | xx | xx |
| podocalyxin-like protein (PA3) | XX | XX | | XX | XX |
| moesin (PA4) | XX | XX | | XX | XX |
| mesothelial keratin K7 (type II) (PA5) | XX | XX | | XX | XX |
| myosin-IC (PA6) | XX | XX | | XX | XX |
| T-plastin (PA7) | XX | XX | | XX | XX |
| actin bundling protein (PA8) | XX | XX | | XX | XX |
| dynein light chain (PA9) | XX | XX | | XX | XX |
| C3VS homolog (PA10) | XX | XX | | XX | XX |
| cathepsin B (PA11) | XX | XX | | XX | XX |
| aggrecanase, ADAMTS-4 (PA12) | XX | XX | | XX | XX |
| tissue factor pathway inhibitor-2 | XX | XX | | XX | XX |
| (PA13) | | | | | |
| urokinase inhibitor (PAI-2) (PA14) | XX | XX | XX | XX | XX |
| tyrosine kinase, receptor ax1, alt. | XX | XX | XX* | XX | XX |
| splice 2 (PA15) | | | | | |
| tyrosine kinase, receptor, epithelial | XX | XX | XX* | XX | XX |
| cell, ECK (PA16) | | | | | |
| OX-40 (PA17) | XX | XX | XX* | XX | XX |
| interleukin 6 signal transducer, | XX | XX | XX* | XX | XX |
| gp130 (PA18) | | | | | |
| cd82 (PA19) | XX | XX | XX* | XX | XX |
| protein zero related protein (PA20) | XX | XX | XX* | XX | XX |
| alpha-2 integrin (PA21) | XX | XX | XX* | XX | XX |
| placenta growth factor (PIGF) (PA22) | XX | XX | XX | XX | XX |
| stanniocalcin precursor (P23) | XX | XX | XX | XX | XX |
| Fibroblast growth factor (FGF-16) | XX | XX | XX | XX | XX |
| (PA24) | | | | | |
| White protein homolog (PA25) | XX | XX | | XX | XX |
| sim mouse Alix (ALG-2 interacting | XX | XX | | XX | XX |
| protein) (PA26) | | | | | |

*The extracellular domain of these receptors can be used as soluble protein therapeutics as antagonists of angiogenesis.

### Definitions

As used herein, the term “PA” refers to the pro-angiogenic sequences of the instant invention. The terms “PA polypeptide”, “PA protein” and “PA” when used herein encompass native sequence PA polypeptide and PA polypeptide variants (which are further defined herein). The PA polypeptide can be isolated from a variety of sources, such as human tissue types or from other sources. Additionally, they may be prepared by recombinant and/or synthetic methods. As used herein, “Pro-angiogenic (PA)” relates to the genes and gene products of the invention a differential expression of which is promoted in comparison to a reference state that does not favor angiogenesis and/or vascularization. As used herein, therefore, “pro-angiogenic” includes those genes and gene products which whose expression is enhanced in an angiogenic environment as well as those genes and gene products whose expression is suppressed in such an environment. Included among the genes and gene products that are enhanced are tissue factor pathway inhibitor-2, aggrecanase (ADAMTS-4), protein KIAA0188, placental growth factor (PIGF), fibroblast growth factor 16 (FGF-16), stanniocalcin precursor, tyrosine kinase receptor (epithelial cell, ECK), interleukin 6 signal transducer (gp130), CD82, podocalyxin-like protein, OX40 cell surface antigen, alpha-2 integrin, protein zero related protein, T-plastin, moesin, dynein light chain, myosin-IC, ankyrin-2, actin bundling protein, osteonidogen, white protein homolog, cathepsin B, and laminin gamma-2 chain. Included among the genes and gene products that are suppressed in an angiogenic environment are tyrosine kinase receptor ax1, urokinase inhibitor (PAI-2) and mesothelial keratin K7 (type II). Furthermore, additional genes or gene products differentially expressed in angiogenesis that are considered pro-angiogenic for the purposes of the invention include ALG-2 interacting protein and collagenase.

A “native sequence PA” comprises a polypeptide having the same amino acid sequence as a PA derived from nature. Such native sequence PAs can be isolated from nature or can be produced by recombinant and/or synthetic means. The term “native sequence PA” specifically encompasses naturally-occurring truncated or secreted forms (e.g., an extracellular domain sequence), naturally-occurring variant forms (e.g., alternatively spliced forms), and naturally-occurring allelic variants of the PA protein. In one embodiment of the invention, the native sequence PA is a mature or full-length native sequence PA comprising amino acids encompassing the N-terminus to the C-terminus of the known sequence.

As used herein, “pro-angiogenic” relates to the genes and gene products of the invention. Differential expression of these genes can be observed in comparison to a reference state that does not favor angiogenesis and/or vascularization. As used herein, therefore, “pro-angiogenic” includes those genes and gene products which whose expression is enhanced in an angiogenic favorable environment (i.e., an environment that promotes angiogenesis) as well as those genes and gene products whose expression is suppressed in such an environment. “PA variant polypeptide” means an active PA polypeptide as defined herein having at least about 80% amino acid sequence identity with the amino acid sequence of PA protein in Table 1. Such identity can be to the residues of the full-length polypeptide or to a specifically derived fragment of the amino acid sequence of the protein. Such PA variant polypeptides include, for instance, PA polypeptides wherein one or more amino acid residues are added, or deleted, at the N- and/or C-terminus, as well as within one or more internal domains of each amino acid sequence. Ordinarily, a PA variant polypeptide will have at least about 80% amino acid sequence identity, more preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and yet more preferably at least about 99% amino acid sequence identity with either the full-length polypeptide or a specifically derived fragment of the amino acid sequence of PA protein shown in Table 1. PA variant polypeptides do not encompass the native PA polypeptide sequence. Ordinarily, PA variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 250 amino acids in length, more often at least about 300 amino acids in length, or more.

As used herein with respect to the PA polypeptide sequences described herein, “percent (%) amino acid sequence identity” is defined as the percentage of amino acid residues in a candidate sequence that are identical with the amino acid residues in a PA sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the fill-length of the sequences being compared. For purposes herein, however, % amino acid sequence identity values are obtained as described below by using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated by one skilled in the art that where the length of amino acid sequence A is not equal to the length of amino acid sequence B, the % amino acid sequence identity of A to B will not equal the % amino acid sequence identity of B to A.

Unless specifically stated otherwise, all % amino acid sequence identity values used herein are obtained as described above using the ALIGN-2 sequence comparison computer program. However, % amino acid sequence identity can also be determined using the sequence comparison program NCBI-BLAST2 (Altschul et al., Nucleic Acids Res. 25:3389-3402 (1997)). The NCBI-BLAST2 sequence comparison program can be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“PA variant polynucleotide” or “PA variant nucleic acid sequence” means a nucleic acid molecule which encodes an active PA polypeptide, as defined herein, and which has at least about 80% nucleic acid sequence identity with either (a) a nucleic acid sequence which encodes residues comprising the known reading frame of the PA nucleic acid coding for the PA polypeptide shown in Table 2, or (b) a nucleic acid sequence which encodes another specifically derived fragment of the amino acid sequence shown in Table 1. Ordinarily, a PA variant polynucleotide will have at least about 80% nucleic acid sequence identity, more preferably at least about 81% nucleic acid sequence identity, more preferably at least about 82% nucleic acid sequence identity, more preferably at least about 83% nucleic acid sequence identity, more preferably at least about 84% nucleic acid sequence identity, more preferably at least about 85% nucleic acid sequence identity, more preferably at least about 86% nucleic acid sequence identity, more preferably at least about 87% nucleic acid sequence identity, more preferably at least about 88% nucleic acid sequence identity, more preferably at least about 89% nucleic acid sequence identity, more preferably at least about 90% nucleic acid sequence identity, more preferably at least about 91% nucleic acid sequence identity, more preferably at least about 92% nucleic acid sequence identity, more preferably at least about 93% nucleic acid sequence identity, more preferably at least about 94% nucleic acid sequence identity, more preferably at least about 95% nucleic acid sequence identity, more preferably at least about 96% nucleic acid sequence identity, more preferably at least about 97% nucleic acid sequence identity, more preferably at least about 98% nucleic acid sequence identity and yet more preferably at least about 99% nucleic acid sequence identity with either (a) a nucleic acid sequence which encodes all residues of the PA polypeptide shown in Table 1, or (b) a nucleic acid sequence which encodes another specifically derived fragment of the amino acid sequence shown in Table 1. PA polynucleotide variants do not encompass the native PA nucleotide sequence.

Ordinarily, PA variant polynucleotides are at least about 30 nucleotides in length, often at least about 60 nucleotides in length, more often at least about 90 nucleotides in length, more often at least about 120 nucleotides in length, more often at least about 150 nucleotides in length, more often at least about 180 nucleotides in length, more often at least about 210 nucleotides in length, more often at least about 240 nucleotides in length, more often at least about 270 nucleotides in length, more often at least about 300 nucleotides in length, more often at least about 450 nucleotides in length, more often at least about 600 nucleotides in length, more often at least about 900 nucleotides in length, or more.

“Percent (%) nucleic acid sequence identity” with respect to the PA polypeptide-encoding nucleic acid sequences identified herein is defined as the percentage of nucleotides in a candidate sequence that are identical with the nucleotides in a PA polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Those skilled in the art can determine appropriate parameters for measuring alignment, including any algorithms needed to achieve maximal alignment over the full-length of the sequences being compared. For purposes herein, however, % nucleic acid sequence identity values are obtained as described below by using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. The ALIGN-2 program should be compiled for use on a UNIX operating system, preferably digital UNIX V4.0D. All sequence comparison parameters are set by the ALIGN-2 program and do not vary.

For purposes herein, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

Unless specifically stated otherwise, all % nucleic acid sequence identity values used herein are obtained as described above using the ALIGN-2 sequence comparison computer program. However, % nucleic acid sequence identity can also be determined using the sequence comparison program NCBI-BLAST2 (Altschul et al., Nucleic Acids Res. 25:3389-3402 (1997)). The NCBI-BLAST2 sequence comparison program can be downloaded from http://www.ncbi.nlm.nih.gov. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows: 100 times the fraction W/Z, where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

In other embodiments, PA variant polynucleotides are nucleic acid molecules that encode an active PA polypeptide and which are capable of hybridizing, preferably under stringent hybridization and wash conditions, to nucleotide sequences encoding the full-length PA polypeptide shown in Table 1. PA variant polypeptides can be those that are encoded by a PA variant polynucleotide.

The term “positives”, in the context of the amino acid sequence identity comparisons performed as described above, includes amino acid residues in the sequences compared that are not only identical, but also those that have similar properties. Amino acid residues that score a positive value to an amino acid residue of interest are those that are either identical to the amino acid residue of interest or are a preferred substitution of the amino acid residue of interest. Preferred substitutions are shown in Table 3.

TABLE 3

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; norleucine | leu |
| Leu (L) | norleucine; ile; val; met; ala; phe | ile |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; ala; norleucine | leu |

For purposes herein, the % value of positives of a given amino acid sequence A to, with, or against a given amino acid sequence B (which can alternatively be phrased as a given amino acid sequence A that has or comprises a certain % positives to, with, or against a given amino acid sequence B) is calculated as follows: 100 times the fraction X/Y, where X is the number of amino acid residues scoring a positive value as defined above by the sequence alignment program ALIGN-2 in that program's alignment of A and B, and where Y is the total number of amino acid residues in B. It will be appreciated that where the length of amino acid sequence A is not equal to the length of amino acid sequence B, the % positives of A to B will not equal the % positives of B to A.

The term “isolated,” when used to describe the various polypeptides disclosed herein, means polypeptide that has been identified and separated and/or recovered from a component of its natural environment. Preferably, the isolated polypeptide is free of association with all components with which it is naturally associated. Contaminant components of its natural environment are materials that would typically interfere with diagnostic or therapeutic uses for the polypeptide, and can include enzymes, hormones, and other proteinaceous or non-proteinaceous solutes. In preferred embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Isolated polypeptide includes polypeptide in situ within recombinant cells, since at least one component of the PA protein natural environment will not be present. Ordinarily, however, isolated polypeptide will be prepared by at least one purification step.

An “isolated” nucleic acid molecule encoding a PA polypeptide is a nucleic acid molecule that is identified and separated from at least one contaminant nucleic acid molecule with which it is ordinarily associated in the natural source of the PA-encoding nucleic acid. Preferably, the isolated nucleic is free of association with all components with which it is naturally associated. An isolated PA-encoding nucleic acid molecule is one that is other than in the form or setting in which it is found in nature. Isolated nucleic acid molecules therefore are distinguished from PA-encoding nucleic acid molecules as they exist in natural cells. However, an isolated nucleic acid molecule encoding a PA polypeptide includes PA-encoding nucleic acid molecules contained in cells that ordinarily express PA where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Additionally, eukaryotic cells are known to utilize promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “antibody” is used in the broadest sense and specifically covers, for example, single anti-PA monoclonal antibodies (including agonist, antagonist, and neutralizing antibodies), anti-PA antibody compositions with polyepitopic specificity, single chain anti-PA polypeptide antibodies, and fragments of anti-PA polypeptide antibodies. The term “monoclonal antibody” as used herein refers to an antibody obtained from a population of substantially homogeneous antibodies, i.e., the individual antibodies comprising the population are identical except for possible naturally-occurring mutations that can be present in minor amounts.

The “stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature, which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formarnide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a PA polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues).

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin can be obtained from any immunoglobulin, such as IgG-1, IgG-2, IgG-3, or IgG-4 subtypes, IgA (including IgA-1 and IgA-2), IgE, IgD or IgM.

“Active” or “activity” for the purposes herein refers to form(s) of PA polypeptide which retain a biological and/or an immunological activity of native or naturally-occurring PA polypeptide, wherein “biological” activity refers to a biological function (either inhibitory or stimulatory), which includes enzymatic activity, caused by a native or naturally-occurring PA other than the ability to induce the production of an antibody against an antigenic epitope possessed by a native or naturally-occurring PA and an “immunological” activity refers to the ability to induce the production of an antibody against an antigenic epitope possessed by a native or naturally-occurring PA. A preferred biological activity includes, for example, the property of the PA polypeptide to degrade extracellular matrix as for example in the case of proteases discovered to do so described in this invention.

The term “antagonist” is used in the broadest sense, and includes any molecule that partially or fully blocks, inhibits, or neutralizes a biological activity of a native PA polypeptide disclosed herein. In a similar manner, the term “agonist” is used in the broadest sense and includes any molecule that mimics a biological activity of a native PA polypeptide disclosed herein. Suitable agonist or antagonist molecules specifically include agonist or antagonist antibodies or antibody fragments, fragments or amino acid sequence variants of native PA polypeptides, peptides, antisense molecules, and small organic molecules. Methods for identifying agonists or antagonists of a PA polypeptide include contacting a PA polypeptide, mRNA or gene with a candidate agonist or antagonist molecule and measuring a detectable change in one or more biological activities normally associated with the PA polypeptide.

“Treatment” refers to both therapeutic treatment and prophylactic or preventative measures, wherein the object is to prevent or slow down (lessen) the targeted pathologic condition or disorder. More specifically, “treatment” is an intervention performed with the intention of preventing the development or altering the pathology of an angiogenic disorder. The concept of treatment is used in the broadest sense, and specifically includes the prevention (prophylaxis), moderation, reduction, and curing of angiogenic disorder disorders of any stage. Accordingly, “treatment” refers to both therapeutic treatment and prophylactic or preventative measures, wherein the object is to prevent or slow down (lessen) an angiogenic disorder. The disorder may result from any cause, including idiopathic, cardiotrophic, or myotrophic causes, or ischemia or ischemic insults, such as myocardial infarction. Subjects in need of treatment include those already with the disorder as well as those susceptible to the disorder or those in whom the disorder needs to be prevented.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Microarray” refers to an array of distinct polynucleotides or oligonucleotides arranged on a substrate such as paper, nylon or other type of membrane, filter, gel, polymer, chip, glass slide, or any other suitable support, including solid supports. The polynucleotides or oligonucleotides (the backbone chemistry can be any available in the art) can be synthesized on a substrate or prepared before application to the substrate.

“Mammal” refers to any animal classified as a mammal, including humans and animals such as dogs, cats, cattle, horses, sheep, pigs, goats, rabbits, etc. Preferably, the mammal is human.

Administration “in combination with” one or more further therapeutic agents includes both simultaneous (concurrent) and consecutive administration in any order of one or more therapeutic agents.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (i.e., less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; lipids such as cationic lipids, salt-forming counterions such as sodium; and/or nonionic surfactants such as TWEEN™ polyethylene glycol (PEG), and PLURONICS™.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (See Zapata et al., Protein Ens. 8(10): 1057-1062 (1995)); single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, a designation reflecting the ability to crystallize readily. Pepsin treatment yields an F(ab′)₂ fragment that has two antigen-combining sites and is still capable of cross-linking antigen.

“Fv” is the minimum antibody fragment that contains a complete antigen-recognition and antigen-binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known to one of ordinary skill in the art.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, immunoglobulins can be assigned to different classes. There are five major classes of immunoglobulins: IgA, IgD, IgE, IgG, and IgM, and several of these can be further divided into subclasses (isotypes), e.g., IgG1, IgG2, IgG3, IgG4, IgA, and IgA2.

“Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

An “isolated” antibody is one that has been identified and separated and/or recovered from a component of its natural environment. Contaminant components of its natural environment are materials, which would interfere with diagnostic or therapeutic uses for the antibody, and can include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The word “label”, when used herein, refers to a detectable compound or composition which is conjugated directly or indirectly to the antibody so as to generate a “labeled” antibody. The label can be detectable by itself (e.g. radioisotope labels or fluorescent labels) or, in the case of an enzymatic label, can catalyze chemical alteration of a substrate compound or composition which is detectable.

By “solid phase” is meant a non-aqueous matrix to which the antibody of the present invention can adhere. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a PA polypeptide or antibody thereto) to a mammal. The components of the liposome are commonly arranged in a bilayer formation, similar to the lipid arrangement of biological membranes.

As used herein, a “small molecule” refers to any molecule having a molecular weight below about 500 Daltons.

The phrases “vascular or angiogenic disorder”, “vascular or angiogenic dysfunction” are used interchangeably and refer in part to systemic disorders that affect vessels, such as diabetes mellitus, as well as diseases of the vessels themselves, such as of the arteries, capillaries, veins, and/or lymphatics. Such diseases include indications that stimulate angiogenesis, cardiovascularization, and/or neovascularization, as well as those that inhibit angiogenesis, cardiovascularization, and/or neovascularization. Such disorders include, for example, arterial diseases, such as atherosclerosis, hypertension, inflammatory vasculitides, Reynaud's disease and Reynaud's phenomenon, aneurysms, and arterial restenosis; venous and lymphatic disorders such as thrombophlebitis, lymphangitis, and lymphedema; and other vascular disorders such as peripheral vascular disease, cancer such as vascular tumors, e.g., hemangioma (capillary and cavernous), glomus tumors, telangiectasia, bacillary angiomatosis, hemangioendothelioma, angiosarcoma, haemangiopericytoma, Kaposi's sarcoma, lymphangioma, and lymphangiosarcoma, tumor angiogenesis, trauma such as wounds, burns, and other injured tissue, implant fixation, scarring, ischemia reperfusion injury, rheumatoid arthritis, cerebrovascular disease, renal diseases such as acute renal failure, and osteoporosis. This would also include angina, myocardial infarctions such as acute myocardial infarctions and heart failure such as congestive heart failure.

The term “heart failure” refers to any abnormality of cardiac function where the heart does not pump blood at the rate needed for the requirements of metabolizing tissues. Heart failure can be caused by a number of factors, including ischemic, congenital, rheumatic, or idiopathic forms.

One type of heart failure, “congestive heart failure” (CHF), is a progressive pathologic state where the heart is increasingly unable to supply adequate cardiac output (i.e., the volume of blood pumped by the heart over time) to deliver the oxygenated blood to peripheral tissues. As CHF progresses, structural and hemodynamic damage occurs. While this damage has a variety of manifestations, one characteristic symptom is ventricular hypertrophy. CHF is a common end result of a number of various cardiac disorders.

“Myocardial infarction” generally results from atherosclerosis of the coronary arteries, often with superimposed coronary thrombosis. It may be divided into two major types: transmural infarcts, in which myocardial necrosis involves the full thickness of the ventricular wall, and subendocardial (nontransmural) infarcts, in which the necrosis involves the subendocardium, the intramural myocardium, or both, without extending all the way through the ventricular wall to the epicardium. Myocardial infarction is known to cause both a change in hemodynamic effects and an alteration in structure in the damaged and healthy zones of the heart. Therefore, for example, myocardial infarction reduces the maximum cardiac output and the stroke volume of the heart. Also associated with myocardial infarction is a stimulation of the DNA synthesis occurring in the interstice as well as an increase in the formation of collagen in the areas of the heart not affected.

Supravalvular “aortic stenosis” is an inherited vascular disorder characterized by narrowing of the ascending aorta, but other arteries, including the pulmonary arteries, may also be affected. Untreated aortic stenosis may lead to increased intracardiac pressure resulting in myocardial hypertrophy and, eventually, heart failure and death. The pathogenesis of this disorder is not fully understood, but hypertrophy and possibly hyperplasia of medial smooth muscle are prominent features of this disorder. It has been reported that molecular variants of the elastin gene are involved in the development and pathogenesis of aortic stenosis. See U.S. Pat. No. 5,650,282.

“Valvular regurgitation” occurs as a result of heart diseases resulting in disorders of the cardiac valves. Various diseases, like rheumatic fever, can cause the shrinking or pulling apart of the valve orifice, while other diseases may result in endocarditis, an inflammation of the endocardium or lining membrane of the atrioventricular orifices and operation of the heart. Defects such as the narrowing of the valve stenosis or the defective closing of the valve result in an accumulation of blood in the heart cavity or regurgitation of blood past the valve. If uncorrected, prolonged valvular stenosis or insufficiency may result in cardiac hypertrophy and associated damage to the heart muscle, which may eventually necessitate valve replacement.

Treatment of these, and other endothelial-involved cardiovascular and angiogenic disorders are encompassed by the present invention.

As used herein, the terms “cancer”, “cancerous”, and “malignant” refer to or describe the physiological condition in mammals that is typically characterized by unregulated cell growth. Examples of cancers include but are not limited to, carcinomas including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, cervical cancer, ovarian cancer, liver cancers such as hepatic carcinoma and hepatoma, bladder cancer, breast cancer, colon cancer, colorectal cancer, endometrial carcinoma, salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer. The preferred cancers for treatment according to the methods of the invention described herein are breast, colon, lung, melanoma, ovarian, and others involving vascular tumors as noted above.

The term “cytotoxic agent” as used herein refers to a substance that inhibits or prevents the function of cells and/or causes destruction of cells. The term is intended to include radioactive isotopes (e.g., ¹³¹I, ¹²⁵I, ⁹⁰Y, and ¹⁸⁶Re), chemotherapeutic agents, and toxins such as enzymatically active toxins of bacterial, fungal, plant, or animal origin, or fragments thereof.

A “chemotherapeutic agent” is a chemical compound useful in the treatment of cancer. Examples of chemotherapeutic agents include alkylating agents, folic acid antagonists, anti-metabolites of nucleic acid metabolism, antibiotics, pyrimidine analogs, 5-fluorouracil, cisplatin, purine nucleosides, amines, amino acids, triazol nucleosides, or corticosteroids. Specific examples include Adriamycin, Doxorubicin, 5-Fluorouracil, Cytosine arabinoside (“Ara-C”), Cyclophosphamide, Thiotepa, Busulfan, Cytoxin, Taxol, Toxotere, Methotrexate, Cisplatin, Melphalan, Vinblastine, Bleomycin, Etoposide, Ifosfamide, Mitomycin C, Mitoxantrone, Vincreistine, Vinorelbine, Carboplatin, Teniposide, Daunomycin, Carminomycin, Aminopterin, Dactinomycin, Mitomycins, Esperamicins (See U.S. Pat. No. 4,675,187), Melphalan, and other related nitrogen mustards. Also included in this definition are hormonal agents that act to regulate or inhibit hormone action on tumors, such as tamoxifen and onapristone.

“Growth-inhibitory agent” when used herein refers to a compound or composition that inhibits growth of a cell, such as an Wnt-overexpressing cancer cell, either in vitro or in vivo. Thus, a growth-inhibitory agent is one that significantly reduces the percentage of malignant cells in S phase. Examples of growth-inhibitory agents include agents that block cell cycle progression (at a place other than S phase), such as agents that induce G1 arrest and M-phase arrest. Classical M-phase blockers include the vincas (vincristine and vinblastine), taxol, and topo II inhibitors such as doxorubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information on growth-inhibitory agents can be found in The Molecular Basis of Cancer, Mendelsohn and Israel, eds., Chapter 1, entitled “Cell cycle regulation, oncogenes, and antineoplastic drugs” by Murakami et al., (W B Saunders: Philadelphia, 1995), p. 13. Additional examples include tumor necrosis factor (TNF), an antibody capable of inhibiting or neutralizing the angiogenic activity of acidic or basic FGF or hepatocyte growth factor (HGF), an antibody capable of inhibiting or neutralizing the coagulant activities of tissue factor, protein C, or protein S (see, WO 91/01753, published 21 Feb. 1991), or an antibody capable of binding to HER2 receptor (WO 89/06692), such as the 4D5 antibody (and functional equivalents thereof) (e.g., WO 92/22653).

A “cardiovascular agent” is a small molecule drug or a soluble protein, for example an antibody, that promotes cardiovascularization or inhibits the deterioration of existing vascular structures or protects cardiac muscle blood vessels from occlusion by thrombus plaques or other proteinaceous build up leading to the formation of a plaque in the wall of a vessel. This agent can also exert similar effects in vasculature of other tissues where artherosclerotic plaques can form.

“Angiogenic agents” and “endothelial agents” are active agents that promote angiogenesis and/or endothelial cell growth, or, if applicable, vasculogenesis. These would include factors that accelerate wound healing, such as growth hormone, insulin-like growth factor-I (IGF-I), VEGF, VIGF, PDGF, epidermal growth factor (EGF), CTGF and members of its family, FGF, and TGF-α and TGF-β.

“Angiostatic agents” are active agents that inhibit angiogenesis or vasculogenesis or otherwise inhibit or prevent growth of cancer cells. Examples include antibodies or other antagonists to angiogenic agents as defined above, such as antibodies to VEGF. They additionally include cytotherapeutic agents such as cytotoxic agents, chemotherapeutic agents, growth-inhibitory agents, apoptotic agents, and other agents to treat cancer, such as anti-HER-2, anti-CD20, and other bioactive and organic chemical agent.

In a pharmacological sense, in the context of the present invention, a “therapeutically effective amount” of an active agent such as a PA polypeptide or agonist or antagonist thereto or an anti-PA antibody, refers to an amount effective in the treatment of a cardiovascular, endothelial or angiogenic disorder in a mammal and can be determined empirically. Determination of a therapeutically effective amount can be accomplished by any method known to those skilled in the art.

As used herein, an “effective amount” of an active agent such as a PA polypeptide or agonist or antagonist thereto or an anti-PA antibody, refers to an amount effective for carrying out a stated purpose, wherein such amounts may be determined empirically for the desired effect.

### General Screening and Diagnostic Methods Using PA Sequences

Several of the herein disclosed methods relate to comparing the levels of expression of one or more PA nucleic acids in a test and reference cell populations. The sequence information disclosed herein, coupled with nucleic acid detection methods known in the art, allow for detection and comparison of the various PA transcripts. In some embodiments, the PA nucleic acids and polypeptide correspond to nucleic acids or polypeptides which include the various sequences (referenced by Genbank Accession Numbers and SEQ ID NOs) disclosed for each PA nucleic acid sequence.

In its various aspects and embodiments, the invention includes providing a test cell population which includes at least one cell that is capable of expressing one or more of the sequences PA 1-27, or any combination of PA sequences thereof. By “capable of expressing” is meant that the gene is present in an intact form in the cell and can be expressed. Expression of one, some, or all of the PA sequences is then detected, if present, and, preferably, measured. Using sequence information provided by the database entries for the known sequences, expression of the PA sequences can be detected (if expressed) and measured using techniques well known to one of ordinary skill in the art. For example, sequences within the sequence database entries corresponding to PA sequences can be used to construct probes for detecting PA RNA sequences in, e.g., northern blot hybridization analyses or methods which specifically, and, preferably, quantitatively amplify specific nucleic acid sequences. As another example, the sequences can be used to construct primers for specifically amplifying the PA sequences in, e.g., amplification-based detection methods such as reverse-transcription based polymerase chain reaction. When alterations in gene expression are associated with gene amplification or deletion, sequence comparisons in test and reference populations can be made by comparing relative amounts of the examined DNA sequences in the test and reference cell populations.

For PA sequences whose polypeptide product is known, expression can be also measured at the protein level, i.e., by measuring the levels of polypeptides encoded by the gene products described herein. Such methods are well known in the art and include, e.g., immunoassays based on antibodies to proteins encoded by the genes.

Expression level of one or more of the PA sequences in the test cell population is then compared to expression levels of the sequences in one or more cells from a reference cell population. Expression of sequences in test and control populations of cells can be compared using any art-recognized method for comparing expression of nucleic acid sequences. For example, expression can be compared using GENECALLING® methods as described in U.S. Pat. No. 5,871,697 and in Shimkets et al., Nat. Biotechnol. 17:798-803, both of which are incorporated herein by reference.

In various embodiments, the expression of 1, 2, 3, 4, 5, 6, 7, 8, 9, 10, 11, 12, 13, 14, 15, 16, 17, 18, 19, 20, 21, 22, 23, 24, 25 or all of the sequences represented by PA 1-27 are measured. If desired, expression of these sequences can be measured along with other sequences whose expression is known to be altered according to one of the herein described parameters or conditions.

The reference cell population includes one or more cells capable of expressing the measured PA sequences and for which the compared parameter is known, e.g., angiogenic stage. By “angiogenic stage” is meant that is known whether the reference cell is from a subject suffering from an angiogenic disorder. For example, a subject with a positive angiogenic stage has an angiogenic disorder whereas a subject with a negative angiogenic stage does not.

Whether or not comparison of the gene expression profile in the test cell population to the reference cell population reveals the presence, or degree, of the measured parameter depends on the composition of the reference cell population. For example, if the reference cell population is composed of cells from a subject with an angiogenic disorder (i.e., a subject with a positive angiogenic stage), a similar gene expression level in the test cell population and a reference cell population indicates the test cell population has the same positive angiogenic stage. Likewise, a different gene expression level indicates that the test cell population has a negative angiogenic stage.

In various embodiments, a PA sequence in a test cell population is considered comparable in expression level to the expression level of the PA sequence in the reference cell population if its expression level varies within a factor of less than or equal to 2.0 fold from the level of the PA transcript in the reference cell population. In various embodiments, a PA sequence in a test cell population can be considered altered in levels of expression if its expression level varies from the reference cell population by more than 2.0 fold from the expression level of the corresponding PA sequence in the reference cell population.

If desired, comparison of differentially expressed sequences between a test cell population and a reference cell population can be done with respect to a control nucleic acid whose expression is independent of the parameter or condition being measured. Expression levels of the control nucleic acid in the test and reference nucleic acid can be used to normalize signal levels in the compared populations. Suitable control nucleic acids can readily be determined by one of ordinary skill in the art.

In some embodiments, the test cell population is compared to multiple reference cell populations. Each of the multiple reference populations may differ in the known parameter. Thus, a test cell population may be compared to a first reference cell population having a positive angiogenic stage as well as a second reference population having a negative angiogenic stage.

The test cell population can be any number of cells, i.e., one or more cells, and can be provided in vitro, in vivo, or ex vivo.

In other embodiments, the test cell population can be divided into two or more sub-populations. The sub-populations can be created by dividing the first population of cells to create as identical a sub-population as possible. This will be suitable, in, for example, in vitro or ex vivo screening methods. In some embodiments, various sub-populations can be exposed to a control agent, and/or a test agent, multiple test agents, or, e.g., varying dosages of one or multiple test agents administered together, or in various combinations.

Preferably, cells in the reference cell population are derived from a tissue type as similar as possible to test cell. In some embodiments, the control cell is derived from the same subject as the test cell, e.g., from a region proximal to the region of origin of the test cell. In other embodiments, the reference cell population is derived from a plurality of cells. For example, the reference cell population can be a database of expression patterns from previously tested cells for which one of the herein-described parameters or conditions (e.g., angiogenic stage, diagnostic, or therapeutic claims) is known.

The subject is preferably a mammal. The mammal can be, e.g., a human, non-human primate, mouse, rat, dog, cat, horse, or cow.

### Methods of Determining or Diagnosing the Susceptibility to an Angiogenic Disorder

The invention further provides a method of determining or diagnosing the susceptibility to an angiogenic disorder. An angiogenic disorder is diagnosed by examining the expression of two or more PA nucleic acid sequences from a test population of cells from a subject suspected of having the disorder.

Expression of one or more of the PA nucleic acid sequences, e.g. PAs: 1-27 is measured in the test cell population and is compared to the expression of the sequences in the reference cell population. The reference cell population contains at least one cell from a subject not suffering therefrom. If the reference cell population contains cells that have a disorder, then a similarity in expression between PA sequences in the test population and the reference cell population indicates the subject is obese. A difference in expression between PA sequences in the test population and the reference cell population indicates that the subject is not obese.

The subject is preferably a mammal. The mammal can be, e.g., a human, non-human primate, mouse, rat, dog, cat, horse, or cow.

In various embodiments, the expression of 1, 2, 3, 4, 5, 6, 7, 8, 9, 10, 11, 12, 13, 14, 15, 16, 17, 18, 19, 20, 21, 22, 23, 24, 25, or all of the sequences represented by PA 1-27 are measured. If desired, expression of these sequences can be measured along with other sequences whose expression is known to be altered according to one of the herein described parameters or conditions.

### Methods of Treating an Angiogenic Disorder

Also included in the invention is a method of treating, i.e., preventing or delaying the onset of an angiogenic disorder in a subject by administering to the subject an agent which modulates the expression or activity of one or more nucleic acids selected from the group consisting of PA 1-27. “Modulates” is meant to include increased or decreased expression or activity of the PA nucleic acids. Preferably, modulation results in alteration of the expression or activity of the PA genes or gene products in a subject to a level similar or identical to a subject not suffering from the angiogenic disorder.

The subject can be, e.g., a human, a rodent such as a mouse or rat, or a dog or cat.

In one aspect, the method of treatment involves the administration of an agent that decreases the expression of one or more of the nucleic acid sequences selected from the group consisting of PAs:5, 14, and 15. Alternatively, the method can involve the administration of an agent that increases the expression of one or more nucleic acid sequence selected from the group consisting of Pas:1-4, 6-13, and 16-26.

Suitable agents may include antibodies to polypeptides encoded by the particular PA nucleic acid sequence, an antisense nucleic acid molecule, a peptide, a PA polypeptide agonist, a PA polypeptide antagonist, a peptidomimmetic, a small molecule, or other drugs.

In various embodiments, the angiogenic disorder to be treated can be selected from the group consisting of cardiac hypertrophy, trauma, age-related macular degeneration, and cancer. Other angiogenic disorders may also be treated according to the methods of the instant invention.

The herein described PA nucleic acids, polypeptides, antibodies, agonists, and antagonists, when used therapeutically are referred to herein as “Therapeutics”. Methods of administration of Therapeutics include, but are not limited to, intradermal, intramuscular, intraperitoneal, intravenous, subcutaneous, intranasal, epidural, and oral routes. The Therapeutics of the present invention may be administered by any convenient route, for example by infusion or bolus injection, by absorption through epithelial or mucocutaneous linings (e.g., oral mucosal, rectal and intestinal mucosal, etc.) and may be administered together with other biologically-active agents. Administration can be systemic or local. In addition, it may be advantageous to administer the Therapeutic into the central nervous system by any suitable route, including intraventricular and intrathecal injection.

Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant. In a specific embodiment, administration may be by direct injection at the site (or former site) of a malignant tumor or neoplastic or pre-neoplastic tissue.

Various delivery systems are known and can be used to administer a Therapeutic of the present invention including, e.g.: (i) encapsulation in liposomes, microparticles, microcapsules; (ii) recombinant cells capable of expressing the Therapeutic; (iii) receptor-mediated endocytosis (See, e.g., Wu and Wu, 1987. J Biol Chem 262:4429-4432); (iv) construction of a Therapeutic nucleic acid as part of a retroviral or other vector, and the like. In one embodiment of the present invention, the Therapeutic may be delivered in a vesicle, in particular a liposome. In a liposome, the protein of the present invention is combined, in addition to other pharmaceutically acceptable carriers, with amphipathic agents such as lipids, which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,837,028; and U.S. Pat. No. 4,737,323, all of which are incorporated herein by reference. In yet another embodiment, the Therapeutic can be delivered in a controlled release system including, e.g.: a delivery pump (See, e.g., Saudek, et al., 1989. New Engl J Med 321:574 and a semi-permeable polymeric material (See, e.g., Howard, et al., 1989. J Neurosurg 71:105). Additionally, the controlled release system can be placed in proximity of the therapeutic target (e.g., the brain), thus requiring only a fraction of the systemic dose. See, e.g., Goodson, In: Medical Applications of Controlled Release 1984. (CRC Press, Bocca Raton, Fla.).

In a specific embodiment of the present invention, where the Therapeutic is a nucleic acid encoding a protein, the Therapeutic nucleic acid may be administered in vivo to promote expression of its encoded protein, by constructing it as part of an appropriate nucleic acid expression vector and administering it so that it becomes intracellular (e.g., by use of a retroviral vector, by direct injection, by use of microparticle bombardment, by coating with lipids or cell-surface receptors or transfecting agents, or by administering it in linkage to a homeobox-like peptide which is known to enter the nucleus (See, e.g., Joliot, et al., 1991. Proc Natl Acad Sci USA 88:1864-1868), and the like. Alternatively, a nucleic acid Therapeutic can be introduced intracellularly and incorporated within host cell DNA for expression, by homologous recombination.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of the Therapeutic of the invention which will be effective in the treatment of a particular disorder or condition will depend on the nature of the disorder or condition, and may be determined by standard clinical techniques by those of average skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. However, suitable dosage ranges for intravenous administration of the Therapeutics of the present invention are generally about 20-500 micrograms (μg) of active compound per kilogram (Kg) body weight. Suitable dosage ranges for intranasal administration are generally about 0.01 pg/kg body weight to 1 mg/kg body weight. Effective doses may be extrapolated from dose-response curves derived from in vitro or animal model test systems. Suppositories generally contain active ingredient in the range of 0.5% to 10% by weight; oral formulations preferably contain 10% to 95% active ingredient. The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Polynucleotides of the present invention can also be used for gene therapy. Gene therapy refers to therapy that is performed by the administration of a specific nucleic acid to a subject. Delivery of the Therapeutic nucleic acid into a mammalian subject may be either direct (i.e., the patient is directly exposed to the nucleic acid or nucleic acid-containing vector) or indirect (i.e., cells are first transformed with the nucleic acid in vitro, then transplanted into the patient). These two approaches are known, respectively, as in vivo or ex vivo gene therapy. Polynucleotides of the invention may also be administered by other known methods for introduction of nucleic acid into a cell or organism (including, without limitation, in the form of viral vectors or naked DNA). Any of the methodologies relating to gene therapy available within the art may be used in the practice of the present invention. See e.g., Goldspiel, et al., 1993. Clin Pharm 12:488-505.

Cells may also be cultured ex vivo in the presence of therapeutic agents or proteins of the present invention in order to proliferate or to produce a desired effect on or activity in such cells. Treated cells can then be introduced in vivo for therapeutic purposes.

### Assessing the Efficacy of an Anti-Angiogenic Disorder Treatment in a Subject

The differentially expressed PA sequences identified herein also allow for the course of treatment of a pathophysiology to be monitored. In this method, a test cell population is provided from a subject undergoing treatment for an agiogenic disorder. If desired, test cell populations can be taken from the subject at various time points before, during, or after treatment. Expression of one or more of the PA sequences, e.g., PAs: 1-27, in the cell population is then measured and compared to a reference cell population which includes cells whose pathophysiologic state is known. Preferably, the reference cells have not been exposed to the treatment.

If the reference cell population contains cells not exposed to the treatment and not suffering from the disorder, then a difference in expression between PA sequences in the test population and this reference cell population indicates the treatment is not efficacious. However, a similarity in expression between PA sequences in the test cell population and the reference cell population described above indicates that the treatment is efficacious

By “efficacious” is meant that the treatment leads to a decrease in the pathophysiology in a subject. When treatment is applied prophylactically, “efficacious” means that the treatment retards or prevents a pathophysiology. For example, if the anti-angiogenic disorder treatment is “efficacious”, it improves the angiogenic disorder in the subject.

Efficacy can be determined in association with any known method for treating the particular pathophysiology.

The subject is preferably a mammal. The mammal can be, e.g., a human, non-human primate, mouse, rat, dog, cat, horse, or cow.

In various embodiments, the expression of 1, 2, 3, 4, 5, 6, 7, 8, 9, 10, 11, 12, 13, 14, 15, 16, 17, 18, 19, 20, 21, 22, 23, 24, 25 or all of the sequences represented by PA 1-27 are measured. If desired, expression of these sequences can be measured along with other sequences whose expression is known to be altered according to one of the herein described parameters or conditions.

In various embodiments, the expression of the nucleic acid sequences in the test cell population may be either increased or decreased as compared to the reference cell population.

The test cell population can be any number of cells, i.e., one or more cells can be provided in vitro, in vivo, or ex vivo.

### Identifying Agents to Treat an Angiogenic Disorder

Also included in the invention are methods of identifying agents that treat an angiogenic disorder. One method includes contacting one or more PA polypeptides with a test agent and detecting a complex between the test agent and the polypeptide. A presence of a complex indicates that the test agent treats an angiogenic disorder. Absence of a complex indicates that the test agent does not treat an angiogenic disorder.

By “treat an angiogenic disorder” is meant that the test agent either increases or decreases the expression of one or more of the PA nucleic acid sequences.

A test agent can be, e.g. antibodies to the polypeptides encoded by PAs: 1-27, an antisense nucleic acid molecule, peptides, a PA polypeptide agonist, a PA polypeptide antagonist, peptidomimetics, small molecules or other drugs.

The test agent may be a known or an unknown therapeutic agent.

The angiogenic disorder to be treated may be selected from the group consisting of vascular tumors, proliferative vitreoretinopathy, rheumatoid arthritis, Crohn's disease, atherosclerosis, ovarian hyperstimulation, psoriasis, endometriosis associated with new vascularization, restensosis subsequent to balloon angioplasty, scar tissue overproduction, peripheral vascular disease, hypertension, inflammatory vasculitides, Reynaud's disease or Reynaud's phenomenon, aneurysms, arterial restensosis, thrombophlebitis, lymphangitis, lymphedema, wound healing and tissue repair, ischemia reperfusion injury, angina, myocardial infarctions, chronic heart conditions, heart failure such as congestive heart failure, age-related macular degeneration, and osteoporosis. Other angiogenic disorders may also be treated according to the methods of the instant invention.

The subject is preferably a mammal. The mammal can be, e.g., a human, a non-human primate, mouse, rat, dog, cat, horse, or cow.

### Methods of Modulating the Activity of PA Proteins

The invention provides a method for identifying modulators, i.e., candidate or test compounds or agents (e.g., antibodies to the polypeptides encoded by PAs: 1-27, an antisense nucleic acid molecule, peptides, a PA polypeptide agonist, a PA polypeptide antagonist, peptidomimetics, small molecules or other drugs) that bind to PA proteins or have a stimulatory or inhibitory effect on, for example, PA expression or PA activity.

In one embodiment, the invention provides assays for screening candidate or test compounds which bind to or modulate the activity of the membrane-bound form of a PA protein or polypeptide or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des 12:145).

Examples of methods for the synthesis of molecular libraries can be found in the art, for example in: DeWitt et al. (1993) Proc Natl Acad Sci U.S.A. 90:6909; Erb et al. (1994) Proc Natl Acad Sci U.S.A. 91:11422; Zuckermann et al. (1994) J Med Chem 37:2678; Cho et al. (1993) Science 261:1303; Carrell et al. (1994) Angew Chem Int Ed Engl 33:2059; Carell et al. (1994) Angew Chem Int Ed Engl 33:2061; and Gallop et al. (1994) J Med Chem 37:1233.

Libraries of compounds may be presented in solution (e.g., Houghten (1992) Biotechniques 13:412-421), or on beads (Lam (1991) Nature 354:82-84), on chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390; Devlin (1990) Science 249:404-406; Cwirla et al. (1990) Proc Natl Acad Sci U.S.A. 87:6378-6382; Felici (1991) J Mol Biol 222:301-310; Ladner above.).

In one embodiment, an assay is a cell-based assay in which a cell which expresses a membrane-bound form of PA protein, or a biologically active portion thereof, on the cell surface is contacted with a test compound and the ability of the test compound to bind to a PA protein determined. The cell, for example, can be of mammalian origin or a yeast cell. Determining the ability of the test compound to bind to the PA protein can be accomplished, for example, by coupling the test compound with a radioisotope or enzymatic label such that binding of the test compound to the PA protein or biologically active portion thereof can be determined by detecting the labeled compound in a complex. For example, test compounds can be labeled with 125I, 35S, 14C, or 3H, either directly or indirectly, and the radioisotope detected by direct counting of radioemission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of PA protein, or a biologically active portion thereof, on the cell surface with a known compound which binds PA to form an assay mixture, contacting the assay mixture with a test compound, and determining the ability of the test compound to interact with a PA protein, wherein determining the ability of the test compound to interact with a PA protein comprises determining the ability of the test compound to preferentially bind to PA or a biologically active portion thereof as compared to the known compound.

In another embodiment, an assay is a cell-based assay comprising contacting a cell expressing a membrane-bound form of PA protein, or a biologically active portion thereof, on the cell surface with a test compound and determining the ability of the test compound to modulate (e.g., stimulate or inhibit) the activity of the PA protein or biologically active portion thereof. Determining the ability of the test compound to modulate the activity of PA or a biologically active portion thereof can be accomplished, for example, by determining the ability of the PA protein to bind to or interact with a PA target molecule. As used herein, a “target molecule” is a molecule with which a PA protein binds or interacts in nature, for example, a molecule on the surface of a cell which expresses a PA interacting protein, a molecule on the surface of a second cell, a molecule in the extracellular milieu, a molecule associated with the internal surface of a cell membrane or a cytoplasmic molecule. An PA target molecule can be a non-PA molecule or a PA protein or polypeptide of the present invention. In one embodiment, a PA target molecule is a component of a signal transduction pathway that facilitates transduction of an extracellular signal (e.g. a signal generated by binding of a compound to a membrane-bound PA molecule) through the cell membrane and into the cell. The target, for example, can be a second intercellular protein that has catalytic activity or a protein that facilitates the association of downstream signaling molecules with PA.

Determining the ability of the PA protein to bind to or interact with a PA target molecule can be accomplished by one of the methods described above for determining direct binding. In one embodiment, determining the ability of the PA protein to bind to or interact with a PA target molecule can be accomplished by determining the activity of the target molecule. For example, the activity of the target molecule can be determined by detecting induction of a cellular second messenger of the target (i.e. intracellular Ca2+, diacylglycerol, IP3, etc.), detecting catalytic/enzymatic activity of the target an appropriate substrate, detecting the induction of a reporter gene (comprising a PA-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the present invention is a cell-free assay comprising contacting a PA protein or biologically active portion thereof with a test compound and determining the ability of the test compound to bind to the PA protein or biologically active portion thereof. Binding of the test compound to the PA protein can be determined either directly or indirectly as described above. In one embodiment, the assay comprises contacting the PA protein or biologically active portion thereof with a known compound which binds PA to form an assay mixture, contacting the assay mixture with a test compound, and determining the ability of the test compound to interact with a PA protein, wherein determining the ability of the test compound to interact with a PA protein comprises determining the ability of the test compound to preferentially bind to PA or biologically active portion thereof as compared to the known compound.

In another embodiment, an assay is a cell-free assay comprising contacting PA protein or biologically active portion thereof with a test compound and determining the ability of the test compound to modulate (e.g. stimulate or inhibit) the activity of the PA protein or biologically active portion thereof. Determining the ability of the test compound to modulate the activity of PA can be accomplished, for example, by determining the ability of the PA protein to bind to a PA target molecule by one of the methods described above for determining direct binding. In an alternative embodiment, determining the ability of the test compound to modulate the activity of PA can be accomplished by determining the ability of the PA protein further modulate a PA target molecule. For example, the catalytic/enzymatic activity of the target molecule on an appropriate substrate can be determined as previously described.

In yet another embodiment, the cell-free assay comprises contacting the PA protein or biologically active portion thereof with a known compound which binds PA to form an assay mixture, contacting the assay mixture with a test compound, and determining the ability of the test compound to interact with a PA protein, wherein determining the ability of the test compound to interact with a PA protein comprises determining the ability of the PA protein to preferentially bind to or modulate the activity of a PA target molecule.

The cell-free assays of the present invention are amenable to use of both the soluble form or the membrane-bound form of PA. In the case of cell-free assays comprising the membrane-bound form of PA, it may be desirable to utilize a solubilizing agent such that the membrane-bound form of PA is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)n, N-dodecyl-N,N-dimethyl-3-ammonio-1-propane sulfonate, 3-(3-cholamidopropyl)dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the present invention, it may be desirable to immobilize either PA or its target molecule to facilitate separation of complexed from uncomplexed forms of one or both of the proteins, as well as to accommodate automation of the assay. Binding of a test compound to PA, or interaction of PA with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-PA fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or PA protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described above. Alternatively, the complexes can be dissociated from the matrix, and the level of PA binding or activity determined using standard techniques.

Other techniques for immobilizing proteins on matrices can also be used in the screening assays of the invention. For example, either PA or its target molecule can be immobilized utilizing conjugation of biotin and streptavidin. Biotinylated PA or target molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with PA or target molecules, but which do not interfere with binding of the PA protein to its target molecule, can be derivatized to the wells of the plate, and unbound target or PA trapped in the wells by antibody conjugation. Methods for detecting such complexes, in addition to those described above for the GST-immobilized complexes, include immunodetection of complexes using antibodies reactive with the PA or target molecule, as well as enzyme-linked assays that rely on detecting an enzymatic activity associated with the PA or target molecule.

In another embodiment, modulators of PA expression are identified in a method wherein a cell is contacted with a candidate compound and the expression of PA mRNA or protein in the cell is determined. The level of expression of PA mRNA or protein in the presence of the candidate compound is compared to the level of expression of PA mRNA or protein in the absence of the candidate compound. The candidate compound can then be identified as a modulator of PA expression based on this comparison. For example, when expression of PA mRNA or protein is greater (statistically significantly greater) in the presence of the candidate compound than in its absence, the candidate compound is identified as a stimulator of PA mRNA or protein expression. Alternatively, when expression of PA mRNA or protein is less (statistically significantly less) in the presence of the candidate compound than in its absence, the candidate compound is identified as an inhibitor of PA mRNA or protein expression. The level of PA mRNA or protein expression in the cells can be determined by methods described herein for detecting PA mRNA or protein.

In yet another aspect of the invention, the PA proteins can be used as “bait proteins” in a two-hybrid assay or three hybrid assay (see, e.g., U.S. Pat. No. 5,283,317; Zervos et al. (1993) Cell 72:223-232; Madura et al. (1993) J Biol Chem 268:12046-12054; Bartel et al. (1993) Biotechniques 14:920-924; Iwabuchi et al. (1993) Oncogene 8:1693-1696; and Brent WO94/10300), to identify other proteins that bind to or interact with PA (“PA-binding proteins” or “PA-bp”) and modulate PA activity. Such PA-binding proteins are also likely to be involved in the propagation of signals by the PA proteins as, for example, upstream or downstream elements of the PA pathway.

The two-hybrid system is based on the modular nature of most transcription factors, which consist of separable DNA-binding and activation domains. Briefly, the assay utilizes two different DNA constructs. In one construct, the gene that codes for PA is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a PA-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein, which interacts with PA.

### Methods of Detecting PA Proteins

The invention also provides a method for detecting the presence or absence of PA in a biological sample. The method includes obtaining a biological sample from a test subject and contacting the biological sample with a compound or an agent capable of detecting PA protein or nucleic acid (e.g., mRNA, genomic DNA) that encodes PA protein such that the presence of PA is detected in the biological sample. An agent for detecting PA mRNA or genomic DNA is a labeled nucleic acid probe capable of hybridizing to PA mRNA or genomic DNA. The nucleic acid probe can be, for example, a full-length PA nucleic acid, such as the nucleic acid of SEQ ID NO:1, or a portion thereof, such as an oligonucleotide of at least 15, 30, 50, 100, 250 or 500 nucleotides in length and sufficient to specifically hybridize under stringent conditions to PA mRNA or genomic DNA. Other suitable probes for use in the diagnostic assays of the invention are described herein.

An agent for detecting PA protein is an antibody capable of binding to PA protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect PA mRNA, protein, or genomic DNA in a biological sample in vitro as well as in vivo. For example, in vitro techniques for detection of PA mRNA include Northern hybridizations and in situ hybridizations. In vitro techniques for detection of PA protein include enzyme linked immunosorbent assays (ELISAs), Western blots, immunoprecipitations and immunofluorescence. In vitro techniques for detection of PA genomic DNA include Southern hybridizations. Furthermore, in vivo techniques for detection of PA protein include introducing into a subject a labeled anti-PA antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject.

In another embodiment, the methods further involve obtaining a control biological sample from a control subject, contacting the control sample with a compound or agent capable of detecting PA protein, mRNA, or genomic DNA, such that the presence of PA protein, mRNA or genomic DNA is detected in the biological sample, and comparing the presence of PA protein, mRNA or genomic DNA in the control sample with the presence of PA protein, mRNA or genomic DNA in the test sample.

### PA Polypeptide Variants

In addition to the full-length native sequence PA polypeptides described herein, PA variants with functions similar to those of the PA polypeptides disclosed can be prepared. PA variants can be prepared by introducing appropriate nucleotide changes into the PA DNA, and/or by synthesis of the desired PA polypeptide. Those skilled in the art will appreciate that amino acid changes can alter post-translational processing of the PA, such as changing the number or position of glycosylation sites or altering the membrane anchoring characteristics.

Variations in the native full-length sequence PA or in various domains of the PA described herein, can be made, for example, using any of the techniques and guidelines for conservative and non-conservative mutations set forth, for instance, in U.S. Pat. No. 5,364,934. Variations can include the substitution, deletion or insertion of one or more codons encoding the PA that results in a change in the amino acid sequence of the PA as compared with the native sequence PA. Optionally, the variation is by substitution of at least one amino acid with any other amino acid in one or more of the domains of the PA polypeptides. Guidance in determining which amino acid residue can be inserted, substituted or deleted without adversely affecting the desired activity can be found by comparing the sequence of the PA polypeptide with that of homologous known protein derived from other mammals and minimizing the number of amino acid sequence changes made in regions of high homology. Amino acid substitutions can be the result of replacing one amino acid with another amino acid having similar structural and/or chemical properties, such as the replacement of a leucine with a serine. Such substitutions are known as conservative amino acid replacements. Insertions or deletions are, optionally, in the range of about 1 to 5 amino acids. The variation allowed can be determined by systematically making insertions, deletions or substitutions of amino acids in the sequence and testing the resulting variants for activity exhibited by the full-length or mature native sequence.

PA fragments can be prepared by any of a number of conventional techniques. Desired peptide fragments can also be chemically synthesized. An alternative approach involves generating PA fragments by enzymatic digestion, e.g., by treating the protein with an enzyme known to cleave proteins at sites defined by particular amino acid residues, or by digesting the DNA with suitable restriction enzymes and isolating the desired fragment. Another suitable technique involves isolating and amplifying a DNA fragment encoding a desired polypeptide fragment, by polymerase chain reaction (PCR). Oligonucleotides that define the desired termini of the DNA fragment are employed at the 5′ and 3′ primers in the PCR. Preferably, PA polypeptide fragments share at least one biological and/or immunological activity with the native PA polypeptide.

In particular embodiments, conservative substitutions of interest are shown in Table 3 under the heading of preferred substitutions. If such conservative substitutions result in a change in biological activity, then more substantial changes, denominated exemplary substitutions in Table 3, or as further described below in reference to amino acid classes, are introduced and the products screened for activity.

Substantial modifications in function or immunological identity of the PA polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues can be divided into groups based on common side-chain properties: (1) hydrophobic: norleucine, met, ala, vat, leu, ile; (2) neutral hydrophilic: cys, ser, thr; (3) acidic: asp, glu; (4) basic: asn, gin, his, lys, arg; (5) residues that influence chain orientation: gly, pro; and (6) aromatic: trp, tyr, phe.

Non-conservative substitutions will entail exchanging a member of one of these classes for another class. Such substituted residues also can be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis (See Carter et al., Nucl. Acids Res., 13:4331 (1986); Zoller et al., Nucl. Acids Res., 10:6487 (1987)), cassette mutagenesis (See Wells et al., Gene, 34:315 (1985)), restriction selection mutagenesis (See Wells et al., Philos. Trans. R. Soc. London SerA, 317:415 (1986)) or any other known techniques can be performed on the cloned DNA to produce the PA variant DNA.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and, thus, is less likely to alter the main-chain conformation of the variant (See Cunningham and Wells, Science, 244: 1081-1085 (1989)). Alanine is also typically preferred because it is the most common amino acid. Further, it is frequently found in both buried and exposed positions (See Creighton, The Proteins, (W. H. Freeman & Co., N.Y.); Chothia, J. Mol. Biol., 150:1 (1976)). However, if alanine substitution does not yield adequate amounts of variant, an isoteric amino acid can be used.

Modifications of PA Polypeptides

Covalent modifications of PA are included within the scope of this invention. One type of covalent modification includes reacting targeted amino acid residues of a PA polypeptide with an organic derivatizing agent that is capable of reacting with selected side chains or the N- or C-terminal residues of the PA. Derivatization with bifunctional agents is useful, for instance, for crosslinking PA to a water-insoluble support matrix or surface for use in the method for purifying anti-PA antibodies, and vice-versa. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the -amino groups of lysine, arginine, and histidine side chains (See T. E. Creighton, Proteins: Structure and Molecular Properties, W. H. Freeman & Co., San Francisco, pp. 79-86 (1983)), acetylation of the N-terminal amine, and amidation of any C-terminal carboxyl group.

Another type of covalent modification of the PA polypeptide included within the scope of this invention comprises altering the native glycosylation pattern of the polypeptide. “Altering the native glycosylation pattern” is intended for purposes herein to mean deleting one or more carbohydrate moieties found in native sequence PA (either by removing the underlying glycosylation site or by deleting the glycosylation by chemical and/or enzymatic means), and/or adding one or more glycosylation sites that are not present in the native sequence PA. In addition, the phrase includes qualitative changes in the glycosylation of the native proteins, involving a change in the nature and proportions of the various carbohydrate moieties present.

Addition of glycosylation sites to the PA polypeptide can be accomplished by altering the 30 amino acid sequence. The alteration can be made, for example, by the addition of, or substitution by, one or more serine or threonine residues to the native sequence PA (for O-linked glycosylation sites). The PA amino acid sequence can optionally be altered through changes at the DNA level, particularly by mutating the DNA encoding the PA polypeptide at preselected bases such that codons are generated that will translate into the desired amino acids.

Another means of increasing the number of carbohydrate moieties on the PA polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on the PA polypeptide can be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of PA comprises linking the PA polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The PA of the present invention can also be modified in a way to form a chimeric molecule comprising PA fused to another, heterologous polypeptide or amino acid sequence.

In one embodiment, such a chimeric molecule comprises a fusion of the PA with a tag polypeptide, which provides an epitope to which an anti-tag antibody can selectively bind. The epitope tag is generally placed at the amino- or carboxyl-terminus of the PA. The presence of such epitope-tagged forms of the PA can be detected using an antibody against the tag polypeptide. Also, provision of the epitope tag enables the PA to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 (Field et al., Mol. Cell. Biol., 8:2159-2165 (1988)); the c-myc tag and the 8F9, 3C7, 6E10, G4, B7 and 9E10 antibodies thereto (Evan et al., Molecular and Cellular Biology, 5:3610-3616 (1985)); and the Herpes Simplex virus glycoprotein D (gD) tag and its antibody (Paborsky et al., Protein Engineering, 3(6):547-553 (1990)). Other tag polypeptides include the Flag-peptide (Hopp et al., Bio Technology, 6:1204-1210 (1988)); the KT3 epitope peptide (Martin et al., Science, 255:192-194 (1992)); an α-tubulin epitope peptide (Skinner et al., J. Biol. Chem., 266:15163-15166 (1991)); and the T7 gene 10 protein peptide tag (Lutz-Freyermuth et al., Proc. Natl. Acad. Sci. USA, 87:6393-6397 (1990)).

In an alternative embodiment, the chimeric molecule can comprise a fusion of the PA polypeptide with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a PA polypeptide in place of at least one variable region within an Ig molecule. In a particularly preferred embodiment, the immunoglobulin fusion includes the hinge, CH2 and CH3, or the hinge, CH1, CH2 and CH3 regions of an IgG1 molecule. For the production of immunoglobulin fusions see also U.S. Pat. No. 5,428,130 issued Jun. 27, 1995.

In another embodiment, the chimeric molecule includes a fusion of a PA with a signal peptide to allow or enhance secretion of the PA peptide or even to change its localization within the host cell. The signal sequence is generally placed at the amino- or carboxyl-terminus of the PA, more usually the N-terminus when secretion or membrane localization is desired. Such fusions are typically intermediate products, since the signal peptide is usually specifically cleaved by enzymes of the host cell. Provision of a signal peptide enables the PA to be readily purified following its secretion to the culture medium. Various signal polypeptides, which allow secretion or targeting to compartments within the cell, are well known in the art and are available for use with numerous host cells, including yeast and mammalian cells.

### Detecting Gene Amplification/Expression

Gene amplification and/or expression can be measured in a sample directly, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. Alternatively, antibodies can be employed that can recognize specific duplexes, including DNA duplexes, RNA duplexes, and DNA-RNA hybrid duplexes or DNA-protein duplexes. The antibodies in turn can be labeled and the assay can be carried out where the duplex is bound to a surface, so that upon the formation of duplex on the surface, the presence of antibody bound to the duplex can be detected.

Gene expression, alternatively, can be measured by immunological methods, such as immunohistochemical staining of cells or tissue sections and assay of cell culture or body fluids, to quantitate directly the expression of gene product. Antibodies useful for immunohistochemical staining and/or assay of sample fluids can be either monoclonal or polyclonal, and can be prepared in any mammal. Conveniently, the antibodies can be prepared against a native sequence PA polypeptide or against a synthetic peptide based on the DNA sequences provided herein or against exogenous sequence fused to PA DNA and encoding a specific antibody epitope.

### Purification of Polypeptide

Forms of PA can be recovered from culture medium or from host cell lysates. If membrane-bound, it can be released from the membrane using a suitable detergent solution (e.g. Triton-X 100) or by enzymatic cleavage. Cells employed in expression of PA polypeptide can be disrupted by various physical or chemical means, such as freeze-thaw cycling, sonication, mechanical disruption, or cell lysing agents.

It can be desired to purify PA from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the PA. Additionally, other purification methods known to those skilled in the art can be used. Various methods of protein purification can be employed and such methods are known in the art and described for example in Deutscher, Methods in Entomology, 182 (1990); Scopes, Protein Purification: Principles and Practice, Springer-Verlag, New York (1982). The purification step(s) selected will depend, for example, on the nature of the production process used and the particular PA produced.

### Uses for PA Polypeptides

When the coding sequences for a PA polypeptide encode a protein which binds to another protein, the PA polypeptide can be used in assays to identify the other proteins or molecules involved in the binding interaction. By such methods, inhibitors of the binding interaction can be identified. Proteins involved in such binding interactions can also be used to screen for peptide or small molecule inhibitors or agonists of the binding interaction. Also, the receptor PA can be used to isolate correlative ligand(s). Screening assays can be designed to find lead compounds that mimic the biological activity of a native PA or a receptor for PA. Such screening assays will include assays amenable to high-throughput screening of chemical libraries, making them particularly suitable for identifying small molecule drug candidates. Small molecules contemplated include both synthetic organic or inorganic compounds. The assays can be performed in a variety of formats, including protein-protein binding assays, biochemical screening assays, immunoassays and cell based assays, which are well characterized in the art. Such high- and ultra-high throughput assays are can also be used to test antisense molecules.

Nucleic acids which encode PA polypeptide, or its modified forms, can also be used to generate either transgenic animals or “knock out” animals which, in turn, are useful in the development and screening of therapeutically useful reagents. A transgenic animal (e.g., a mouse or rat) is an animal having cells that contain a transgene, which transgene was introduced into the animal or an ancestor of the animal at a prenatal, e.g., an embryonic stage. A transgene is a DNA which is integrated into the genome of a cell from which a transgenic animal develops. In one embodiment, cDNA encoding PA can be used to clone genomic DNA encoding PA in accordance with established techniques and the genomic sequences used to generate transgenic animals that contain cells which express DNA encoding PA. Methods for generating transgenic animals, particularly animals such as mice or rats, have become conventional in the art and are described, for example, in U.S. Pat. Nos. 4,736,866 and 4,870,009. Typically, particular cells would be targeted for PA transgene incorporation with tissue-specific enhancers. Transgenic animals that include a copy of a transgene encoding PA introduced into the germ line of the animal at an embryonic stage can be used to examine the effect of increased expression of DNA encoding PA polypeptide. Such animals can be used as tester animals for reagents thought to confer protection from, for example, pathological conditions associated with its overexpression.

In accordance with this facet of the invention, an animal is treated with the reagent and a reduced incidence of the pathological condition, compared to untreated animals bearing the transgene, would indicate a potential therapeutic intervention for the pathological condition.

Alternatively, non-human homologues of PA gene can be used to construct a PA gene “knock out” animal which has a defective or altered gene encoding a PA polypeptide as a result of homologous recombination between the endogenous gene encoding a PA polypeptide and altered genomic DNA encoding a PA polypeptide introduced into an embryonic stem cell of the animal. For example, cDNA encoding PA polypeptides can be used to clone genomic DNA encoding PA in accordance with established techniques. A portion of the genomic DNA encoding PA can be deleted or replaced with another gene, such as a gene encoding a selectable marker which can be used to monitor integration. Typically, several kilobases of unaltered flanking DNA (both at the 5′ and 3′ ends) are included in the vector (See e.g., Thomas and Capecchi, Cell, 51:503 (1987) for a description of homologous recombination vectors). The vector is introduced into an embryonic stem cell line (e.g., by electroporation) and cells in which the introduced DNA has homologously recombined with the endogenous DNA are selected (See e.g., Li et al., Cell, 69:915 (1992)). The selected cells are then injected into a blastocyst of an animal (e.g., a mouse or rat) to form aggregation chimeras (See e.g., Bradley, in Teratocarcinomas and Embryonic Stem Cells: A Practical Approach, E. J. Robertson, ed. (IRL, Oxford, 1987), pp. 113-152). A chimeric embryo can then be implanted into a suitable pseudopregnant female foster animal and the embryo brought to term to create a “knock out” animal. Progeny harboring the homologously recombined DNA in their germ cells can be identified by standard techniques and used to breed animals in which all cells of the animal contain the homologously recombined DNA. Knockout animals can be characterized for instance, for their ability to defend against certain pathological conditions and for their development of pathological conditions due to absence of the PA polypeptide.

### Assays for Angiogenic Activity

Various assays can be used to test the polypeptide herein for angiogenic activity. Such assays include those provided in the Examples below.

Assays for tissue generation activity include, without limitation, those described in WO 95/16035 (bone, cartilage, tendon); WO 95/05846 (nerve, neuronal), and WO 91/07491 (skin, endothelium).

Assays for wound-healing activity include, for example, those described in Winter, Epidermal Wound Healing, Maibach, H I and Rovee, D T, eds. (Year Book Medical Publishers, Inc., Chicago), pp. 71-112, as modified by the article of Eaglstein and Mertz, J. Invest. Dermatol., 71: 382-384 (1978).

### Cell-Based Assays

Cell-based assays and animal models for angiogenic disorders, such as tumors, can be used to verify the findings of an angiogenic assay herein, and further to understand the relationship between the genes identified herein and the development and pathogenesis of undesirable angiogenic cell growth. The role of gene products identified herein in the development and pathology of desirable or undesirable angiogenic cell growth (e.g., endothelial cells, tumor cells) can be tested by using cells or cells lines that have been identified as being stimulated or inhibited by the PA polypeptide, or its agonists or antagonists, herein. Such cells include, for example, those set forth in the Examples below.

In a different approach, cells of a cell type known to be involved in a particular angiogenic activity or disorder are transfected with the cDNAs herein, and the ability of these cDNAs to induce excessive growth or inhibit growth is analyzed. If the angiogenic disorder is cancer, suitable tumor cells include, for example, stable tumor cells lines such as the B104-1-1 cell line (stable NIH-3T3 cell line transfected with the neu protooncogene) and ras-transfected NIH-3T3 cells, which can be transfected with the desired gene and monitored for tumorigenic growth. Such transfected cell lines can then be used to test the ability of poly- or monoclonal antibodies or antibody compositions to inhibit tumorigenic cell growth by exerting cytostatic or cytotoxic activity on the growth of the transformed cells, or by mediating antibody-dependent cellular cytotoxicity (ADCC). Cells transfected with the coding sequences of the genes identified herein can further be used to identify drug candidates for the treatment of angiogenic disorders such as cancer.

In another assays, human umbilical cord endothelial cells (HUVECS) undergoing tube formation in collagen gels in the presence of growth factors, mimic the angiogenic environment of endothelial cells in vivo, providing a well-accepted system for angiogenesis and vasculogenesis, both in normal and neoplastic conditions. The three dimensional gel is a prerequisite for the differentiation and fusion of endothelial cells into tubes, since HUVECS grown on the surface of gelatin gels or on plastic do not undergo tube-formation. HUVECS can be grown under various conditions, including inductive or non-inductive to tube formation, either on gelatin or collagen film (non-inductive) or in collagen gels (inductive), with or without the addition of growth factors to simulate normal angiogenic- or tumor-derived factors. HUVEC cells can be transfected with the cDNAs described herein (or their antisense), and the ability of these nucleic acids to induce excessive growth or tube formation or inhibit growth or tube formation is analyzed. HUVEC cells expressing coding sequences of the genes identified herein can further be used to identify drug candidates.

In addition, primary cultures derived from tumors in transgenic animals (as described above) can be used in the cell-based assays herein, although stable cell lines are preferred. Techniques to derive continuous cell lines from transgenic animals are well known in the art. See, e.g., Small et al., Mol. Cell. Biol., 5: 642-648 (1985).

For cancer, a variety of well-known animal models can be used to further understand the role of the genes identified herein in the development and pathogenesis of tumors, and to test the efficacy of candidate therapeutic agents, including antibodies and other antagonists of the native PA polypeptides, such as small-molecule antagonists. The in vivo nature of such models makes them particularly predictive of responses in human patients. Animal models of tumors and cancers (e.g., breast cancer, colon cancer, prostate cancer, lung cancer, etc.) include both non-recombinant and recombinant (transgenic) animals. Non-recombinant animal models include, for example, rodent, e.g., murine models. Such models can be generated by introducing tumor cells into syngeneic mice using standard techniques, e.g., subcutaneous injection, tail vein injection, spleen implantation, intraperitoneal implantation, implantation under the renal capsule, or orthopin implantation, e.g., colon cancer cells implanted in colonic tissue. See, e.g., PCT publication No. WO 97/33551, published Sep. 18, 1997. Probably the most often used animal species in oncological studies are immunodeficient mice and, in particular, nude mice. The observation that the nude mouse with thymic hypo/aplasia could successfully act as a host for human tumor xenografts has lead to its widespread use for this purpose. The autosomal recessive nu gene has been introduced into a very large number of distinct congenic strains of nude mouse, including, for example, ASW, A/He, AKR, BALB/c, B10.LP, C17, C3H, C57BL, C57, CBA, DBA, DDD, I/st, NC, NFR, NFS, NFS/N, NZB, NZC, NZW, P, RIII, and SJL. In addition, a wide variety of other animals with inherited immunological defects other than the nude mouse have been bred and used as recipients of tumor xenografts. For further details see, e.g., The Nude Mouse in Oncology Research, E. Boven and B. Winograd, eds. (CRC Press, Inc., 1991).

The cells introduced into such animals can be derived from known tumor/cancer cell lines, such as any of the above-listed tumor cell lines, and, for example, the B104-1-1 cell line (stable NIH-3T3 cell line transfected with the neu protooncogene); ras-transfected NIH-3T3 cells; Caco-2 (ATCC HTB-37); or a moderately well-differentiated grade II human colon adenocarcinoma cell line, HT-29 (ATCC HTB-38); or from tumors and cancers. Samples of tumor or cancer cells can be obtained from patients undergoing surgery, using standard conditions involving freezing and storing in liquid nitrogen. Karmali et al, Br. J. Cancer, 48: 689-696 (1983).

Tumor cells can be introduced into animals such as nude mice by a variety of procedures. The subcutaneous (s.c.) space in mice is very suitable for tumor implantation. Tumors can be transplanted s.c. as solid blocks, as needle biopsies by use of a trochar, or as cell suspensions. For solid-block or trochar implantation, tumor tissue fragments of suitable size are introduced into the s.c. space. Cell suspensions are freshly prepared from primary tumors or stable tumor cell lines, and injected subcutaneously. Tumor cells can also be injected as subdermal implants. In this location, the inoculum is deposited between the lower part of the dermal connective tissue and the s.c. tissue.

Animal models of breast cancer can be generated, for example, by implanting rat neuroblastoma cells (from which the neu oncogene was initially isolated), or neu-transformed NIH-3T3 cells into nude mice, essentially as described by Drebin et al., Proc. Nat. Acad. Sci. USA, 83: 9129-9133 (1986).

Similarly, animal models of colon cancer can be generated by passaging colon cancer cells in animals, e.g., nude mice, leading to the appearance of tumors in these animals. An orthotopic transplant model of human colon cancer in nude mice has been described, for example, by Wang et al., Cancer Research, 54: 4726-4728 (1994) and Too et al., Cancer Research, 55: 681-684 (1995). This model is based on the so-called “METAMOUSE™” sold by AntiCancer, Inc., (San Diego, Calif.).

Tumors that arise in animals can be removed and cultured in vitro. Cells from the in vitro cultures can then be passaged to animals. Such tumors can serve as targets for further testing or drug screening. Alternatively, the tumors resulting from the passage can be isolated and RNA from pre-passage cells and cells isolated after one or more rounds of passage analyzed for differential expression of genes of interest. Such passaging techniques can be performed with any known tumor or cancer cell lines.

For example, Meth A, CMS4, CMS5, CMS21. and WEHI-164 are chemically induced fibrosarcomas of BALB/c female mice (DeLeo et al., J. Exp. Med., 146: 720 (1977)), which provide a highly controllable model system for studying the anti-tumor activities of various agents. Palladino et al., J. Immunol., 138: 4023-4032 (1987). Briefly, tumor cells are propagated in vitro in cell culture. Prior to injection into the animals, the cell lines are washed and suspended in buffer, at a cell density of about 10×106 to 10×107 cells/ml. The animals are then infected subcutaneously with 10 to 100 μl of the cell suspension, allowing one to three weeks for a tumor to appear.

In addition, the Lewis lung (3LL) carcinoma of mice, which is one of the most thoroughly studied experimental tumors, can be used as an investigational tumor model. Efficacy in this tumor model has been correlated with beneficial effects in the treatment of human patients diagnosed with small-cell carcinoma of the lung (SCCL). This tumor can be introduced in normal mice upon injection of tumor fragments from an affected mouse or of cells maintained in culture. Zupi et al., Br. J. Cancer, 41: suppl. 4, 30 (1980). Evidence indicates that tumors can be started from injection of even a single cell and that a very high proportion of infected tumor cells survive. For further information about this tumor model see, Zacharski, Haemostasis, 16: 300-320 (1986).

One way of evaluating the efficacy of a test compound in an animal model with an implanted tumor is to measure the size of the tumor before and after treatment. Traditionally, the size of implanted tumors has been measured with a slide caliper in two or three dimensions. The measure limited to two dimensions does not accurately reflect the size of the tumor; therefore, it is usually converted into the corresponding volume by using a mathematical formula. However, the measurement of tumor size is very inaccurate. The therapeutic effects of a drug candidate can be better described as treatment-induced growth delay and specific growth delay. Another important variable in the description of tumor growth is the tumor volume doubling time. Computer programs for the calculation and description of tumor growth are also available, such as the program reported by Rygaard and Spang-Thomsen, Proc. 6th Int. Workshop on Immune-Deficient Animals, Wu and Sheng eds. (Basel, 1989), p. 301. It is noted, however, that necrosis and inflammatory responses following treatment may actually result in an increase in tumor size, at least initially. Therefore, these changes need to be carefully monitored, by a combination of a morphometric method and flow cytometric analysis.

Further, recombinant (transgenic) animal models can be engineered by introducing the coding portion of the PA genes identified herein into the genome of animals of interest, using standard techniques for producing transgenic animals. Animals that can serve as a target for transgenic manipulation include, without limitation, mice, rats, rabbits, guinea pigs, sheep, goats, pigs, and non-human primates, e.g., baboons, chimpanzees and monkeys. Techniques known in the art to introduce a transgene into such animals include pronucleic microinjection (U.S. Pat. No. 4,873,191); retrovirus-mediated gene transfer into germ lines (e.g., Van der Putten et al., Proc. Natl. Acad. Sci. USA, 82: 6148-615 (1985)); gene targeting in embryonic stem cells (Thompson et al., Cell, 56: 313-321 (1989)); electroporation of embryos (Lo, Mol. Cell. Biol., 3: 1803-1814 (1983)); and sperm-mediated gene transfer. Lavitrano et al., Cell, 57: 717-73 (1989). For a review, see for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA, 89: 6232-636 (1992). The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry. The animals are further examined for signs of tumor or cancer development.

Alternatively, “knock-out” animals can be constructed that have a defective or altered gene encoding a PA polypeptide identified herein, as a result of homologous recombination between the endogenous gene encoding the PA polypeptide and altered genomic DNA encoding the same polypeptide introduced into an embryonic cell of the animal. For example, cDNA encoding a particular PA polypeptide can be used to clone genomic DNA encoding that polypeptide in accordance with established techniques. A portion of the genomic DNA encoding a particular PA polypeptide can be deleted or replaced with another gene, such as a gene encoding a selectable marker that can be used to monitor integration. Typically, several kilobases of unaltered flanking DNA (both at the 5′ and 3′ ends) are included in the vector. See, e.g., Thomas and Capecchi, Cell, 51: 503 (1987) for a description of homologous recombination vectors. The vector is introduced into an embryonic stem cell line (e.g., by electroporation) and cells in which the introduced DNA has homologously recombined with the endogenous DNA are selected. See, e.g., Li et al., Cell, 69: 915 (1992). The selected cells are then injected into a blastocyst of an animal (e.g., a mouse or rat) to form aggregation chimeras. See, e.g., Bradley, in Teratocarcinomas and Embryonic Stem Cells: A Practical Approach, E. J. Robertson, ed. (IRL: Oxford, 1987), pp. 113-152. A chimeric embryo can then be implanted into a suitable pseudopregnant female foster animal and the embryo brought to term to create a “knock-out” animal. Progeny harboring the homologously recombined DNA in their germ cells can be identified by standard techniques and used to breed animals in which all cells of the animal contain the homologously recombined DNA. Knock-out animals can also be generated, as is well knows in the art, by administering an antisense molecule of the invention. Animals comprising such antisense molecules are specifically contemplanted as an embodiment of the invention. Knockout animals can be characterized, for instance, by their ability to defend against certain pathological conditions and by their development of pathological conditions due to absence (knock-out) of the PA polypeptides.

The efficacy of antibodies specifically binding the PA polypeptides identified herein, and other drug candidates, can be tested also in the treatment of spontaneous animal tumors. A suitable target for such studies is the feline oral squamous cell carcinoma (SCC). Feline oral SCC is a highly invasive, malignant tumor that is the most common oral malignancy of cats, accounting for over 60% of the oral tumors reported in this species. It rarely metastasizes to distant sites, although this low incidence of metastasis may merely be a reflection of the short survival times for cats with this tumor. These tumors are usually not amenable to surgery, primarily because of the anatomy of the feline oral cavity. At present, there is no effective treatment for this tumor. Prior to entry into the study, each cat undergoes complete clinical examination and biopsy, and is scanned by computed tomography (CT). Cats diagnosed with sublingual oral squamous cell tumors are excluded from the study. The tongue can become paralyzed as a result of such tumor, and even if the treatment kills the tumor, the animals may not be able to feed themselves. Each cat is treated repeatedly, over a longer period of time. Photographs of the tumors will be taken daily during the treatment period, and at each subsequent recheck. After treatment, each cat undergoes another CT scan. CT scans and thoracic radiograms are evaluated every 8 weeks thereafter. The data are evaluated for differences in survival, response, and toxicity as compared to control groups. Positive response may require evidence of tumor regression, preferably with improvement of quality of life and/or increased life span.

In addition, other spontaneous animal tumors, such as fibrosarcoma, adenocarcinoma, lymphoma, chondroma, or leiomyosarcoma of dogs, cats, and baboons can also be tested. Of these, mammary adenocarcinoma in dogs and cats is a preferred model as its appearance and behavior are very similar to those in humans. However, the use of this model is limited by the rare occurrence of this type of tumor in animals.

Other in vitro and in vivo angiogenic tests known in the art are also suitable herein.

Consequently, the PA polypeptides described herein can also be employed as therapeutic agents. The PA polypeptides of the present invention can be formulated according to known methods to prepare pharmaceutically useful compositions, whereby the PA product hereof is combined in admixture with a pharmaceutically acceptable carrier vehicle. Therapeutic formulations are prepared for storage by mixing the active ingredient having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients or stabilizers are nontoxic to recipients at the dosages and concentrations employed, and include buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagine, arginine or lysine; monosaccharides, disaccharides and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

The formulations to be used for in vivo administration must be sterile. This is readily accomplished by filtration through sterile filtration membranes, prior to or following lyophilization and reconstitution.

Therapeutic compositions herein generally are placed into a container having a sterile access port, for example, an intravenous solution bag or vial having a stopper pierceable by a hypodermic injection needle.

The route of administration is in accord with known methods, e.g. injection or infusion by intravenous, intraperitoneal, intracerebral, intramuscular, intraocular, intraarterial or intralesional routes, topical administration, or by sustained release systems.

Dosages and desired drug concentrations of pharmaceutical compositions of the present invention can vary depending on the particular use envisioned. The determination of the appropriate dosage or route of administration is well within the skill of an ordinary skilled artisan. Animal experiments provide reliable guidance for the determination of effective doses for human therapy. Interspecies scaling of effective doses can be performed following the principles laid down by Mordenti, J. and Chappell, W. “The use of interspecies scaling in toxicokinetics” In Toxicokinetics and New Drug Development, Yacobi et al., Eds., Pergamon Press, New York 1989, pp. 42-96.

When in vivo administration of a PA polypeptide or agonist or antagonist thereof is employed, normal dosage amounts can vary from about 10 ng/kg to up to 100 mg/kg of mammal body weight or more per day, preferably about 1 μg/kg/day to 10 mg/kg/day, depending upon the route of administration. Guidance as to particular dosages and methods of delivery is provided in the literature; see, for example, U.S. Pat. Nos. 4,657,760; 5,206,344; or 5,225,212. It is anticipated that different formulations will be effective for different treatment compounds and different disorders, that administration targeting one organ or tissue, for example, can necessitate delivery in a manner different from that of another organ or tissue.

Where sustained-release administration of a PA polypeptide is desired in a formulation with release characteristics suitable for the treatment of any disease or disorder requiring administration of the PA polypeptide, microencapsulation of the PA polypeptide is contemplated. Microencapsulation of recombinant proteins for sustained release has been successfully performed with human growth hormone (rhGH), interferon-(rhIFN-), interleukin-2, and MN rgp120. Johnson et al., Nat. Med., 2:795-799 (1996); Yasuda, Biomed. Ther., 27:1221-1223 (1993); Hora et al., Bio/Technology, 8:755-758 (1990); Cleland, “Design and Production of Single Immunization Vaccines Using Polylactide Polyglycolide Microsphere Systems,” in Vaccine Design: The Subunit and Adjuvant Approach, Powell and Newman, eds, (Plenum Press: New York, 1995), pp. 439-462; WO 97/03692, WO 96/40072, WO 96/07399; and U.S. Pat. No. 5,654,010.

The sustained-release formulations of these proteins were developed using poly-lactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Lewis, “Controlled release of bioactive agents from lactide/glycolide polymer,” in: M. Chasin and R. Langer (Eds.), Biodegradable Polymers as Drug Delivery Systems (Marcel Dekker: New York, 1990), pp. 1-41.

This invention encompasses methods of screening compounds to identify those that mimic the PA polypeptide (agonists) or prevent the effect of the PA polypeptide (antagonists). Screening assays for antagonist drug candidates are designed to identify compounds that bind or complex with the PA polypeptides encoded by the genes identified herein, or otherwise interfere with the interaction of the encoded polypeptides with other cellular proteins. Such assays include methods identifying compounds that interfere with the interaction of a gene (mRNA or genomic DNA) encoding a PA polypeptide, such as those described herein. These screening assays will include assays amenable to high- or ultra-high-throughput screening of chemical libraries, making them particularly suitable for identifying antisense and small molecule drug candidates.

The assays can be performed in a variety of formats, including protein-protein binding assays, biochemical screening assays, immunoassays, target nucleic acid binding assays, and cell-based assays, which are well characterized in the art.

In certain embodiments, assays for antagonists entail contacting the drug candidate with a PA polypeptide encoded by a nucleic acid identified herein under conditions and for a time sufficient to allow these two components to interact.

In binding assays, the interaction is binding and the complex formed can be isolated or detected in the reaction mixture. In a particular embodiment, the PA polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the PA polypeptide and drying. Alternatively, an immobilized antibody, e.g., a monoclonal antibody, specific for the PA polypeptide to be immobilized can be used to anchor it to a solid surface. The assay is performed by adding the non-immobilized component, which can be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular PA polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a PA polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo can be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner. A particularly useful assay system is a microarray assay, such as chip upon which a nucleic acid fragment-sequence library—based on the PA gene sequence—is synthesized.

Oligonucleotides or longer fragments derived from any of the polynucleotide sequences described herein can be used as targets in a microarray. The microarray can be used to monitor the expression level of large numbers of genes simultaneously (to produce a transcript image), to identify genetic variants, mutations and polymorphisms, to identify effective nucleic acid binding molecules such as antisense molecules, regulatory proteins, ribosomes or polymerases. This information may be used to determine gene function, to understand the genetic basis of disease, to diagnose disease, to identify therapeutic molecules (e.g., antisense), and to develop, and monitor the activities of therapeutic agents.

In one embodiment, the microarray can be prepared and used according to the methods known in the art, such as those described in WO95/11995 (Chee et al.), Lockhart, D. J., et al. (Nat. Biotech. 14: 1675-1680 (1996)), and Schena, M., et al. (Proc. Natl. Acad. Sci. 93: 10614-10619 (1996)) or in WO 99/24463.

The microarray is preferably composed of a large number of unique, single-stranded nucleic acid sequences, usually either synthetic antisense oligonucleotides or fragments of cDNAs, fixed to a solid support. The oligonucleotides are preferably about 6-60 nucleotides in length, more preferably about 15 to 30 nucleotides in length, and most preferably about 20 to 25 nucleotides in length. For a certain type of microarray, it may be preferable to use oligonucleotides that are only 7 to 10 nucleotides in length. The microarray can contain oligonucleotides which cover the known 5′ (or 3′) sequence or untranslated regions, sequential oligonucleotides which cover the full-length sequence or unique oligonucleotides selected from particular areas along the length of the sequence including untranslated regions. Polynucleotides used in the microarray can be oligonucleotides that are specific to a gene or genes of interest, preferably a PA gene, in which at least a fragment of the sequence is known or that are specific to one or more unidentified cDNAs that are common to a particular cell or tissue type or to a normal, developmental, or disease state. In certain situations, it is appropriate to use pairs of oligonucleotides on a microarray. The pairs will be identical, except for one nucleotide preferably located in the center of the sequence. The second oligonucleotide in the pair (mismatched by one) serves as a control. The number of oligonucleotide pairs may range from 2 to 1,000,000. Microarrays can also contain fragments in DNA duplex form, which are particularly useful in identifying molecules that bind to PA genomic DNA.

For producing oligonucleotides to a known sequence for a microarray, the gene of interest is examined using a computer algorithm which starts at the 5′ or more preferably at the 3′ end of the nucleotide sequence. The algorithm identifies oligomers of defined length that are unique to the gene, have a GC content within a range suitable for hybridization, and lack predicted secondary structure that may interfere with hybridization.

In one aspect, the oligonucleotides are synthesized at designated areas on the surface of a substrate, for example by using a light-directed chemical coupling procedure and an inkjet application apparatus, such as that described in WO95/251116 (Baldeschweiler et al.). The substrate may be paper, nylon or any other type of membrane, filter, chip, glass slide, or any other suitable solid support. In another aspect, a “gridded” array analogous to a dot or slot blot (HYBRIDOT apparatus, GIBCO/BRL) may be used to arrange and link cDNA fragments or oligonucleotides to the surface of a substrate using a vacuum system, thermal, UV, mechanical or chemical bonding procedures. In yet another aspect, an array may be produced by hand or by using available devices, materials, and machines (including BRINKMANN multichannel pipettors or robotic instruments). Such an array may contain 8, 24, 96, 384, 1536, or 6144 oligonucleotides, or any other multiple from 2 to 1,000,000 that lends itself to the efficient use of commercially available instrumentation.

Sample analysis using the microarrays can be conducted by extracting polynucleotides from a biological sample. The biological samples are obtained from any bodily fluid (blood, urine, saliva, phlegm, gastric juices, etc.), cultured cells, biopsies, or other tissue preparations. The polynucleotides extracted from the sample can be used to produce, as probes, nucleic acid sequences that are complementary to the nucleic acids on the microarray. If the microarray consists of cDNAs, antisense RNAs (aRNA) are appropriate probes. Therefore, in one aspect, mRNA is used to produce cDNA that, in turn and in the presence of fluorescent nucleotides, is used to produce fragment or oligonucleotide aRNA probes. These fluorescently-labeled probes are incubated with the microarray so that the probe sequences hybridize to the cDNA oligonucleotides of the microarray. In another aspect, nucleic acid sequences used as probes can include polynucleotides, fragments, and complementary or antisense sequences produced using restriction enzymes, PCR technologies, and OLIGOLABELING™ or TRANSPROBE™ kits (Pharmacia) well known in the area of hybridization technology. In an alternative microarray embodiment, oligonucleotides (preferably antisense molecules) are employed on the support and the target cDNA is the soluble binding component of the assay.

Incubation conditions are adjusted so that hybridization occurs with precise complementary matches or with various degrees of less complementarity. After removal of nonhybridized probes, a scanner is used to determine the levels and patterns of fluorescence. The scanned images are examined to determine degree of complementarity and the relative abundance of each oligonucleotide sequence on the microarray. A detection system may be used to measure the absence, presence, and amount of hybridization for all of the distinct sequences simultaneously. This data may be used for large-scale correlation studies or functional analysis of the sequences, mutations, variants, or polymorphisms among samples (Heller, R. A., et al., Proc. Natl. Acad. Sci. 94: 2150-55 (1997)).

For gene mapping, a gene or a cloned DNA fragment is hybridized to an ordered array of DNA fragments, and the identity of the DNA elements applied to the array is unambiguously established by the pixel or pattern of pixels of the array that are detected. In constructing physical maps of the genome, arrays of immobilized cloned DNA fragments are hybridized with other cloned DNA fragments to establish whether the cloned fragments in the probe mixture overlap and are therefore contiguous to the immobilized clones on the array. For example, Meier-Ewert et al., (J. Biotech. 35(2-3):191-203 (1994)) disclose such an application.

The arrays of immobilized DNA fragments may also be used for genetic diagnostics. For example, array containing multiple forms of a mutated gene or genes can be probed with a labeled mixture of a patient's DNA which will preferentially interact with only one of the immobilized versions of the gene. The detection of this interaction can provide a medical diagnosis. Arrays of immobilized DNA fragments can also be used in DNA probe diagnostics. For unambiguous genotyping or identifying a DNA- or RNA-containing sample as that of a human, the identity of the test sample can be established unambiguously by hybridizing the sample to an array containing DNA from different organisms, including human, wherein one or more PA genes sequences are included in the array. Other molecules of genetic interest, such as cDNAs and RNAs can be immobilized on the array or alternately used as the labeled probe mixture that is applied to the array.

In one embodiment, to assay for antagonists, the PA polypeptide can be added to a cell or expressed in a host cell, the cell contacted with the antagonist compound, and the ability of the compound to inhibit the activity of interest in the presence of the PA polypeptide indicates that the compound is an antagonist to the PA polypeptide. Alternatively, antagonists can be detected by combining the PA polypeptide and a potential antagonist with membrane-bound PA polypeptide receptors or recombinant receptors or a PA binding protein under appropriate conditions for a competitive inhibition assay. The PA polypeptide can be labeled, such as by radioactivity, such that the number of PA polypeptide molecules bound to the receptor or binding protein can be used to determine the effectiveness of the potential antagonist. The gene encoding the receptor or binding protein can be identified by numerous methods known to those of skill in the art, for example, ligand panning and FACS sorting (Coligan et al., Current Protocols in Immun., 1(2): Chapter 5 (1991)).

Preferably, expression cloning is employed wherein polyadenylated RNA is prepared from a cell responsive to the PA polypeptide and a cDNA library created from this RNA is divided into pools and used to transfect COS cells or other cells that are not responsive to or do not contain binding protein activity to the PA polypeptide. Transfected cells that are grown on glass slides are exposed to labeled PA polypeptide or lysates are prepared for testing binding activity. The PA polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor or binding protein. As an alternative approach for receptor or binding protein identification, labeled PA polypeptide can be photoaffinity-linked with cell membrane or extract preparations that express or contain the receptor or binding protein. Cross-linked material is resolved by PAGE and exposed to X-ray film. The labeled complex can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor or binding protein.

More specific examples of potential antagonists include a polypeptide that binds to the fusions of immunoglobulin with PA polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist can be a closely related protein, for example, a mutated form of the PA polypeptide that recognizes the receptor or binding protein but imparts no effect, thereby competitively inhibiting the action of the PA polypeptide.

Another potential PA polypeptide antagonist is an antisense construct prepared using antisense technology, where, for example, the antisense molecule acts to block directly the translation of mRNA (or transcription) by hybridizing to targeted mRNA (or genomic DNA) and preventing protein translation (or mRNA transcription) of PA. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature PA polypeptides herein, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al., Science, 241: 456 (1988); Dervan et al., Science, 251:1360 (1991)), thereby preventing transcription and the production of the PA polypeptide. The antisense RNA oligonucleotide hybridizes to the mRNA in vivo and blocks translation of the mRNA molecule into the PA polypeptide (antisense—Okano, Neurochem., 56:560 (1991); Olivodeoxynucleotides as Antisense Inhibitors of Gene Expression (CRC Press: Boca Raton, Fla., 1988). The oligonucleotides described above can also be delivered to cells such that the antisense RNA or DNA can be expressed in vivo to inhibit production of the PA polypeptide. When antisense DNA is used, oligodeoxyribonucleotides derived from the translation-initiation site, e.g., between about −10 and +10 positions of the target gene nucleotide sequence, are preferred.

Potential antagonists include small molecules that bind to the active site, the protein binding site, or other relevant binding site (e.g., co-factor binding site, substrate binding site) of the PA polypeptide, thereby blocking the normal biological activity of the PA polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra. Such molecules can have backbone bonds not naturally found in DNA or RNA.

These small molecules can be identified by any one or more of the screening assays discussed herein and/or by any other screening techniques well known for those skilled in the art.

### Anti-PA Antibodies

The present invention further provides anti-PA polypeptide antibodies. Exemplary antibodies include polyclonal, monoclonal, humanized, bispecific, and heteroconjugate antibodies.

Polyclonal Antibodies

The anti-PA polypeptide antibodies can comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent can include the PA polypeptide or a fusion protein thereof. It can be useful to conjugate the immunizing agent to a protein known to be immunogenic in the mammal being immunized. Examples of such immunogenic proteins include but are not limited to keyhole limpet hemocyanin, serum albumin, bovine thyroglobulin, and soybean trypsin inhibitor. Examples of adjuvants which can be employed include Freund's complete adjuvant and MPL-TDM adjuvant (monophosphoryl Lipid A, synthetic trehalose dicorynomycolate). The immunization protocol can be selected by one skilled in the art without undue experimentation.

Monoclonal Antibodies

The anti-PA antibodies can, alternatively, be monoclonal antibodies. Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the PA polypeptide or a fusion protein thereof Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against PA. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be subcloned by limiting dilution procedures and grown by standard methods (Goding, supra). Suitable culture media for this purpose include, for example, Dulbecco's Modified Eagle's Medium and RPMI-1640 medium. Alternatively, the hybridoma cells can be grown in vivo as ascites in a mammal. The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies can be monovalent antibodies. Methods for preparing monovalent antibodies are well known in the art. For example, one method involves recombinant expression of immunoglobulin light chain and modified heavy chain. The heavy chain is truncated generally at any point in the Fc region so as to prevent heavy chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly, Fab fragments, can be accomplished using routine techniques known in the art.

Human and Humanized Antibodies

The anti-PA antibodies of the invention can further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-329 (1988); and Presta, Curr. Op. Struct. Biol., 2:593-596 (1992)).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries (Hoogenboom and Winter, J. Mol. Biol., 227:381 (1991); Marks et al., J. Mol. Biol., 222:581 (1991)). The techniques of Cole et al. and Boemer et al. are also available for the preparation of human monoclonal antibodies (Cole et. al., Monoclonal Antibodies and Cancer Therapy, Alan R. Liss, p. 77 (1985) and Boemer et al., J. Immunol., 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10, 779-783 (1992); Lonberg et al., Nature 368 856-859 (1994); Morrison, Nature 368, 812-13 (1994); Fishwild et al., Nature Biotechnology 14, 845-51 (1996); Neuberger, Nature Biotechnology 14, 826 (1996); Lonberg and Huszar, Intern. Rev. Immunol. 13 65-93 (1995).

Bispecific Antibodies

Bispecific antibodies are monoclonal, preferably human or humanized, antibodies that have binding specificities for at least two different antigens. In the present case, one of the binding specificities is for the PA, the other one is for any other antigen, and preferably for a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers, which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various technique for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60(1991).

Exemplary bispecific antibodies can bind to two different epitopes on a given PA polypeptide herein. Alternatively, an anti-PA polypeptide arm can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular PA polypeptide. Bispecific antibodies can also be used to localize cytotoxic agents to cells, which express a particular PA polypeptide. These antibodies possess a PA-binding arm and an arm, which binds a cytotoxic agent or a radionuclide chelator, such as EOTUBE, DPTA, DOTA, or TETA. Another bispecific antibody of interest binds the PA polypeptide and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconiugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PA polypeptideI, PA polypeptideII, and PA polypeptide-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl)hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Pharmaceutical Compositions of Antibodies

Antibodies specifically binding a PA polypeptide identified herein, as well as other molecules identified by the screening assays disclosed herein, can be administered for the treatment of various disorders in the form of pharmaceutical compositions.

If the PA polypeptide is intracellular and whole antibodies are used as inhibitors, internalizing antibodies are preferred. However, lipofections or liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules.

Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37 C, resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhlydryl residues, lyophilizing from acidic solutions, controlling moisture content, using appropriate additives, and developing specific polymer matrix compositions.

### Uses for Anti-PA Antibodies

The anti-PA antibodies of the invention have various utilities. For example, anti-PA antibodies can be used in diagnostic assays for a PA, e.g., detecting its expression in specific cells, tissues, or serum. Various diagnostic assay techniques known in the art can be used, such as competitive binding assays, direct or indirect sandwich assays and immunoprecipitation assays conducted in either heterogeneous or homogeneous phases (Zola, Monoclonal Antibodies: A Manual of Techniques, CRC Press, Inc. (1987) pp. 147-158). The antibodies used in the diagnostic assays can be labeled with a detectable moiety. The detectable moiety should be capable of producing, either directly or indirectly, a detectable signal. For example, the detectable moiety can be a radioisotope, such as ³H, ¹⁴C, ³²P, ³⁵S, or ¹²⁵I, a fluorescent or chemiluminescent compound, such as fluorescein isothiocyanate, rhodamine, or luciferin, or an 30 enzyme, such as alkaline phosphatase, beta-galactosidase or horseradish peroxidase. Any method known in the art for conjugating the antibody to the detectable moiety can be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982).

Anti-PA polypeptide antibodies also are useful for the affinity purification of PA from recombinant cell culture or natural sources. In this process, the antibodies against PA are immobilized on a suitable support, such a Sephadex resin or filter paper, using methods well known in the art. The immobilized antibody then is contacted with a sample containing the PA to be purified, and thereafter the support is washed with a suitable solvent that will remove substantially all the material in the sample except the PA, which is bound to the immobilized antibody. Finally, the support is washed with another suitable solvent that will release the PA from the antibody.

### Nucleic Acid Diagnostic Assays

This invention is also related to the use of the nucleic acid sequence encoding the PA polypeptide as a diagnostic. Detection of a mutated form of the PA polypeptide will allow a diagnosis of an angiogenic disease or a susceptibility to an angiogenic disease, such as a tumor, since mutations in the PA polypeptide may cause tumors.

Individuals carrying mutations in the genes encoding a human PA polypeptide may be detected at the DNA level by a variety of techniques. Nucleic acids for diagnosis may be obtained from a patient's cells, such as from blood, urine, saliva, tissue biopsy, and autopsy material. The genomic DNA may be used directly for detection or may be amplified enzymatically by using PCR (Saiki et al., Nature, 324: 163-166 (1986)) prior to analysis. RNA or cDNA may also be used for the same purpose. As an example, PCR primers complementary to the nucleic acid encoding the PA polypeptide can be used to identify and analyze PA polypeptide mutations. For example, deletions and insertions can be detected by a change in size of the amplified product in comparison to the normal genotype. Point mutations can be identified by hybridizing amplified DNA to radiolabeled RNA encoding the PA polypeptide, or alternatively, radiolabeled antisense DNA sequences encoding the PA polypeptide. Perfectly matched sequences can be distinguished from mismatched duplexes by RNase A digestion or by differences in melting temperatures.

Genetic testing based on DNA sequence differences may be achieved by detection of alteration in electrophoretic mobility of DNA fragments in gels with or without denaturing agents. Small sequence deletions and insertions can be visualized by high resolution gel electrophoresis. DNA fragments of different sequences may be distinguished on denaturing formamidine gradient gels in which the mobilities of different DNA fragments are retarded in the gel at different positions according to their specific melting or partial melting temperatures. See, e.g., Myers et al., Science, 230: 1242 (1985).

Sequence changes at specific locations may also be revealed by nuclease protection assays, such as RNase and SI protection or the chemical cleavage method, for example, Cotton etal., Proc. Natl. Acad. Sci. USA, 85: 4397-4401 (1985).

Thus, the detection of a specific DNA sequence may be achieved by methods such as hybridization, RNase protection, chemical cleavage, direct DNA sequencing, or the use of restriction enzymes, e.g., restriction fragment length polymorphisms (RFLP), and Southern blotting of genomic DNA.

### Detecting Polypeptide Levels

In addition to more conventional gel-electrophoresis and DNA sequencing, mutations can also be detected by in situ analysis.

Expression of nucleic acid encoding the PA polypeptide may be linked to vascular disease or neovascularization associated with tumor formation. If the PA polypeptide has a signal sequence and the mRNA is highly expressed in endothelial cells and to a lesser extent in smooth muscle cells, this indicates that the PA polypeptide is present in serum. Accordingly, an anti-PA polypeptide antibody could be used to diagnose vascular disease or neovascularization associated with tumor formation, since an altered level of this PA polypeptide may be indicative of such disorders.

A competition assay may be employed wherein antibodies specific to the PA polypeptide are attached to a solid support and the labeled PA polypeptide and a sample derived from the host are passed over the solid support and the amount of label detected attached to the solid support can be correlated to a quantity of PA polypeptide in the sample.

### Screening Assays for Drug Candidates

This invention also encompasses methods of screening compounds to identify those that mimic the PA polypeptide (agonists) or prevent the effect of the PA polypeptide (antagonists), i.e., those which promote or inhibit, respectively, angiogenesis in vivo or tube formation of endothelial cells in vitro. Screening assays for antagonist drug candidates are designed to identify compounds that bind or complex with the PA polypeptide encoded by the genes identified herein, or otherwise interfere with the interaction of the encoded polypeptides with other cellular proteins. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays, which are well characterized in the art.

All assays for antagonists are common in that they call for contacting the drug candidate with a PA polypeptide encoded by a nucleic acid identified herein under conditions and for a time sufficient to allow these two components to interact.

In binding assays, the interaction is binding and the complex formed can be isolated or detected in the reaction mixture. In a particular embodiment, the PA polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the PA polypeptide and drying. Alternatively, an immobilized antibody, e.g., a monoclonal antibody, specific for the PA polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular PA polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340: 245-246 (1989); Chien et al., Proc. Natl. Acad. Sci. USA, 88: 9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a PA polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

If the PA polypeptide has the ability to stimulate the proliferation of endothelial cells in the presence of the co-mitogen ConA, then one example of a screening method takes advantage of this ability. Specifically, in the proliferation assay, human umbilical vein endothelial cells are obtained and cultured in 96-well flat-bottomed culture plates (Costar, Cambridge, Mass.) and supplemented with a reaction mixture appropriate for facilitating proliferation of the cells, the mixture containing Con-A (Calbiochem, La Jolla, Calif.). Con-A and the compound to be screened are added and after incubation at 37 C, cultures are pulsed with 3-H-thymidine and harvested onto glass fiber filters (phD; Cambridge Technology, Watertown, Mass.). Mean 3-H-thymidine incorporation (cpm) of triplicate cultures is determined using a liquid scintillation counter (Beckman Instruments, Irvine, Calif.). Significant 3-(H)thymidine incorporation indicates stimulation of endothelial cell proliferation.

To assay for antagonists, the assay described above is performed; however, in this assay the PA polypeptide is added along with the compound to be screened and the ability of the compound to inhibit 3-(H)thymidine incorporation in the presence of the PA polypeptide indicates that the compound is an antagonist to the PA polypeptide. Alternatively, antagonists may be detected by combining the PA polypeptide and a potential antagonist with membrane-bound PA polypeptide receptors or recombinant receptors under appropriate conditions for a competitive inhibition assay. The PA polypeptide can be labeled, such as by radioactivity, such that the number of PA polypeptide molecules bound to the receptor can be used to determine the effectiveness of the potential antagonist. The gene encoding the receptor can be identified by numerous methods known to those of skill in the art, for example, ligand panning and FACS sorting. Coligan et al., Current Protocols in Immun., 1(2): Chapter 5 (1991). Preferably, expression cloning is employed wherein polyadenylated RNA is prepared from a cell responsive to the PA polypeptide and a cDNA library created from this RNA is divided into pools and used to transfect COS cells or other cells that are not responsive to the PA polypeptide. Transfected cells that are grown on glass slides are exposed to the labeled PA polypeptide. The PA polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, labeled PA polypeptide can be photoaffinity-linked with cell membrane or extract preparations that express the receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

In another assay for antagonists, mammalian cells or a membrane preparation expressing the receptor would be incubated with labeled PA polypeptide in the presence of the candidate compound. The ability of the compound to enhance or block this interaction could then be measured.

The compositions useful in the treatment of angiogenic disorders include, without limitation, antibodies, small organic and inorganic molecules, peptides, phosphopeptides, antisense and ribozyme molecules, triple-helix molecules, etc., that inhibit the expression and/or activity of the target gene product.

More specific examples of potential antagonists include an oligonucleotide that binds to the fusions of immunoglobulin with a PA polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the PA polypeptide that recognizes the receptor but imparts no effect, thereby competitively inhibiting the action of the PA polypeptide.

Another potential PA polypeptide antagonist or agonist is an antisense RNA or DNA construct prepared using antisense technology, where, e.g., an antisense RNA or DNA molecule acts to block directly the translation of mRNA by hybridizing to targeted mRNA and preventing protein translation. Antisense technology can be used to control gene expression through triple-helix formation or antisense DNA or RNA, both of which methods are based on binding of a polynucleotide to DNA or RNA. For example, the 5′ coding portion of the polynucleotide sequence, which encodes the mature PA polypeptides herein, is used to design an antisense RNA oligonucleotide of from about 10 to 40 base pairs in length. A DNA oligonucleotide is designed to be complementary to a region of the gene involved in transcription (triple helix—see, Lee et al., Nucl. Acids Res., 6:3073 (1979); Cooney et al., Science, 241: 456 (1988); Dervan et al., Science, 251:1360 (1991)), thereby preventing transcription and the production of the PA polypeptide. The antisense RNA oligonucleotide hybridizes to the mRNA in vivo and blocks translation of the mRNA molecule into the PA polypeptide (antisense—Okano, Neurochem., 56:560 (1991); Oligodeoxynucleotides as Antisense Inhibitors of Gene Expression (CRC Press: Boca Raton, Fla., 1988). The oligonucleotides described above can also be delivered to cells such that the antisense RNA or DNA may be expressed in vivo to inhibit production of the PA polypeptide. When antisense DNA is used, oligodeoxyribonucleotides derived from the translation-initiation site, e.g., between about −10 and +10 positions of the target gene nucleotide sequence, are preferred.

Antisense RNA or DNA molecules are generally at least about 5 bases in length, about 10 bases in length, about 15 bases in length, about 20 bases in length, about 25 bases in length, about 30 bases in length, about 35 bases in length, about 40 bases in length, about 45 bases in length, about 50 bases in length, about 55 bases in length, about 60 bases in length, about 65 bases in length, about 70 bases in length, about 75 bases in length, about 80 bases in length, about 85 bases in length, about 90 bases in length, about 95 bases in length, about 100 bases in length, or more.

Potential antagonists include small molecules that bind to the active site, the receptor binding site, or growth factor or other relevant binding site of the PA polypeptide, thereby blocking the normal biological activity of the PA polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4: 469-471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

### Drug Screening

This invention is particularly useful for screening compounds by using PA polypeptides or binding fragment thereof in any of a variety of drug screening techniques. The PA polypeptide or fragment employed in such a test can either be free in solution, affixed to a solid support, borne on a cell surface, or located intracellularly. One method of drug screening utilizes eukaryotic or prokaryotic host cells which are stably transformed with recombinant nucleic acids expressing the PA polypeptide or fragment. Drugs are screened against such transformed cells in competitive binding assays. Such cells, either in viable or fixed form, can be used for standard binding assays. One can measure, for example, the formation of complexes between PA polypeptide or a fragment and the agent being tested. Alternatively, one can examine the diminution in complex formation between the PA polypeptide and its target cell or target receptors caused by the agent being tested.

Thus, the present invention provides methods of screening for drugs or any other agents which can affect a PA polypeptide-associated disease or disorder. These methods comprise contacting such an agent with an PA polypeptide or fragment thereof and assaying (I) for the presence of a complex between the agent and the PA polypeptide or fragment, or (ii) for the presence of a complex between the PA polypeptide or fragment and the cell, by methods well known in the art. In such competitive binding assays, the PA polypeptide or fragment is typically labeled. After suitable incubation, free PA polypeptide or fragment is separated from that present in bound form, and the amount of free or uncomplexed label is a measure of the ability of the particular agent to bind to PA polypeptide or to interfere with the PA polypeptide/cell complex.

Another technique for drug screening provides high throughput screening for compounds having suitable binding affinity to a polypeptide and is described in detail in WO 84/03564, published on Sep. 13, 1984. Briefly stated, large numbers of different small peptide test compounds are synthesized on a solid substrate, such as plastic pins or some other surface. As applied to a PA polypeptide, the peptide test compounds are reacted with PA polypeptide and washed. Bound PA polypeptide is detected by methods well known in the art. Purified PA polypeptide can also be coated directly onto plates for use in the aforementioned drug screening techniques. In addition, non-neutralizing antibodies can be used to capture the peptide and immobilize it on the solid support.

This invention also contemplates the use of competitive drug screening assays in which neutralizing antibodies capable of binding PA polypeptide specifically compete with a test compound for binding to PA polypeptide or fragments thereof. In this manner, the antibodies can be used to detect the presence of any peptide which shares one or more antigenic determinants with PA polypeptide.

### Rational Drug Design

The goal of rational drug design is to produce structural analogs of biologically active polypeptide of interest (i.e., a PA polypeptide) or of small molecules with which they interact, e.g., agonists, antagonists, or inhibitors. Any of these examples can be used to fashion drugs which are more active or stable forms of the PA polypeptide or which enhance or interfere with the function of the PA polypeptide in vivo (c.f., Hodgson, Bio/Technology, 9: 19-21 (1991)).

In one approach, the three-dimensional structure of the PA polypeptide, or of a PA polypeptide-inhibitor complex, is determined by x-ray crystallography, by computer modeling or, most typically, by a combination of the two approaches. Both the shape and charges of the PA polypeptide must be ascertained to elucidate the structure and to determine active site(s) of the molecule. Less often, useful information regarding the structure of the PA polypeptide can be gained by modeling based on the structure of homologous proteins. In both cases, relevant structural information is used to design analogous PA polypeptide-like molecules or to identify efficient inhibitors. Useful examples of rational drug design can include molecules which have improved activity or stability as shown by Braxton and Wells, Biochemistry, 31:7796-7801 (1992) or which act as inhibitors, agonists, or antagonists of native peptides as shown by Athauda et al, J. Biochem., 113:742-746 (1993).

It is also possible to isolate a target-specific antibody, selected by functional assay, as described above, and then to solve its crystal structure. This approach, in principle, yields a pharmacore upon which subsequent drug design can be based. It is possible to bypass protein crystallography altogether by generating anti-idiotypic antibodies (anti-ids) to a functional, pharmacologically active antibody. As a mirror image of a mirror image, the binding site of the anti-ids would be expected to be an analog of the original receptor. The anti-id could then be used to identify and isolate peptides from banks of chemically or biologically produced peptides. The isolated peptides would then act as the pharmacore.

By virtue of the present invention, sufficient amounts of the PA polypeptide can be made available to perform such analytical studies as X-ray crystallography. In addition, knowledge of the PA polypeptide amino acid sequence provided herein will provide guidance to those employing computer modeling techniques in place of or in addition to x-ray crystallography.

### Angiozenic Disorders to be Treated

The PA polypeptides, or agonists or antagonists thereto, that have activity in the cardiovascular, angiogenic, and endothelial assays described herein, and/or whose gene product has been found to be localized to the cardiovascular system, are likely to have therapeutic uses in a variety of angiogenic disorders, including systemic disorders that affect vessels, such as diabetes mellitus. Their therapeutic utility could include diseases of the arteries, capillaries, veins, and/or lymphatics. Examples of treatments hereunder include treating muscle wasting disease, treating osteoporosis, aiding in implant fixation to stimulate the growth of cells around the implant and therefore facilitate its attachment to its intended site, increasing IGF stability in tissues or in serum, if applicable, and increasing binding to the IGF receptor (since IGF has been shown in vitro to enhance human marrow erythroid and granulocytic progenitor cell growth).

The PA polypeptides or agonists or antagonists thereto may also be employed to stimulate erythropoiesis or granulopoiesis, to stimulate wound healing or tissue regeneration and associated therapies concerned with re-growth of tissue, such as connective tissue, skin, bone, cartilage, muscle, lung, or kidney, to promote angiogenesis, to stimulate or inhibit migration of endothelial cells, and to proliferate the growth of vascular smooth muscle and endothelial cell production. The increase in angiogenesis mediated by the PA polypeptide or antagonist would be beneficial to ischemic tissues and to collateral coronary development in the heart subsequent to coronary stenosis. Antagonists are used to inhibit the action of such polypeptides, for example, to limit the production of excess connective tissue during wound healing or pulmonary fibrosis if the PA polypeptide promotes such production. This would include treatment of acute myocardial infarction and heart failure.

Moreover, the present invention concerns the treatment of cardiac hypertrophy, regardless of the underlying cause, by administering a therapeutically effective dose of the PA polypeptide, or agonist or antagonist thereto. If the objective is the treatment of human patients, the PA polypeptide preferably is recombinant human PA polypeptide (rhPA or rhPA polypeptide). The treatment for cardiac hypertrophy can be performed at any of its various stages, which may result from a variety of diverse pathologic conditions, including myocardial infarction, hypertension, hypertrophic cardiomyopathy, and valvular regurgitation. The treatment extends to all stages of the progression of cardiac hypertrophy, with or without structural damage of the heart muscle, regardless of the underlying cardiac disorder.

The decision of whether to use the molecule itself or an agonist thereof for any particular indication, as opposed to an antagonist to the molecule, would depend mainly on whether the molecule herein promotes cardiovascularization, genesis of endothelial cells, or angiogenesis or inhibits these conditions. For example, if the molecule promotes angiogenesis, an antagonist thereof would be useful for treatment of disorders where it is desired to limit or prevent angiogenesis. Examples of such disorders include vascular tumors such as haemangioma, tumor angiogenesis, neovascularization in the retina, choroid, or cornea, associated with diabetic retinopathy or premature infant retinopathy or macular degeneration, and proliferative vitreoretinopathy, rheumatoid arthritis, Crohn's disease, atherosclerosis, ovarian hyperstimulation, psoriasis, endometriosis associated with neovascularization, restenosis subsequent to balloon angioplasty, scar tissue overproduction, for example, that seen in a keloid that forms after surgery, fibrosis after myocardial infarction, or fibrotic lesions associated with pulmonary fibrosis.

If, however, the molecule inhibits angiogenesis, it would be expected to be used directly for treatment of the above conditions.

On the other hand, if the molecule stimulates angiogenesis it would be used itself (or an agonist thereof) for indications where angiogenesis is desired such as peripheral vascular disease, hypertension, inflammatory vasculitides, Reynaud's disease and Reynaud's phenomenon, aneurysms, arterial restenosis, thrombophlebitis, lymphangitis, lymphedema, wound healing and tissue repair, ischemia reperfusion injury, angina, myocardial infarctions such as acute myocardial infarctions, chronic heart conditions, heart failure such as congestive heart failure, and osteoporosis.

If, however, the molecule inhibits angiogenesis, an antagonist thereof would be used for treatment of those conditions where angiogenesis is desired.

Specific types of diseases are described below, where the PA polypeptide herein or antagonists thereof may serve as useful for vascular-related drug targeting or as therapeutic targets for the treatment or prevention of the disorders. Atherosclerosis is a disease characterized by accumulation of plaques of initial thickening in arteries, due to accumulation of lipids, proliferation of smooth muscle cells, and formation of fibrous tissue within the arterial wall. The disease can affect large, medium, and small arteries in any organ. Changes in endothelial and vascular smooth muscle cell function are known to play an important role in modulating the accumulation and regression of these plaques.

Hypertension is characterized by raised vascular pressure in the systemic arterial, pulmonary arterial, or portal venous systems. Elevated pressure may result from or result in impaired endothelial function and/or vascular disease.

Inflammatory vasculitides include giant cell arteritis, Takayasu's arteritis, polyarteritis nodosa (including the microangiopathic form), Kawasaki's disease, microscopic polyangiitis, Wegener's granulomatosis, and a variety of infectious-related vascular disorders (including Henoch-Schonlein prupura). Altered endothelial cell function has been shown to be important in these diseases.

Reynaud's disease and Reynaud's phenomenon are characterized by intermittent abnormal impairment of the circulation through the extremities on exposure to cold. Altered endothelial cell function has been shown to be important in this disease.

Aneurysms are saccular or fusiform dilatations of the arterial or venous tree that are associated with altered endothelial cell and/or vascular smooth muscle cells.

Arterial restenosis (restenosis of the arterial wall) may occur following angioplasty as a result of alteration in the function and proliferation of endothelial and vascular smooth muscle cells.

Thrombophlebitis and lymphangitis are inflammatory disorders of veins and lymphatics, respectively, that may result from, and/or in, altered endothelial cell function. Similarly, lymphedema is a condition involving impaired lymphatic vessels resulting from endothelial cell function.

The family of benign and malignant vascular tumors are characterized by abnormal proliferation and growth of cellular elements of the vascular system. For example, lymphangiomas are benign tumors of the lymphatic system that are congenital, often cystic, malformations of the lymphatics that usually occur in newborns. Cystic tumors tend to grow into the adjacent tissue. Cystic tumors usually occur in the cervical and axillary region. They can also occur in the soft tissue of the extremities. The main symptoms are dilated, sometimes reticular, structured lymphatics and lymphocysts surrounded by connective tissue. Lymphangiomas are assumed to be caused by improperly connected embryonic lymphatics or their deficiency. The result is impaired local lymph drainage. Griener et al., Lymphology, 4: 140-144 (1971).

Another use for the PA polypeptides herein or antagonists thereto is in the prevention of tumor angiogenesis, which promotes vascularization of a tumor to enable it to growth and/or metastasize. This process is dependent on the growth of new blood vessels. Examples of neoplasms and related conditions that involve tumor angiogenesis include breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome.

Age-related macular degeneration (AMD) is a leading cause of severe visual loss in the elderly population. The exudative form of AMD is characterized by choroidal neovascularization and retinal pigment epithelial cell detachment. Because choroidal neovascularization is associated with a dramatic worsening in prognosis, the PA polypeptides or antagonist thereto is expected to be useful in reducing the severity of AMD.

Healing of trauma such as wound healing and tissue repair is also a targeted use for the PA polypeptides herein or their antagonists. Formation and regression of new blood vessels is essential for tissue healing and repair. This category includes bone, cartilage, tendon, ligament, and/or nerve tissue growth or regeneration, as well as wound healing and tissue repair and replacement, and in the treatment of burns, incisions, and ulcers. A PA polypeptide or antagonist thereof that induces cartilage and/or bone growth in circumstances where bone is not normally formed has application in the healing of bone fractures and cartilage damage or defects in humans and other animals. Such a preparation employing a PA polypeptide or antagonist thereof may have prophylactic use in closed as well as open fracture reduction and also in the improved fixation of artificial joints. De novo bone formation induced by an osteogenic agent contributes to the repair of congenital, trauma-induced, or oncologic, resection-induced craniofacial defects, and also is useful in cosmetic plastic surgery.

PA polypeptides or antagonists thereto may also be useful to promote better or faster closure of non-healing wounds, including without limitation pressure ulcers, ulcers associated with vascular insufficiency, surgical and traumatic wounds, and the like.

It is expected that a PA polypeptide or antagonist thereto may also exhibit activity for generation or regeneration of other tissues, such as organs (including, for example, pancreas, liver, intestine, kidney, skin, or endothelium), muscle (smooth, skeletal, or cardiac), and vascular (including vascular endothelium) tissue, or for promoting the growth of cells comprising such tissues. Part of the desired effects may be by inhibition or modulation of fibrotic scarring to allow normal tissue to regenerate.

A PA polypeptide herein or antagonist thereto may also be useful for gut protection or regeneration and treatment of lung or liver fibrosis, reperfusion injury in various tissues, and conditions resulting from systemic cytokine damage. Also, the PA polypeptide or antagonist thereto may be useful for promoting or inhibiting differentiation of tissues described above from precursor tissues or cells, or for inhibiting the growth of tissues described above.

A PA polypeptide or antagonist thereto may also be used in the treatment of periodontal diseases and in other tooth-repair processes. Such agents may provide an environment to attract bone-forming cells, stimulate growth of bone-forming cells, or induce differentiation of progenitors of bone-forming cells. A PA polypeptide herein or an antagonist thereto may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes, since blood vessels play an important role in the regulation of bone turnover and growth.

Another category of tissue regeneration activity that may be attributable to the PA polypeptide herein or antagonist thereto is tendon/ligament formation. A protein that induces tendon/ligament-like tissue or other tissue formation in circumstances where such tissue is not normally formed has application in the healing of tendon or ligament tears, deformities, and other tendon or ligament defects in humans and other animals. Such a preparation may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the PA polypeptide herein or antagonist thereto contributes to the repair of congenital, trauma-induced, or other tendon or ligament defects of other origin, and is also useful in cosmetic plastic surgery for attachment or repair of tendons or ligaments. The compositions herein may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions herein may also be useful in the treatment of tendinitis, carpal tunnel syndrome, and other tendon or ligament defects. The compositions may also include an appropriate matrix and/or sequestering agent as a carrier as is well known in the art.

The PA polypeptide or its antagonist may also be useful for proliferation of neural cells and for regeneration of nerve and brain tissue, i.e., for the treatment of central and peripheral nervous system disease and neuropathies, as well as mechanical and traumatic disorders, that involve degeneration, death, or trauma to neural cells or nerve tissue. More specifically, a PA polypeptide or its antagonist may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions that may be treated in accordance with the present invention include mechanical and traumatic disorders, such as spinal cord disorders, head trauma, and cerebrovascular diseases such as stroke. Peripheral neuropathies resulting from chemotherapy or other medical therapies may also be treatable using a PA polypeptide herein or antagonist thereto.

Ischemia-reperfusion injury is another indication. Endothelial cell dysfunction may be important in both the initiation of, and in regulation of the sequelae of events that occur following ischemia-reperfusion injury.

Rheumatoid arthritis is a further indication. Blood vessel growth and targeting of inflammatory cells through the vasculature is an important component in the pathogenesis of rheumatoid and sero-negative forms of arthritis.

A PA polypeptide or its antagonist may also be administered prophylactically to patients with cardiac hypertrophy, to prevent the progression of the condition, and avoid sudden death, including death of asymptomatic patients. Such preventative therapy is particularly warranted in the case of patients diagnosed with massive left ventricular cardiac hypertrophy (a maximal wall thickness of 35 mm or more in adults, or a comparable value in children), or in instances when the hemodynamic burden on the heart is particularly strong.

A PA polypeptide or its antagonist may also be useful in the management of atrial fibrillation, which develops in a substantial portion of patients diagnosed with hypertrophic cardiomyopathy.

Further indications include angina, myocardial infarctions such as acute myocardial infarctions, and heart failure such as congestive heart failure. Additional non-neoplastic conditions include psoriasis, diabetic and other proliferative retinopathies including retinopathy of prematurity, retrolental fibroplasia, neovascular glaucoma, thyroid hyperplasias (including Grave's disease), corneal and other tissue transplantation, chronic inflammation, lung inflammation, nephrotic syndrome, preeclampsia, ascites, pericardial effusion (such as that associated with pericarditis), and pleural effusion.

In view of the above, the PA polypeptides or agonists or antagonists thereof described herein, which are shown to alter or impact endothelial cell function, proliferation, and/or form, are likely to play an important role in the etiology and pathogenesis of many or all of the disorders noted above, and as such can serve as therapeutic targets to augment or inhibit these processes or for vascular-related drug targeting in these disorders.

### Administration Protocols, Schedules, Doses, and Formulations

The molecules herein and agonists and antagonists thereto are pharmaceutically useful as a prophylactic and therapeutic agent for various disorders and diseases as set forth above.

Therapeutic compositions of the PA polypeptides or agonists or antagonists are prepared for storage by mixing the desired molecule having the appropriate degree of purity with optional pharmaceutically acceptable carriers, excipients, or stabilizers (Remington's Pharmaceutical Sciences, 16th edition, Osol, A. ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of antagonist include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. The PA polypeptides or agonists or antagonists will typically be formulated in such vehicles at a concentration of about 0.1 mg/ml to 100 mg/ml.

Another formulation comprises incorporating a PA polypeptide or antagonist thereof into formed articles. Such articles can be used in modulating endothelial cell growth and angiogenesis. In addition, tumor invasion and metastasis may be modulated with these articles.

PA polypeptide or antagonist to be used for in vivo administration must be sterile. This is readily accomplished by filtration through sterile filtration membranes, prior to or following lyophilization and reconstitution. PA polypeptide ordinarily will be stored in lyophilized form or in solution if administered systemically. If in lyophilized form, PA polypeptide or antagonist thereto is typically formulated in combination with other ingredients for reconstitution with an appropriate diluent at the time for use. An example of a liquid formulation of PA polypeptide or antagonist is a sterile, clear, colorless unpreserved solution filled in a single-dose vial for subcutaneous injection. Preserved pharmaceutical compositions suitable for repeated use may contain, for example, depending mainly on the indication and type of polypeptide: a) a PA polypeptide or agonist or antagonist thereto; b) a buffer capable of maintaining the pH in a range of maximum stability of the polypeptide or other molecule in solution, preferably about 4-8; c) a detergent/surfactant primarily to stabilize the polypeptide or molecule against agitation-induced aggregation; d) an isotonifier; e) a preservative selected from the group of phenol, benzyl alcohol and a benzethonium halide, e.g., chloride; and f) water.

If the detergent employed is non-ionic, it may, for example, be polysorbates (e.g., POLYSORBATE™ (TWEEN™) 20, 80, etc.) or poloxamers (e.g., POLOXAMER™ 188). The use of non-ionic surfactants permits the formulation to be exposed to shear surface stresses without causing denaturation of the polypeptide. Further, such surfactant-containing formulations may be employed in aerosol devices such as those used in a pulmonary dosing, and needleless jet injector guns (see, e.g., EP 257,956).

An isotonifier may be present to ensure isotonicity of a liquid composition of the PA polypeptide or antagonist thereto, and includes polyhydric sugar alcohols, preferably trihydric or higher sugar alcohols, such as glycerin, erythritol, arabitol, xylitol, sorbitol, and mannitol. These sugar alcohols can be used alone or in combination. Alternatively, sodium chloride or other appropriate inorganic salts may be used to render the solutions isotonic.

The buffer may, for example, be an acetate, citrate, succinate, or phosphate buffer depending on the pH desired. The pH of one type of liquid formulation of this invention is buffered in the range of about 4 to 8, preferably about physiological pH.

The preservatives phenol, benzyl alcohol and benzethonium halides, e.g., chloride, are known antimicrobial agents that may be employed.

Therapeutic PA polypeptide compositions generally are placed into a container having a sterile access port, for example, an intravenous solution bag or vial having a stopper pierceable by a hypodermic injection needle. The formulations are preferably administered as repeated intravenous (i.v.), subcutaneous (s.c.), or intramuscular (i.m.) injections, or as aerosol formulations suitable for intranasal or intrapulmonary delivery (for intrapulmonary delivery see, e.g., EP 257,956).

The PA polypeptide can also be administered in the form of sustained-released preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al, J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they may denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Sustained-release PA polypeptide compositions also include liposomally entrapped PA polypeptides. Liposomes containing the PA polypeptide are prepared by methods known per se: DE 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamellar type in which the lipid content is greater than about 30 mol. % cholesterol, the selected proportion being adjusted for the optimal therapy.

The therapeutically effective dose of PA polypeptide or antagonist thereto will, of course, vary depending on such factors as the pathological condition to be treated (including prevention), the method of administration, the type of compound being used for treatment, any co-therapy involved, the patient's age, weight, general medical condition, medical history, etc., and its determination is well within the skill of a practicing physician. Accordingly, it will be necessary for the therapist to titer the dosage and modify the route of administration as required to obtain the maximal therapeutic effect. If the PA polypeptide has a narrow host range, for the treatment of human patients formulations comprising human PA polypeptide, more preferably native-sequence human PA polypeptide, are preferred. The clinician will administer PA polypeptide until a dosage is reached that achieves the desired effect for treatment of the condition in question. For example, if the objective is the treatment of CHF, the amount would be one that inhibits the progressive cardiac hypertrophy associated with this condition. The progress of this therapy is easily monitored by echo cardiography. Similarly, in patients with hypertrophic cardiomyopathy, PA polypeptide can be administered on an empirical basis.

With the above guidelines, the effective dose generally is within the range of from about 0.001 to about 1.0 mg/kg, more preferably about 0.01-1.0 mg/kg, most preferably about 0.01-0.1 mg/kg.

For non-oral use in treating human adult hypertension, it is advantageous to administer PA polypeptide in the form of an injection at about 0.01 to 50 mg, preferably about 0.05 to 20 mg, most preferably 1 to 20 mg, per kg body weight, 1 to 3 times daily by intravenous injection. For oral administration, a molecule based on the PA polypeptide is preferably administered at about 5 mg to 1 g, preferably about 10 to 100 mg, per kg body weight, 1 to 3 times daily. It should be appreciated that endotoxin contamination should be kept minimally at a safe level, for example, less than 0.5 ng/mg protein. Moreover, for human administration, the formulations preferably meet sterility, pyrogenicity, general safety, and purity as required by FDA Office and Biologics standards.

The dosage regimen of a pharmaceutical composition containing PA polypeptide to be used in tissue regeneration will be determined by the attending physician considering various factors that modify the action of the polypeptides, e.g., amount of tissue weight desired to be formed, the site of damage, the condition of the damaged tissue, the size of a wound, type of damaged tissue (e.g., bone), the patient's age, sex, and diet, the severity of any infection, time of administration, and other clinical factors. The dosage may vary with the type of matrix used in the reconstitution and with inclusion of other proteins in the pharmaceutical composition. For example, the addition of other known growth factors, such as IGF-I, to the final composition may also affect the dosage. Progress can be monitored by periodic assessment of tissue/bone growth and/or repair, for example, X-rays, histomorphometric determinations, and tetracycline labeling.

The route of PA polypeptide or antagonist or agonist administration is in accord with known methods, e.g., by injection or infusion by intravenous, intramuscular, intracerebral, intraperitoneal, intracerobrospinal, subcutaneous, intraocular, intraarticular, intrasynovial, intrathecal, oral, topical, or inhalation routes, or by sustained-release systems as noted below. The PA polypeptide or antagonists thereof also are suitably administered by intratumoral, peritumoral, intralesional, or perilesional routes, to exert local as well as systemic therapeutic effects. The intraperitoneal route is expected to be particularly useful, for example, in the treatment of ovarian tumors.

If a peptide or small molecule is employed as an antagonist or agonist, it is preferably administered orally or non-orally in the form of a liquid or solid to mammals.

Examples of pharmacologically acceptable salts of molecules that form salts and are useful hereunder include alkali metal salts (e.g., sodium salt, potassium salt), alkaline earth metal salts (e.g., calcium salt, magnesium salt), ammonium salts, organic base salts (e.g., pyridine salt, triethylamine salt), inorganic acid salts (e.g., hydrochloride, sulfate, nitrate), and salts of organic acid (e.g., acetate, oxalate, p-toluenesulfonate).

For compositions herein that are useful for bone, cartilage, tendon, or ligament regeneration, the therapeutic method includes administering the composition topically, systemically, or locally as an implant or device. When administered, the therapeutic composition for use is in a pyrogen-free, physiologically acceptable form. Further, the composition may desirably be encapsulated or injected in a viscous form for delivery to the site of bone, cartilage, or tissue damage. Topical administration may be suitable for wound healing and tissue repair. Preferably, for bone and/or cartilage formation, the composition would include a matrix capable of delivering the protein-containing composition to the site of bone and/or cartilage damage, providing a structure for the developing bone and cartilage and preferably capable of being resorbed into the body. Such matrices may be formed of materials presently in use for other implanted medical applications.

The choice of matrix material is based on biocompatibility, biodegradability, mechanical properties, cosmetic appearance, and interface properties. The particular application of the compositions will define the appropriate formulation. Potential matrices for the compositions may be biodegradable and chemically defined calcium sulfate, tricalcium phosphate, hydroxyapatite, polylactic acid, polyglycolic acid, and polyanhydrides. Other potential materials are biodegradable and biologically well-defined, such as bone or dermal collagen. Further matrices are comprised of pure proteins or extracellular matrix components. Other potential matrices are nonbiodegradable and chemically defined, such as sintered hydroxyapatite, bioglass, aluminates, or other ceramics. Matrices may be comprised of combinations of any of the above-mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

One specific embodiment is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the polypeptide compositions from disassociating from the matrix.

One suitable family of sequestering agents is cellulosic materials such as alkylcelluloses (including hydroxyalkylcelluloses), including methylcellulose, ethylcellulose, hydoxyethylcellulose, hydroxypropylcellulose, hydroxypropylmethylcellulose, and carboxymethylcellulose, one preferred being cationic salts of carboxymethylcellulose (CMC).

Other preferred sequestering agents include hyaluronic acid, sodium alginate, poly(ethylene glycol), polyoxyethylene oxide, carboxyvinyl polymer, and poly(vinyl alcohol). The amount of sequestering agent useful herein is 0.5-20 wt %, preferably 1-10 wt %, based on total formulation weight, which represents the amount necessary to prevent desorption of the polypeptide (or its antagonist) from the polymer matrix and to provide appropriate handling of the composition, yet not so much that the progenitor cells are prevented from infiltrating the matrix, thereby providing the polypeptide (or its antagonist) the opportunity to assist the osteogenic activity of the progenitor cells.

### Combination Therapies

The effectiveness of the PA polypeptide or an agonist or antagonist thereof in preventing or treating the disorder in question may be improved by administering the active agent serially or in combination with another agent that is effective for those purposes, either in the same composition or as separate compositions.

For example, for treatment of cardiac hypertrophy, PA polypeptide therapy can be combined with the administration of inhibitors of known cardiac myocyte hypertrophy factors, e.g., inhibitors of α-adrenergic agonists such as phenylephrine; endothelin-1 inhibitors such as BOSENTAN™ and MOXONODIN™; inhibitors to CT-1 (U.S. Pat. No. 5,679,545); inhibitors to LIF; ACE inhibitors; des-aspartate-angiotensin I inhibitors (U.S. Pat. No. 5,773,415), and angiotensin II inhibitors.

For treatment of cardiac hypertrophy associated with hypertension, PA polypeptide can be administered in combination with β-adrenergic receptor blocking agents, e.g., propranolol, timolol, tertalolol, carteolol, nadolol, betaxolol, penbutolol, acetobutolol, atenolol, metoprolol, or carvedilol; ACE inhibitors, e.g., quinapril, captopril, enalapril, ramipril, benazepril, fosinopril, or lisinopril; diuretics, e.g., chlorothiazide, hydrochlorothiazide, hydroflumethazide, methylchlothiazide, benzthiazide, dichlorphenamide, acetazolamide, or indapamide; and/or calcium channel blockers, e.g., diltiazem, nifedipine, verapamil, or nicardipine. Pharrnaceutical compositions comprising the therapeutic agents identified herein by their generic names are commercially available, and are to be administered following the manufacturers' instructions for dosage, administration, adverse effects, contraindications, etc. See, e. j., Physicians' Desk Reference (Medical Economics Data Production Co.: Montvale, N.J., 1997), 51st Edition. Preferred candidates for combination therapy in the treatment of hypertrophic cardiomyopathy are β-adrenergic-blocking drugs (e.g., propranolol, timolol, tertalolol, carteolol, nadolol, betaxolol, penbutolol, acetobutolol, atenolol, metoprolol, or carvedilol), verapamil, difedipine, or diltiazem. Treatment of hypertrophy associated with high blood pressure may require the use of antihypertensive drug therapy, using calcium channel blockers, e.g., diltiazem, nifedipine, verapamil, or nicardipine; β-adrenergic blocking agents; diuretics, e.g., chlorothiazide, hydrochlorothiazide, hydroflumethazide, methylchlothiazide, benzthiazide, dichlorphenamide, acetazolamide, or indapamide; and/or ACE-inhibitors, e.g., quinapril, captopril, enalapril, ramipril, benazepril, fosinopril, or lisinopril.

For other indications, PA polypeptides or their antagonists may be combined with other agents beneficial to the treatment of the bone and/or cartilage defect, wound, or tissue in question. These agents include various growth factors such as EGF, PDGF, TGF- or TGF-, IGF, FGF, and CTGF.

In addition, PA polypeptides or their antagonists used to treat cancer may be combined with cytotoxic, chemotherapeutic, or growth-inhibitory agents as identified above. Also, for cancer treatment, the PA polypeptide or antagonist thereof is suitably administered serially or in combination with radiological treatments, whether involving irradiation or administration of radioactive substances.

The effective amounts of the therapeutic agents administered in combination with the PA polypeptide or antagonist thereof will be at the physician's or veterinarian's discretion. Dosage administration and adjustment is done to achieve maximal management of the conditions to be treated For example, for treating hypertension, these amounts ideally take into account use of diuretics or digitalis, and conditions such as hyper- or hypotension, renal impairment, etc. The dose will additionally depend on such factors as the type of the therapeutic agent to be used and the specific patient being treated. Typically, the amount employed will be the same dose as that used, if the given therapeutic agent is administered without PA polypeptide.

### Articles of Manufacture

An article of manufacture such as a kit containing PA polypeptide or antagonists thereof useful for the diagnosis or treatment of the disorders described above comprises at least a container and a label. Suitable containers include, for example, bottles, vials, syringes, and test tubes. The containers may be formed from a variety of materials such as glass or plastic. The container holds a composition that is effective for diagnosing or treating the condition and may have a sterile access port (for example, the container may be an intravenous solution bag or a vial having a stopper pierceable by a hypodermic injection needle). The active agent in the composition is the PA polypeptide or an agonist or antagonist thereto. The label on, or associated with, the container indicates that the composition is used for diagnosing or treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use. The article of manufacture may also comprise a second or third container with another active agent as described above.

The invention will be further described in the following examples, which do not limit the scope of the invention described in the claims. The following examples are offered for illustrative purposes only, and are not intended to limit the scope of the present invention in any way.

All commercially available reagents referred to in the examples were used according to manufacturer's instructions unless otherwise indicated. The source of those cells identified by ATCC accession numbers in the following examples, and throughout the specification, is the American Type Culture Collection, Manassas, Va.

### EXAMPLE 1

### Quantitative Expression Analysis

QEA is a method for rapid detection and quantitation of gene expression in tissue or cells. This process uses a proprietary process called Gene Calling™ that can identify, in a quantitative fashion, all the various mRNA transcripts differentially expressed in an RNA sample originating from cells or tissue.

The QEA GeneCalling™ method involves the preparation of total RNA followed by mRNA purification and double stranded cDNA synthesis. This is followed by digestion of cDNA with restriction enzyme pairs to produce cDNA fragments that are ligated with linkers. Primer pairs bearing the specific sequences of the linkers used are then used to amplify the products in a PCR reaction.

HUVECs grown in collagen gel were lysed in Trizol reagent and total RNA was prepared according to Trizol recommended procedure. Poly-A⁺ RNA was prepared by fractionation of total RNA using biotinylated oligo-dt₍₂₅₎/Streptavidin magnetic bead method (MPG, Lincoln Park, N.J.). cDNA was prepared by reverse transcription of oligo-dt primed mRNA followed by second strand synthesis.

Double-stranded cDNA is then digested with pairs of restriction enzymes with 6 base-pair recognition sites. More than 48 enzyme pairs were used, these were chosen such that a representative coverage of the majority of possible sequences in a given cDNA sample is achieved.

Where possible, GeneCalling™ results were confirmed using the TaqMan™ procedure. TaqMan™ is a PCR-based method for measurement of gene expression. Gene specific PCR oligonucleotide primer pairs and an oligonucleotide probe labeled with a reporter fluorescent dye at the 5′ end and quencher fluorescent dye at the 3′ end were designed using the Oligo 4.0 software (National Bioscience, Plymouth Minn.). Total RNA (50 ng) was added to a 50 μl RT-PCR reaction mixture according to the manufacturer's protocol (Roche Molecular Systems Inc. Branchburg, N.J.). The thermal cycling conditions included 1 cycle at 48° C. for 30 min, 1 cycle at 95° C. for 10 min, 40 cycles at 95° C. for 15 s, annealing at 60° C. for 1 min, and a final hold at 25° for 2 min. Standard curves for the expression of each gene were generated by serial dilution of a standard preparation of total RNA isolated from quiescent HUVECs grown in monolayer culture. Data are expressed as the fold induction normalized to the same gene from quiescent HUVEC RNA. For comparison data obtained with RNA extracted from HUVECs grown on collagen film is included. HUVECs grown on collagen film do not undergo differentiation into tube-like structures.

### EXAMPLE 2

### Isolation of cDNA Clones Encoding a Human PA

Formation of Three-Dimensional Collagen Gels:

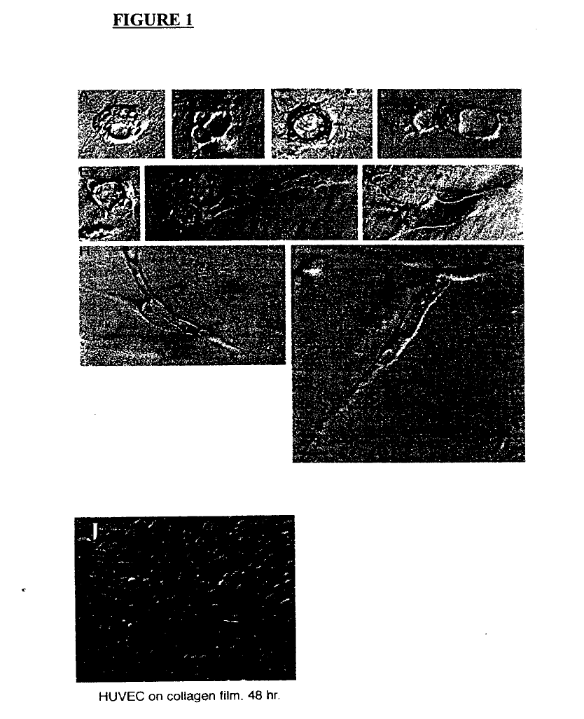

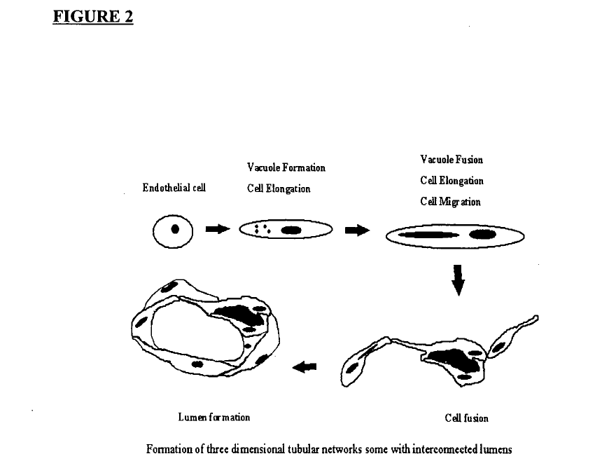

Tube formation by endothelial cells is a critical process in the development of a blood vessel during angiogenesis and vasculogenesis. Human umbilical cord endothelial cells (HUVEC) undergoing tube formation in collagen gels in the presence of growth factors, mimic the angiogenic environment of endothelial cells in vivo, providing a well-accepted system for angiogenesis and vasculogenesis, both in normal and neoplastic conditions. The three dimensional gel is a prerequisite for the differentiation and fusion of endothelial cells into tubes. HUVECs grown on the surface of gelatin gels or on plastic do not undergo tube-formation. (See FIG. 1, Panels A-J, and FIG. 2) In brief, HUVECs were grown under various conditions, inductive or non-inductive to tube formation, either on collagen film (non-inductive) or in collagen gels (inductive), with or without the addition of growth factors to simulate normal angiogenic- or tumor-derived factors. Differential cDNA screening was used to identify genes critical to this process. The particular method used to quantitate endothelial cell gene expression was Quantitative Expression Analysis (QEA, U.S. Pat. No. 5,871,697). HUVEC total RNA was prepared followed by mRNA purification and double stranded cDNA synthesis. The cDNA was digested with restriction enzyme pairs to produce cDNA fragments, which were then ligated with linkers. Primer pairs bearing the specific sequences of the linkers were used to amplify the products in a PCR reaction. Quantification and identification of amplified products revealed modulated genes, thus identifying genes critical to angiogeneisis.

Tube Formation.

Collagen gels were formed by mixing together an ice-cold gelation solution (10× M199, water, 0.53 M NaHCO3, 200 mM L-glutamine, type I collagen, 0.1M NaOH; 100:27.7:50:10:750:62.5 by volume) and cells in 1× basal medium at a concentration of 3×10⁶ cells/ml at a ratio of 4 volumes gelation solution: 1 volume of cells. The gels were allowed to form by incubation in a CO2-free incubator at 37 C for 30 min-1 hr. The gels were then overlaid with 1× Basal medium consisting of M199 supplemented with 1% FBS, 1× ITS, 2 mM L-glutamine, 50 mg/ml ascorbic acid, 26.5 mM NaHCO3, 100 U/ml penicillin and 100 U/ml streptomycin. In the tube-forming experiments, the culture media was supplemented with 80 nM PMA, 40 ng/ml bFGF and 40 ng/ml VEGF. In parallel set of experiments, endothelial cells were cultured on the surface of type I collagen, or on pig skin gelatin (Difco) in 1× Basal medium consisting of M199 supplemented with 1% FBS, 1× ITS, 2 mM L-glutamine, 50 mg/ml ascorbic acid, 26.5 mM NaHCO3, 100 U/ml penicillin and 100 U/ml streptomycin without or with 80 nM PMA, 40 ng/ml bFGF and 40 ng/ml VEGF. For the GeneCalling experiment, mRNA was isolated from cells incubated in the above conditions for 4 hr, 24 hr and 48 hr.

mRNA Isolation and cDNA Synthesis

Media was aspirated from the surface of the collagen gels and the gels scraped into a 50 ml polypropylene tube containing 3 volumes of Tri-Reagent-LS (Molecular Research Center, Cincinnati, Ohio). Cells grown in the tubes were incubated 10 min at 23° C. with intermittent gentle agitation. The tubes were stored at −80° C. until all experimental samples had been collected. The tubes were then thawed at room temperature and the mRNA extracted following manufacturer's specifications. The RNA pellets were resuspended in DEPC-treated water and RNA content quantified spectroscopically at 260 nm. RNA samples were stored −20° C. Samples used for GeneCalling™ analysis were shipped on dry ice to Curagen (New Haven, Conn.). Samples from time points of 4, 24 and 48 hrs were used for the GeneCalling™ analysis, and in a separate experiments, samples cells grown in collagen gels and on the surface of type I collage in 1× basal medium supplemented with 80 nM PMA, 40 ng/ml bFGF and 40 ng/ml VEGF from time points of 30 min, 2, 4, 8, 16, 24, 38 and 46.5 hrs were prepared for Taqman confirmation.

For the quantitative expression analysis, contaminating DNA was removed by treatment of the isolated RNA with DNAse I (Promega, Madison, Wis.). Poly-A+ RNA was prepared by fractionation of total RNA using an mRNA purification kit that utilized the biotinylated oligo-dT-Streptavidin magnetic bead method (MPG, LincolnPark, N.J.) followed by cDNA synthesis by reverse transcription of oligo-dT primed mRNA (Superscript II, Life Technologies) and second strand synthesis. Terminal phosphate removal is achieved by treatment with Artic Shrimp Alkaline Phosphtase (Amersham Life Sciences, Piscataway, N.J.) followed by purification of cDNA by phenol-chloroform extraction. Yield of cDNA was quantitated by fluorometry using PicoGreen dye (Molecular Probes, Eugene Oreg.). Double stranded DNA was digested using pairs of restriction enzymes with 6 base-pair recognition sites. More than 48 enzyme pairs were used and were chosen such that a representative coverage of most of the possible sequences in a given DNA sample was achieved. PCR amplification using specific linkers was carried out as described previously. The final DNA products were denatured by heating to 96 C and electrophoresed on ultra-thin polyacrylamide gels under denaturing conditions in 6M urea. PCR products were visualized by the presence of FAM label on the product using a multi-color laser excitation Niagara (Curagen, New Haven Conn.) imaging system.

Data Interpretation

GeneCalling™ uses a fully integrated Web-based interactive bioinformatics data gathering and analysis suite called “GeneScape.” The data obtained from Niagara gels were GeneCalled against public and proprietary databases using present statistical and mathematical criteria and a gene list was generated from the cDNA fragment data that is a list of likely genes that the cDNA fragment can belong to based on the size of the fragment and the position of the restriction enzyme pair that produced it in the known sequence. If a gene candidate could not be obtained, the cDNA fragment was designated as belonging to a putative novel gene.

Confirmation of GeneCalls

A GeneCall is defined as the probability of a cDNA fragment belonging to a known gene. GeneCalls were confirmed in a poisoning reactions where the known sequence of the likely gene of interest is used to design poisoning primers as previously described (Shimkets et al., Nature Biotechnology). Ablation of the cDNA fragment of interest confirmed that the cDNA fragment belonged to the gene for which the primer was designed.

Novel cDNA Fragments

If no GeneCall was obtained for a cDNA fragment, the cDNA fragment was eluted from, and subcloned into E. coli using standard TA-cloning vector (Invitrogen, Palo Alto, Calif.). The cDNA fragment was then sequenced and the resulting sequence used to design poison primers for confirmation as described above.

Selection (Gating) of Differentially Expressed Genes

The experimental design was based on the observation that endothelial cells grown on the surface of type I collagen in 1× basal medium supplemented with 80 nM PMA, 40 ng/ml bFGF and 40 ng/ml VEGF do not form tubes, but rather remain as monolayer. This is also true if the cells are grown on gelatin, a form of denatured collagen. However, if the cells are suspended in a three dimensional collagen gel, and grown in 1× basal media supplemented with 80 nM PMA, 40 ng/ml bFGF and 40 ng/ml VEGF, the cells undergo a synchronous differentiation into an interconnected tube like network. The tubular structures contain lumen-like structures. At 4 hours, large intracellular vacuoles are forming, but the cells are still round. At 24 hrs, the cells have become elongated and many cells are touching other cells. By 48 hrs, the cells have become interconnected and share common lumens. To select for genes that play a role in this differentiation, an array of GeneCalling differences was set up such that cDNA fragments that changed more than 2 fold between 24 and 4, 48 and 4 or 48 and 4 hrs, in the 3-D gel environment, but which were unchanged or changed less than 2 fold in the 2D (surface of type I collagen or gelatin) environment at the same time comparisons were preferentially selected and identified. In addition, those cDNA fragments which demonstrate large (greater than 8 fold) changes in gene expression were also identified.

### EXAMPLE 3

### Use of PA DNA as a Hybridization Probe

The following method describes use of a nucleotide sequence encoding PA DNA as a hybridization probe.

DNA comprising the coding sequence of full-length or mature PA polypeptide is employed as a probe to screen for homologous DNAs (such as those encoding naturally-occurring variants of PA) in human tissue cDNA libraries or human tissue genomic libraries.

Hybridization and washing of filters containing either library DNAs is performed under the following high stringency conditions. Hybridization of radiolabeled PA-derived probe to the filters is performed in a solution of 50% formamide, 5×SSC, 0.1% SDS, 0.1% sodium pyrophosphate, 50 mM sodium phosphate, pH 6.8, 2× Denhardt's solution, and 10% dextran sulfate at 42° C. for 20 hours. Washing of the filters is performed in an aqueous solution of 0.1×SSC and 0.1% SDS at 42° C.

DNAs having a desired sequence identity with the DNA encoding full-length native sequence PA DNA can then be identified using standard techniques known in the art.

It is also possible to utilize fragments of PA DNA as probes in a similar procedure as above, or alternatively RNA transcripts made from DNA encoding PA polypeptide (or DNA fragments encoding portions of PA polypeptide) can likewise be used as probes. Preparation and use of such DNA fragments and RNA transcripts is well known in the art and can be performed by anyone skilled in the art.

### EXAMPLE 4

### Expression of a PA Gene in E. coli

This example illustrates preparation of an unglycosylated form of PA polypeptide by recombinant expression in E. coli.

The DNA sequence encoding PA polypeptide is initially amplified using selected PCR primers. The primers should contain restriction enzyme sites that correspond to the restriction enzyme sites on the selected expression vector. A variety of expression vectors can be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the PA coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture can subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized PA protein can then be purified using a metal chelating column under conditions that allow tight binding of the protein.

PA polypeptides can be expressed in E. coli in a poly-His tagged form, using the following procedure. The DNA encoding PA is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) Ion galE rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citrate.2H2O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.1 M and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 4° C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded PA polypeptide are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

### EXAMPLE 5

### Expression of PAP in Mammalian Cells

This example illustrates preparation of a potentially glycosylated form of PA polypeptide by recombinant expression in mammalian cells.

The vector, pRK5 (see EP 307,247, published Mar. 15, 1989), is employed as the expression vector. Optionally, the PA DNA is ligated into pRK5 with selected restriction enzymes to allow insertion of the PA DNA using ligation methods such as described in Sambrook et al., supra. The resulting vector is called pRK5-PA.

In one embodiment, the selected host cells can be HUVEC cells as described above, using the vectors and transfection methods described herein for other mammalian cells. Transfected HUVEC cells over-expressing PA gene or expressing PA antisense are tested, for example, in the tube formation assay.

In one embodiment, the selected host cells can be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-PA DNA is mixed with about 1 μg DNA encoding the VA RNA gene (Thimmappaya et al., Cell, 31:543 (1982)) and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml ³⁵S-cysteine and 200 μCi/ml ³⁵S-methionine. After a 12 hour incubation, the conditioned medium is collected, concentrated on a spin filter, and loaded onto a 15% SDS gel. The processed gel can be dried and exposed to film for a selected period of time to reveal the presence of PA polypeptide. The cultures containing transfected cells can undergo further incubation (in serum free medium) and the medium is then tested in selected bioassays.

In an alternative technique, PA can be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-PA DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0.1 μg/ml bovine transferrin. After about four days, the conditioned media is centrifuged and filtered to remove cells and debris. The sample containing expressed PA can then be concentrated and purified by any selected method, such as dialysis and/or column chromatography.

In another embodiment, PA DNA can be expressed in CHO cells. The pRK5-PA can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of PA polypeptide, the culture medium can be replaced with serum free medium. Preferably, the cultures are incubated for about 6 days, and then the conditioned medium is harvested. The medium containing the expressed PA can then be concentrated and purified by any selected method.

Epitope-tagged PA polypeptide can also be expressed in host CHO cells. The PA DNA can be subcloned out of the pRK5 vector. The subclone insert can undergo PCR to fuse in frame with a selected epitope tag such as a poly-his tag into a Baculovirus expression vector. The poly-his tagged PA polypeptide insert can then be subcloned into a SV40 driven vector containing a selection marker such as DHFR for selection of stable clones. Finally, the CHO cells can be transfected (as described above) with the SV40 driven vector. Labeling can be performed, as described above, to verify expression. The culture medium containing the expressed poly-His tagged PA polypeptide can then be concentrated and purified by any selected method, such as by Ni²⁺-chelate affinity chromatography.

PA can also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents Superfect® (Quiagen), Dosper® or Fugene® (Boehringer Mannheim). The cells are grown as described in Lucas et al., supra. Approximately 3×10⁻⁷ cells are frozen in an ampule for further growth and production as described below.

The ampules containing the plasmid DNA are thawed by placement into water bath and mixed by vortexing. The contents are pipetted into a centrifuge tube containing 10 mLs of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media can be employed, a production medium described in U.S. Pat. No. 5,122,469, issued Jun. 16, 1992 can actually be used. A 3 L production spinner is seeded at 1.2×10⁶ cells/mL. On day 0, the cell number pH ie determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Coming 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μL of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

### EXAMPLE 6

### Expression of a PA Gene in Yeast

Recombinant expression of PA polypeptide in yeast can also be accomplished.

First, yeast expression vectors are constructed for intracellular production or secretion of PA from the ADH2/GAPDH promoter. DNA encoding PA and the promoter is inserted into suitable restriction enzyme sites in the selected plasmid to direct intracellular expression of PA. For secretion, DNA encoding PA can be cloned into the selected plasmid, together with DNA encoding the ADH2/GAPDH promoter, a native PA signal peptide or other mammalian signal peptide, or, for example, a yeast alpha-factor or invertase secretory signal/leader sequence, and linker sequences (if needed) for expression of PA.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant PA polypeptide can subsequently be isolated and purified by removing the yeast cells from the fermentation medium by centrifugation and then concentrating the medium using selected cartridge filters. The concentrate containing PA polypeptide can further be purified using selected column chromatography resins.

### EXAMPLE 7

### Expression of a PA Polypeptide in Baculovirus-Infected Cells

The following method describes recombinant expression of PA polypeptidein Baculovirus-infected insect cells.

The DNA sequence coding for PA polypeptide is fused upstream of an epitope tag contained within a baculovirus expression vector. Such epitope tags include poly-his tags and immunoglobulin tags (like Fc regions of IgG). A variety of plasmids can be employed, including plasmids derived from commercially available plasmids such as pVL1393 (Novagen). Briefly, the sequence encoding PA polypeptide or the desired portion of the coding sequence of PA gene such as the sequence encoding the extracellular domain of a transmembrane protein or the sequence encoding the mature protein if the protein is extracellular is amplified by PCR with primers complementary to the 5′ and 3′ regions. The 5′ primer can incorporate flanking (selected) restriction enzyme sites. The product is then digested with those selected restriction enzymes and subcloned into the expression vector.

Recombinant baculovirus is generated by co-transfecting the above plasmid and BaculoGold™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-his tagged PA polypeptide can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl,; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged PA are pooled and dialyzed against loading buffer.

Alternatively, purification of the IgG tagged (or Fc tagged) PA can be performed using known chromatography techniques, including for instance, Protein A or protein G column chromatography.

### EXAMPLE 8

### Preparation of Antibodies that Bind a PA Polypeptide

This example illustrates preparation of monoclonal antibodies which can specifically bind PA.

Techniques for producing the monoclonal antibodies are known in the art and are described, for instance, in Goding, supra. Immunogens that can be employed include purified PA, fusion proteins containing PA, and cells expressing recombinant PA on the cell surface. Selection of the immunogen can be made by the skilled artisan without undue experimentation.

Mice, such as Balb/c, are immunized with the PA immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice can also be boosted with additional immunization injections. Serum samples can be periodically obtained from the mice by retro-orbital bleeding for testing in ELISA assays to detect anti-PA antibodies.

After a suitable antibody titer has been detected, the animals “positive” for antibodies can be injected with a final intravenous injection of PA polypeptide. Three to four days later, the mice are sacrificed and the spleen cells are harvested. The spleen cells are then fused (using 35% polyethylene glycol) to a selected murine myeloma cell line such as P3×63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells will be screened in an ELISA for reactivity against PA polypeptide. Determination of “positive” hybridoma cells secreting the desired monoclonal antibodies against PA polypeptide is within the skill in the art.

The positive hybridoma cells can be injected intraperitoneally into syngeneic Balb/c mice to produce ascites containing the anti-PA monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### EXAMPLE 9

### Purification of PA Polypeptides using Specific Antibodies

Native or recombinant PA polypeptides can be purified by a variety of standard techniques in the art of protein purification. For example, pro-PA polypeptide, mature PA polypeptide, or pre-PA polypeptide is purified by immunoaffinity chromatography using antibodies specific for the PA polypeptide of interest. In general, an immunoaffinity column is constructed by covalently coupling the anti-PA polypeptide antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of PA polypeptide by preparing a fraction from cells containing PA polypeptide in a soluble form. This preparation is derived by solubilization of the whole cell or of a subcellular fraction obtained via differential centrifugation by the addition of detergent or by other methods well known in the art. Alternatively, soluble PA polypeptide containing a signal sequence can be secreted in useful quantity into the medium in which the cells are grown.

A soluble PA polypeptide-containing preparation is passed over the immunoaffinity column, and the column is washed under conditions that allow the preferential absorbance of PA polypeptide (e.g., high ionic strength buffers in the presence of detergent). Then, the column is eluted under conditions that disrupt antibody/PA polypeptide binding (e.g., a low pH buffer such as approximately pH 2-3, or a high concentration of a chaotrope such as urea or thiocyanate ion), and PA polypeptide is collected.

### EXAMPLE 10

### Stimulation of Endothelial Tube Formation

This assay follows the assay described in Davis and Camarillo, Experimental Cell Research, 224:39-51 (1996), or one modified from it as follows:

Protocol: Human venous umbilical vein endothelial cells (HUVEC, Cell Systems) (passage number less than 8 from primary) are mixed with type I rat tail collagen, final concentration 2.6 mg/ml at a density of 6×105 cells/ml and plated at 50 μl per well on a 96-well plate. The gel is allowed to solidify for 1 hr at 37° C., then 50 μl per well of M199 culture media supplemented with 1% FBS and a PA polypeptide sample (at dilutions of 1%, 0.1%, and 0.01%, respectively) is added along with 1 μM 6-FAM-FITC dye to stain vacuoles while they are forming. Cells are incubated at 37° C./5% CO2 for 48 hrs, fixed with 3.7% formalin at room temperature for 10 minutes, washed with PBS five times, then stained with Rh-Phalloidin at 4 C overnight followed by nuclear staining with 4 μM DAPI.

1. abApoptosis Assay

This assay will identify factors that facilitate cell survival in a 3-dimensional matrix in the presence of exogenous growth factors (VEGF, bFGF without PMA).

A positive result is equal to or less than 1. 0=no apoptosis, 1=less than 20% cells are apoptotic, 2=less than 50% cells are apoptotic, 3=greater than 50% cells are apoptic. Stimulators of apoptosis in this system are expected to be apoptotic factors, and inhibitors are expected to prevent or lessen apoptosis.

2. abVacuoles Assay

This assay will identify factors that stimulate endothelial vacuole formation and lumen formation in the presence of bFGF and VEGF (40 ng/ml).

A positive result is equal to or greater than 2. 1=vacuoles present in less than 20% of cells, 2=vacuoles present in 20-50% of cells, 3=vacuoles present in greater than 50% of cells. This assay is designed to identify factors that are involved in stimulating pinocytosis, ion pumping, permeability, and junction formation.

3. abTube Formation Assay

This assay will identify factors that stimulate endothelial tube formation in a 3-dimensional matrix. This assay will identify factors that stimulate endothelial cells to differentiate into a tube-like structure in a 3-dimensional matrix in the presence of exogenous growth factors (VEGF, bFGF).

A positive result is equal to or greater than 2. 1=cells are all round, 2=cells are elongated, 3=cells are forming tubes with some connections, 4=cells are forming complex tubular networks. This assay would identify factors that may be involved in stimulating tracking, chemotaxis, or endothelial shape change.

The results clearly demonstrate that more complex tube formation occurs with PA-IgG and PA-poly-His samples at 1% dilution compared with buffer controls (10 mM HEPES/0.14M NaCl/4% mannitol, pH 6.8) at 1% dilution.

### EXAMPLE 11

### Stimulation of Endothelial Cell Proliferation

This assay is designed to determine whether PA shows the ability to stimulate adrenal cortical capillary endothelial cell (ACE) growth.

Bovine adrenal cortical capillary endothelial cells (ACE) (from primary culture, maximum of 12-14 passages) were plated in 96-well plates at 500 cells/well per 100 microliter. Assay media included low glucose DMEM, 10% calf serum, 2 mM glutamine, and 1× penicillin/streptomycin/fungizone. Control wells included the following: (1) no ACE cells added; (2) ACE cells alone; (3) ACE cells plus VEGF (5 ng/ml); and (4) ACE cells plus FGF (5 ng/ml). The control or test sample, (in 100 microliter volumes), was then added to the wells (at dilutions of 1%, 0.1% and 0.01%, respectively). The cell cultures were incubated for 6-7 days at 37° C./5% CO2. After the incubation, the media in the wells was aspirated, and the cells were washed 1× with PBS. An acid phosphatase reaction mixture (100 microliter; 0.1M sodium acetate, pH 5.5, 0.1% Triton X-100, 10 mM p-nitrophenyl phosphate) was then added to each well. After a 2 hour incubation at 37° C., the reaction was stopped by addition of 10 microliters 1N NaOH. Optical density (OD) was measured on a microplate reader at 405 nm.

The activity of PA was calculated as the fold increase in proliferation (as determined by the acid phosphatase activity, OD 405 nm) relative to (1) cell only background, and (2) relative to maximum stimulation by VEGF. VEGF (at 3-10 ng/ml) and FGF (at 1-5 ng/ml) were employed as an activity reference for maximum stimulation. Results of the assay were considered “positive” if the observed stimulation was greater than or equal to a 50% increase over background.

PA assayed “positive” as follows:
1% dilution=fold stimulation
0.1% dilution=fold stimulation
0.01% dilution=fold stimulation

Compared to VEGF (5 ng/ml) control:
1% dilution=fold stimulation

Compared to FGB (5 ng/ml) control:
1% dilution=fold stimulation

### EXAMPLE 12

### Inhibition of Vascular Endothelial Growth Factor (VEGF) Stimulated Proliferation of Endothelial Cell Growth

The ability of various angogenesis inhibitory polypeptides (AIP) to inhibit VEGF stimulated proliferation of endothelial cells was tested. Specifically, bovine adrenal cortical capillary endothelial cells (ACE) (from primary culture, maximum of 12-14 passages) were plated in 96-well plates at 500 cells/well per 100 microliter. Assay media included low glucose DMEM, 10% calf serum, 2 mM glutamine, and 1× penicillin/streptomycin/fungizone. Control wells included the following: (1) no ACE cells added; (2) ACE cells alone; (3) ACE cells plus 5 ng/ml FGF; (4) ACE cells plus 3 ng/ml VEGF; (5) ACE cells plus 3 ng/ml VEGF plus 1 ng/ml TGF-beta; and (6) ACE cells plus 3 ng/ml VEGF plus 5 ng/ml LIF. The test samples, poly-his tagged PRO polypeptides (in 100 microliter volumes), were then added to the wells (at dilutions of 1%, 0.1% and 0.01%, respectively). The cell cultures were incubated for 6-7 days at 37° C./5% CO2. After the incubation, the media in the wells was aspirated, and the cells were washed 1× with PBS. An acid phosphatase reaction mixture (100 microliter; 0.1M sodium acetate, pH 5.5, 0.1% Triton X-100, 10 mM p-nitrophenyl phosphate) was then added to each well. After a 2 hour incubation at 37° C., the reaction was stopped by addition of 10 microliters 1N NaOH. Optical density (OD) was measured on a microplate reader at 405 nm.

The activity of AIP was calculated as the percent inhibition of VEGF (3 ng/ml) stimulated proliferation (as determined by measuring acid phosphatase activity at OD 405 nm) relative to the cells without stimulation. TGF-beta was employed as an activity reference at 1 ng/ml, since TGF-beta blocks 70-90% of VEGF-stimulated ACE cell proliferation. The results, are indicative of the utility of the AIP in cancer therapy and specifically in inhibiting tumor angiogenesis. The numerical values (relative inhibition) are determined by calculating the percent inhibition of VEGF stimulated proliferation by the AIP relative to cells without stimulation and then dividing that percentage into the percent inhibition obtained by TGF-β at 1 ng/ml which is known to block 70-90% of VEGF stimulated cell proliferation. The results are considered positive if the AIP exhibits 30% or greater inhibition of VEGF stimulation of endothelial cell growth (relative inhibition 30% or greater).

### EXAMPLE 13

### Induction of c-fos in Endothelial Cells

This assay is designed to determine whether PA polypeptide or AIP show the ability to induce c-fos in endothelial cells.

Human venous umbilical vein endothelial cells (HUVEC, Cell Systems) in growth media (50% Ham's F12 w/o GHT: low glucose, and 50% DMEM without glycine: with NaHCO3, 1% glutamine, 10 mM HEPES, 10% FBS, 10 ng/ml bFGF) were plated on 96-well microtiter plates at a cell density of 1×104 cells/well. The day after plating, the cells were starved by removing the growth media and treating the cells with 100 μl/well test samples and controls (positive control: growth media; negative control: 10 mM HEPES, 140 mM NaCl, 4% (w/v) mannitol, pH 6.8). The cells were incubated for 30 minutes at 37° C., in 5% CO2. The samples were removed, and the first part of the bDNA kit protocol (Chiron Diagnostics, cat. #6005-037) was followed, where each capitalized reagent/buffer listed below was available from the kit.

Briefly, the amounts of the TM Lysis Buffer and Probes needed for the tests were calculated based on information provided by the manufacturer. The appropriate amounts of thawed Probes were added to the TM Lysis Buffer. The Capture Hybridization Buffer was warmed to room temperature. The bDNA strips were set up in the metal strip holders, and 100 μl of Capture Hybridization Buffer was added to each b-DNA well needed, followed by incubation for at least 30 minutes. The test plates with the cells were removed from the incubator, and the media was gently removed using the vacuum manifold. 100 μl of Lysis Hybridization Buffer with Probes were quickly pipetted into each well of the microtiter plates. The plates were then incubated at 55° C. for 15 minutes. Upon removal from the incubator, the plates were placed on the vortex mixer with the microtiter adapter head and vortexed on the #2 setting for one minute. 80 μl of the lysate was removed and added to the bDNA wells containing the Capture Hybridization Buffer, and pipetted up and down to mix. The plates were incubated at 53° C. for at least 16 hours.

On the next day, the second part of the bDNA kit protocol was followed. Specifically, the plates were removed from the incubator and placed on the bench to cool for 10 minutes. The volumes of additions needed were calculated based upon information provided by the manufacturer. An Amplifier Working Solution was prepared by making a 1:100 dilution of the Amplifier Concentrate (20 fm/μl) in AL Hybridization Buffer. The hybridization mixture was removed from the plates and washed twice with Wash A. 50 μl of Amplifier Working Solution was added to each well and the wells were incubated at 53° C. for 30 minutes. The plates were then removed from the incubator and allowed to cool for 10 minutes. The Label Probe Working Solution was prepared by making a 1:100 dilution of Label Concentrate (40 pmoles/μl) in AL Hybridization Buffer. After the 10-minute cool-down period, the amplifier hybridization mixture was removed and the plates were washed twice with Wash A. 50 μl of Label Probe Working Solution was added to each well and the wells were incubated at 53° C. for 15 minutes. After cooling for 10 minutes, the Substrate was warmed to room temperature. Upon addition of 3 μl of Substrate Enhancer to each ml of Substrate needed for the assay, the plates were allowed to cool for 10 minutes, the label hybridization mixture was removed, and the plates were washed twice with Wash A and three times with Wash D. 50 μl of the Substrate Solution with Enhancer was added to each well. The plates were incubated for 30 minutes at 37° C. and RLU was read in an appropriate luminometer.

The replicates were averaged and the coefficient of variation was determined. The measure of activity of the fold increase over the negative control (HEPES buffer described above) value was indicated by chemiluminescence units (RLU). The results are considered positive if the PRO polypeptide exhibits at least a two-fold value over the negative control. Negative control=1.00 RLU at 1.00% dilution. Positive control=8.39 RLU at 1.00% dilution.

### EXAMPLE 14

### Human Venous Endothelial Cell Ca Flux Assay

This assay is designed to determine whether PA polypeptide or AIP show the ability to stimulate calcium flux in human umbilical vein endothelial cells (HUVEC, Cell Systems). Ca influx is a well-documented response upon binding of certain ligands to their receptors. A test compound that results in a positive response in the present Ca influx assay can be said to bind to a specific receptor and activate a biological signaling pathway in human endothelial cells. This could ultimately lead, for example to cell division, inhibition of cell proliferation, endothelial tube formation, cell migration, apoptosis, etc.

Human venous umbilical vein endothelial cells (HUVEC, Cell Systems) in growth media (50:50 without glycine, 1% glutamine, 10 mM Hepes, 10% FBS, 10 ng/ml Bfgf), were plated on 96-well microtiter ViewPlates-96 (Packard Instrument Company Part #6005182) microtiter plates at a cell density 2×104 cells/well. The day after plating, the cells were washed three times with buffer (HBSS plus 10 mM Hepes), leaving 100 μl/well. Then 100 μl/well of 8 μM Fluo-3 (2×) was added. The cells were incubated for 1.5 hours at 37° C./5% CO2. After incubation, the cells were then washed 3× with buffer (described above) leaving 100 μl/well. Test samples of PA polypeptide or AIP were prepared on different 96-well plates at 5× concentration in buffer. The positive control corresponded to 50 μM ionomycin (5×); the negative control corresponded to Protein 32. Cell plate and sample plates were run on a FLIPR (Molecular Devices) machine. The FLIPR machine added 25 μl of test sample to the cells, and readings were taken every second for one minute, then every 3 seconds for the next three minutes.

The fluorescence change from baseline to the maximum rise of the curve (Δ change) was calculated, and replicates averaged. The rate of fluorescence increase was monitored, and only those samples which had a Δ change greater than 1000 and a rise within 60 seconds, were considered positive.

### EXAMPLE 15

### Induction of Endothelial Cell Apoptosis

The ability of PA polypeptide or AIP to induce apoptosis in endothelial cells was tested in human venous umbilical vein endothelial cells (HUVEC, Cell Systems), using a 96-well format, in 0% serum media supplemented with 100 ng/ml VEGF. (As HUVEC cells are easily dislodged from the plating surface, all pipetting in the wells must be done as gently as practicable.)

The medium was aspired and the cells washed once with PBS. 5 ml of 1× trypsin was added to the cells in a T-175 flask, and the cells were allowed to stand until they were released from the plate (about 5-10 minutes). Trypsinization was stopped by adding 5 ml of growth media. The cells were spun at 1000 rpm for 5 minutes at 4 C. The media was aspirated and the cells were resuspended in 10 ml of 10% serum complemented medium (Cell Systems), 1× penicillin/streptomycin.

The cells were plated on 96-well microtiter plates (Amersham Life Science, cytostar-T scintillating microplate, RPNQ160, sterile, tissue-culture treated, individually wrapped), in 10% serum (CSG-medium, Cell Systems), at a density of 2×104 cells per well in a total volume of 100 μl. PA polypeptide was added in triplicate at dilutions of 1%, 0.33% and 0.11%. Wells without cells were used as a blank and wells with cells only were used as a negative control. As a positive control 1:3 serial dilutions of 50 μl of a 3× stock of staurosporine were used. The ability of the PA polypeptide or AIP to induce apoptosis was determined using Annexin V, a member of the calcium and phospholipid binding proteins, to detect apoptosis.

0.2 ml Annexin V—Biotin stock solution (100 μg/ml) were diluted in 4.6 ml 2× Ca2+ binding buffer and 2.5% BSA (1:25 dilution). 50 μls of the diluted Annexin V—Biotin solution were added to each well (except controls) to a final concentration of 1.0 μg/ml. The samples were incubated for 10-15 minutes with Annexin-Biotin prior to direct addition of 35S-Streptavidin. 35S-Streptavidin was diluted in 2× Ca2+ Binding buffer, 2.5% BSA and was added to all wells at a final concentration of 3×104 cpm/well. The plates were then sealed, centrifuged at 1000 rpm for 15 minutes and placed on orbital shaker for 2 hours. The analysis was performed on 1450 Microbeta Trilux (Wallac).

### EXAMPLE 16

### Enhancement of Heart Neonatal Hypertrophy

This assay is designed to measure the ability of a PA polypeptide to stimulate hypertrophy of neonatal heart.

Cardiac myocytes from 1-day old Harlan Sprague Dawley rats were obtained. Cells (180 μl at 7.5×104/ml, serum <0.1%, freshly isolated) are added on day 1 to 96-well plates previously coated with DMEM/F12+4% FCS. Test samples containing the test PA polypeptide or growth medium only (negative control) (20 μl/well) are added directly to the wells on day 1. PGF (20 μl/well) is then added on day 2 at final concentration of 10-6 M. The cells are then stained on day 4 and visually scored on day 5, wherein cells showing no increase in size as compared to negative controls are scored 0.0, cells showing a small to moderate increase in size as compared to negative controls are scored 1.0 and cells showing a large increase in size as compared to negative controls are scored 2.0.

### EXAMPLE 17

### Induction of c-fos in Endothelial Cells

This assay is designed to determine whether a PA polypeptide shows the ability to induce c-fos in endothelial cells.

Human venous umbilical vein endothelial cells (HUVEC, Cell Systems) in growth media (50% Ham's F12 w/o GHT: low glucose, and 50% DMEM without glycine: with NaHCO3, 1% glutamine, 10 mM HEPES, 10% FBS, 10 ng/ml bFGF) were plated on 96-well microtiter plates at a cell density of 1×104 cells/well. The day after plating, the cells were starved by removing the growth media and treating the cells with 100 μl/well test samples and controls (positive control: growth media; negative control: 10 mM HEPES, 140 mM NaCl, 4% (w/v) mannitol, pH 6.8). The cells were incubated for 30 minutes at 37° C., in 5% CO2. The samples were removed, and the first part of the bDNA kit protocol (Chiron Diagnostics, cat. #6005-037) was followed, where each capitalized reagent/buffer listed below was available from the kit.

Briefly, the amounts of the TM Lysis Buffer and Probes needed for the tests were calculated based on information provided by the manufacturer. The appropriate amounts of thawed Probes were added to the TM Lysis Buffer. The Capture Hybridization Buffer was warmed to room temperature. The bDNA strips were set up in the metal strip holders, and 100 μl of Capture Hybridization Buffer was added to each b-DNA well needed, followed by incubation for at least 30 minutes. The test plates with the cells were removed from the incubator, and the media was gently removed using the vacuum manifold. 100 μl of Lysis Hybridization Buffer with Probes were quickly pipetted into each well of the microtiter plates. The plates were then incubated at 55° C. for 15 minutes. Upon removal from the incubator, the plates were placed on the vortex mixer with the microtiter adapter head and vortexed on the #2 setting for one minute. 80 μl of the lysate was removed and added to the bDNA wells containing the Capture Hybridization Buffer, and pipetted up and down to mix. The plates were incubated at 53° C. for at least 16 hours.

On the next day, the second part of the bDNA kit protocol was followed. Specifically, the plates were removed from the incubator and placed on the bench to cool for 10 minutes. The volumes of additions needed were calculated based upon information provided by the manufacturer. An Amplifier Working Solution was prepared by making a 1:100 dilution of the Amplifier Concentrate (20 fm/μl) in AL Hybridization Buffer. The hybridization mixture was removed from the plates and washed twice with Wash A. 50 μl of Amplifier Working Solution was added to each well and the wells were incubated at 53° C. for 30 minutes. The plates were then removed from the incubator and allowed to cool for 10 minutes. The Label Probe Working Solution was prepared by making a 1:100 dilution of Label Concentrate (40 pmoles/μl) in AL Hybridization Buffer. After the 10-minute cool-down period, the amplifier hybridization mixture was removed and the plates were washed twice with Wash A. 50 μl of Label Probe Working Solution was added to each well and the wells were incubated at 53° C. for 15 minutes. After cooling for 10 minutes, the Substrate was warmed to room temperature. Upon addition of 3 μl of Substrate Enhancer to each ml of Substrate needed for the assay, the plates were allowed to cool for 10 minutes, the label hybridization mixture was removed, and the plates were washed twice with Wash A and three times with Wash D. 50 μl of the Substrate Solution with Enhancer was added to each well. The plates were incubated for 30 minutes at 37° C. and RLU was read in an appropriate luminometer.

The replicates were averaged and the coefficient of variation was determined. The measure of activity of the fold increase over the negative control (HEPES buffer described above) value was indicated by chemiluminescence units (RLU). Samples that show an at least two-fold value over the negative control value were considered positive.

PA polypeptide assayed “positive” two times:
(1) Negative Control=RLU
   Positive control=RLU
   PA at 0.01%=RLU
(2) Negative control=RLU
   Positive control=RLU
   PA at 0.01%=RLU

### EXAMPLE 18

### Guinea Pig Vascular Leak

This assay is designed to determine whether PA polypeptide shows the ability to induce vascular permeability.

Hairless guinea pigs weighing 350 grams or more were anesthetized with Ketamine (75-80 mg/kg) and 5 mg/kg Xylazine intramuscularly. Test samples containing the PA polypeptide or a physiological buffer without the test polypeptide are injected into skin on the back of the test animals with 100 μl per injection site intradermally. There were approximately 16-24 injection sites per animal. One ml of Evans blue dye (1% in PBS) is then injected intracardially. Skin vascular permeability responses to the compounds (i.e., blemishes at the injection sites of injection) are visually scored by measuring the diameter (in mm) of blue-colored leaks from the site of injection at 1 and 6 hours post administration of the test materials. The mm diameter of blueness at the site of injection is observed and recorded as well as the severity of the vascular leakage. Blemishes of at least 5 mm in diameter are considered positive for the assay when testing purified proteins, being indicative of the ability to induce vascular leakage or permeability. A response greater than 7 mm diameter is considered positive for conditioned media samples. Human VEGF at 0.1 μg/100 μl is used as a positive control, inducing a response of 15-23 mm diameter.

### EXAMPLE 19

### Taqman™ PCR Analysis of the Time Dependence of Expression of Angiogenic Associated Genes

Validation and Confirmation of Gene Expression by Quantitative RT-PCR (Taqman™). To confirm the expression data from GeneCalling™ by an independent technique, HUVECs were grown within collagen gels as the model system that promotes vascular tube formation. Gene specific PCR oligonucleotide primer pairs and oligonucleotide probes labeled with a reporter fluorescent dye at the 5′ end and quencher fluorescent dye at the 3′ end were designed using the Oligo 4.0 software (National Bioscience, Plymouth Minn.). Total RNA (50 ng) obtained from the resulting cells at various time points ranging from 30 min to almost 2 days was added to a 50 ml RT-PCR reaction mixture according to the manufacturer's protocol (Roche Molecular Systems Inc. Branchburg, N.J.). The thermal cycling conditions included 1 cycle at 48° C. for 30 min, 1 cycle at 95° C. for 10 min, 40 cycles at 95° C. for 15 s, annealing at 60° C. for 1 min, and a final hold at 25° for 2 min. Standard curves for the expression of each gene were generated by serial dilution of a standard preparation of total RNA isolated from quiescent HUVEC grown in monolayer culture. Data are expressed as the fold induction normalized to the same gene from quiescent HUVEC RNA. For comparison, data obtained with RNA extracted from HUVECs grown on collagen film, on which differentiation into tube-like structures does not occur, is included as a control.

Table 4 shows the TaqMan™ primers and probe sets used.

TABLE 4
Taqman Primer and Probes Sets

| | | | | |
|---|---|---|---|---|
| Gene Name | PA # | Forward Primer | Reverse Primer | Probe |

Hormones/Growth
Factors

| | | | | |
|---|---|---|---|---|
| Placental Growth Factor | 22 | GACGTTCTCTCAGCACGTTCG | CACCTTTCCGGCTTCATCTTC | CGAATGCCGGCCTCTGCGG |
| (PlGF) | | (SEQ ID NO:3) | (SEQ ID NO:4) | (SEQ ID NO:5) |
| Stanniocalcin Precursor | 23 | CGAGTGGCGGCTCAAAA | CCGCAGCCGACCTGTAGA | TCAGCTGAAGTGGTTCGTTGCCTCAA |
| | | (SEQ ID NO:6) | (SEQ ID NO:7) | (SEQ ID NO:8) |
| Fibroblast Growth Factor | 24 | CCTTAGCTGACTCCCCAGGTT | CTGCAGCTTCCCCTCGATT | CCTGAACGAGCGCCTGGGCC |
| 16 (FGF-16) | | (SEQ ID NO:9) | (SEQ ID NO:10) | (SEQ ID NO:11) |

Tyrosine Kinase
Receptors

| | | | | |
|---|---|---|---|---|
| ax1 | 15 | GCATGAAGGAATTTGACCAT | TCTCTCGTTCAGAACCCTGGA | CAGACACCGATGAGCCTCATGACGTT |
| | | CC | (SEQ ID NO:13) | (SEQ ID NO:14) |
| | | (SEQ ID NO:12) | | |
| Epithelial Cell Tyrosine | 16 | GCCTGTTCACCAAGATTGACA | GCCTCGAAGTCGCTGCTG | TTGCGCCCGATGAGATCACCG |
| Kinase (ECK) | | C | Q | (SEQ ID NO:17) |
| | | (SEQ ID NO:15) | (SEQ ID NO:16) | |

Other Receptors/Integral Membrane Glycoproteins

| | | | | |
|---|---|---|---|---|
| OX40 | 17 | CCAACTCTGCACCGTTCTAGG | GGTATGCATGGCATACGTAA | CCGATGGCTGCCTCCGGCT |
| | | (SEQ ID NO:18) | GC | (SEQ ID NO:20) |
| | | | (SEQ ID NO:19) | |
| Podocalyxin-like Protein | 3 | GGGCATGGTGAGGTTTCATCT | TTTACGCCCAGAACGATGG | CCATGGCGAAAGTTCAACATTCCACA |
| | | (SEQ ID NO:21) | (SEQ ID NO:22) | (SEQ ID NO:23) |
| Alpha-2 integrin | 21 | TCTGAGACTGCCAAGGTCTTC | CAGCTGGTATTTGTCGGACAT | AGGACTAGATCAGAAATGCAAAGTCC |
| | | A | C | ATCCTCA |
| | | (SEQ ID NO:24) | (SEQ ID NO:25) | (SEQ ID NO:26) |
| Gp130 | 18 | ATCCGCGCAAGATGTTGAC | ACCTGTAGATTCAGTGGTGAG | ACAAGGCTTGCACTACCCAAGTCTGCA |
| | | (SEQ ID NO:27) | GAAA | (SEQ ID NO:29) |
| | | | (SEQ ID NO:28) | |
| Protein zero related | 20 | TGTGTCATATCAATTTCTGGA | TTGATCCAACTGTGTCCAGAA | TGACTTCGGCATTTATCCTTTGCTAAT |
| protein | | TTCATAA | TG | CTTGCT |
| | | (SEQ ID NO:30) | (SEQ ID NO:31) | (SEQ ID NO:32) |
| CD82 | 19 | CGACACGTGGGCACAGG | AGCTTCCTTCCACGAAACCA | CAGCTGGTCACAGGGCCCACTTCT |
| | | (SEQ ID NO:33) | (SEQ ID NO:34) | (SEQ ID NO:35) |

Proteases/Protease
Inhibitors

| | | | | |
|---|---|---|---|---|
| Tissue Factor Pathway | 13 | CGATGCTTGCTGGAGGATAG | ACACTGGTCGTCCACACTCAC | AAAGTTCCCAAAGTTTGCCGGCTGC |
| Inhibitor-2 (TFPI-2) | | A | T | (SEQ ID NO:38) |
| | | (SEQ ID NO:36) | (SEQ ID NO:37) | |
| Aggrecanase (ADAMTS4; | 12 | ACTGGTGGTGGCAGATGACA | TCACTGTTAGCAGGTAGCGCT | ATGGCCGCATTCCACGGTGC |
| KIAA0688) | | (SEQ ID NO:39) | TT | (SEQ ID NO:41) |
| | | | (SEQ ID NO:40) | |
| Cathepsin B | 11 | GAAGCCATCTCTGACCGGATC | TCCGCCGACACCTCCA | CCACACCAATGCGCACGTCAGC |
| | | (SEQ ID NO:42) | (SEQ ID NO:43) | (SEQ ID NO:44) |
| Plasminogen Activator | 14 | GCAGGCACAAGCTGCAGATA | CCTGTGGATGCATTGATTGC | TCCATTCATCCTTCCGCTCTCTCAGC |
| Inhibitor-2 (PAI-2) | | (SEQ ID NO:45) | (SEQ ID NO:46) | (SEQ ID NO:47) |
| Transporter/Channels | | | | |
| White Protein Homolog | 25 | CCCTTTCAGATCATGTTCCCA | GGACGGCTGCGACGTC | CCAGTACACGATGCTGCAGTAGGCCA |
| | | (SEQ ID NO:48) | (SEQ ID NO:49) | (SEQ ID NO:50) |

Cytoskeleton/Motility

| | | | | |
|---|---|---|---|---|
| Moesin | 4 | ACTGGGCCGAGACAAATACA | AATGCGCTGCTTGGTGTTG | CCCTGCGCCAGATCCGGC |
| | | A | (SEQ ID NO:52) | (SEQ ID NO:53) |
| | | (SEQ ID NO:51)) | | |
| actin bundling protein | 8 | CCAGCTGCTACTTTGACATCG | CCATTGGACGCCCTCAGT | GATGCGCCGGTCACGCCA |
| | | A | (SEQ ID NO:55) | (SEQ ID NO:56) |
| | | (SEQ ID NO:54) | | |
| T-plastin | 7 | AATAAAACAGCCATGCTCCC | CCTTAAGCCATAAGCACTTCA | TGCATGATTCGCAGGTCAGCTATTTCC |
| | | A | CC | (SEQ ID NO:59) |
| | | (SEQ ID NO:57) | (SEQ ID NO:58) | |
| brain ankyrin-2 | 10 | AAGCAGCTTCCTGATGCATTC | CGGACACAGCGCCTTACAT | TCGCAGCCAAGAACAGCCACCA |
| | | (SEQ ID NO:60) | (SEQ ID NO:61) | (SEQ ID NO:62) |
| Intermediate Filaments | | | | |
| Mesothelial Keratin K7 | 5 | CCCAGATCTCCGACACATCTG | GCGATGATGCCGTCCAG | CCATGGACAACAGTCGCTCCCTGG |
| | | (SEQ ID NO:63) | (SEQ ID NO:64) | (SEQ ID NO:65) |

Extracellular Matrix

| | | | | |
|---|---|---|---|---|
| Laminin gamma 2 (Nicein | 2 | GCTGACAGGCAGGTGTTTGA | CGAAGTAGCCTGCTTTGCACT | TGTATCCACAACACAGCCGGCATCTAC |
| B2 Chain) | | A | (SEQ ID NO:67) | TG |
| | | (SEQ ID NO:66) | | (SEQ ID NO:68) |
| Nidogen-2 (osteonidogen) | 1 | AAAATCTTAGAACTTTTGTTG | CCTTGACAGTTGGAGAAGCC | AAATAATTGGTCCTTTCCCATCAGTTC |
| | | GGAAACTA | A | TGCA |
| | | (SEQ ID NO:69) | (SEQ ID NO:70) | (SEQ ID NO:71) |

### EXAMPLE 20

### In situ Hybridization of Three PA Genes

Fluorescence in situ hybridization (FISH) in the vasculature associated with tumors and with inflammatory disease for three PAP genes identified from the differential expression analysis disclosed herein was carried out by the method described in Rosen, B. et al., Trends Genet. 9:162-167 (1993). Samples were preserved and prepared for microscopic examination. Then, either the same sections were simultaneously treated with haematoxylin-eosin and fluorescent in situ hybridization probes, or adjacent sections were treated respectively with one or the other preparation. These results are shown in FIGS. 26-29.

Results for podocalyxin [PA3] mRNA expression are shown in FIG. 26. Podocalyxin-like protein shows moderate to strong expression in vessels surrounding lung squamous cell carcinoma (arrows). In addition, it is expressed in nearly all small arterioles, and in a subset of small veins and capillaries in adrenal cortex and skeletal muscle in fetal tissues. In adult tissues expression is limited to podocytes, some endothelial cells in the adventitia around large vessels, in the outer nuclear cell layer and inner segment of the photoreceptor layer of the retina. In tumor tissues, expression is moderate to weak in the endothelium of small vessels (usually arterioles rather than venules or capillaries) associated with chondrosarcoma, squamous cell carcinoma of the oral mucosa, squamous cell carcinoma of the lung, ductal mammary adenocarcinoma, and renal cell carcinoma. Expression was observed in the arteriolar endothelium in inflamed adipose tissue from a sample of appendicitis. These data, and the real-time quantititative expression analysis of podocalyxin like protein in an in vitro vascular morphogenesis assay support the new uses described in Table 2 and the claimed methods of the invention.

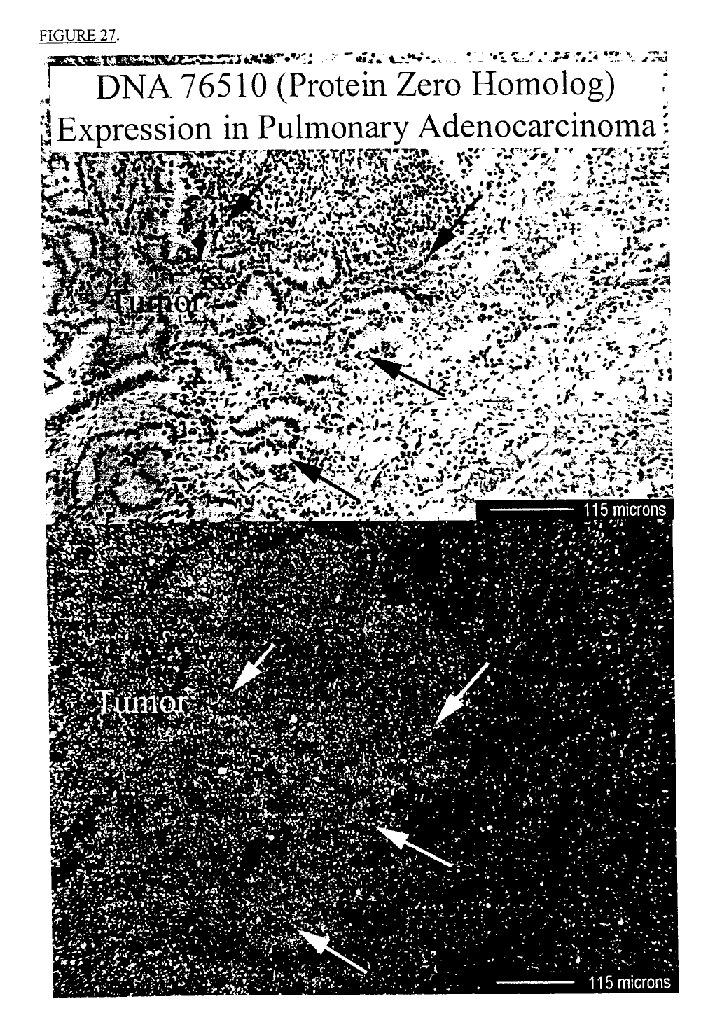

FIG. 27 shows the results for a protein zero (PZR) [PA21] probe. It is seen that PZR is expressed in elevated levels in malignant epithelium from pulmonary adenocarcinoma as compared to normal epithelium. Similar results are obtained in renal cell carcinoma and mammary ductal adenocarcinoma. In general, higher levels of PZR are found in actively replicating cell populations (many fetal tissues, basal epithelial layers, and tumor cells). In situ hybridization did not reflect a specific bias for vascular cell types, though the real time quantitative PCR results clearly indicate a modulation of PZR in this endothelial cell tube formation system. The discrepancy can easily be accounted for by the fact that the tube formation is a transient intermediate during vascular morphogenesis (See Yang, et al., Am J Pathol Septmeber 1999; 155(3):887-95).

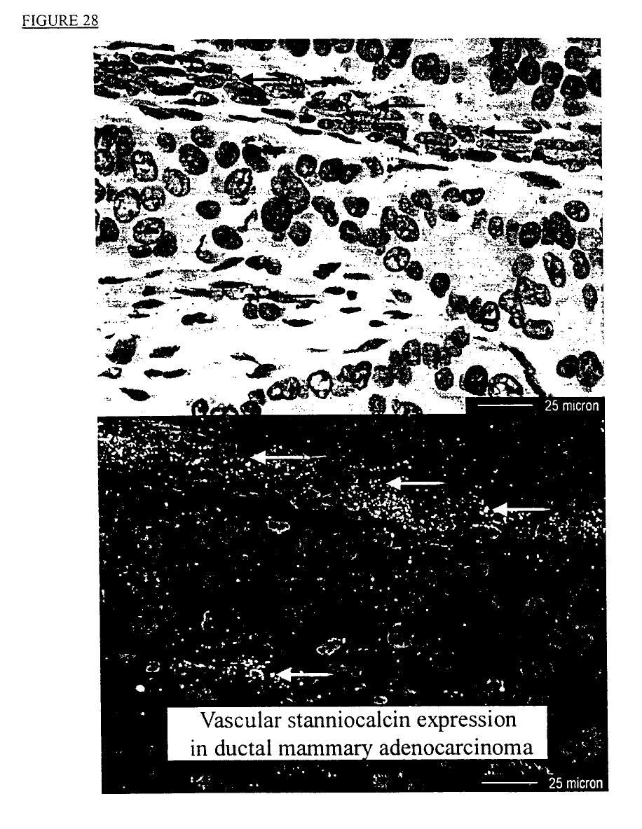

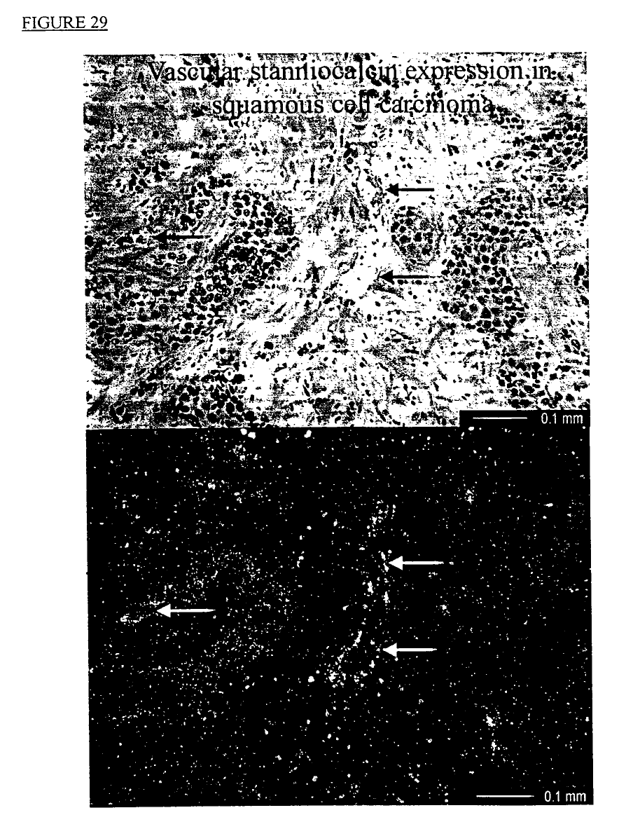

FIGS. 28 and 29 show haematoxylin-eosin staining and in situ hybridization carried out for expression of stanniocalcin precursor [PA23] mRNA in ductal mammary adenocarcinoma and in squamous cell carcinoma, respectively. It is seen that the stanniocalcin probe shows strong, but variable, expression in and around the carcinoma tissue. It is also found to a lesser extent in chondrosarcoma and renal cell carcinoma vasculature. Some expression is also observed in small vessels in first trimester placental villi. It is important to the specificity of this molecule as an indicator of angiogenic morphogenesis that stanniocalcin is not significantly expressed elsewhere in the vascular system. A further important observation is that no significant expression is seen in normal vessels.

### EXAMPLE 21

### Novel Gene Differentially Expressed in Endothelial Tube Formation

The QEA method employed herein provided a gene fragment designated r0v0-176.7 [PA27]. This was matched 100% to a human sequence not present in any public database available to the inventors. This assembly, termed r0v0-176.7A, is 99% to similar to AF173937, a secreted protein of unknown function, which may be only a partial sequence. In addition, r0v0-176.7A has 47 of 95 residues identical to and 68 of 95 residues positive with a 100 residue putative Arabidopsis thaliana steroid binding protein (TREMBLNEW-ACC:AAD23019); and has 52 of 140 residues identical to, and 73 of 140 residues positive with a 194 residue rat membrane associated progesterone receptor protein (SWISSNEW-ACC:P70580). The amino acid sequence of PA27 is included in FIG. 30. TaqMan™ anlysis performed as described in Example 19 reveals that its expression peaks at 4 h, and that the ratio between gel/film is 0.5 early, 1.0 at 4 h, 1.5 at 8 h and then decreases to 0.25 at 46.5 h.

Testosterone and dexamethasone are strong inhibitors and all-trans retinoic acid (at-RA) and 9-cis retinoic acid (9-cis RA) are potent stimulators of the formation of capillary-like tubular structures (UI: 98345318). Also prostaglandin is an inhibitor. It is therefore possible that endothelial cells release the r0v0-176.7A protein to bind and sequester steroid-like hormones in the process of tube formation.

SignalP and Signal Peptide analyses find a signal peptide with a cleavage site either at residue 27 or residue 32. PSORT predicts that the protein localizes extracellularly. Sbase finds homology to CYTOCHROME B5 AND OXIDOREDUCTASES Heme binding domain between residues 45 to 90, homology to a transmembrane region between residues 96 and 145; and homology to a kinase region between residues 3 and 46.

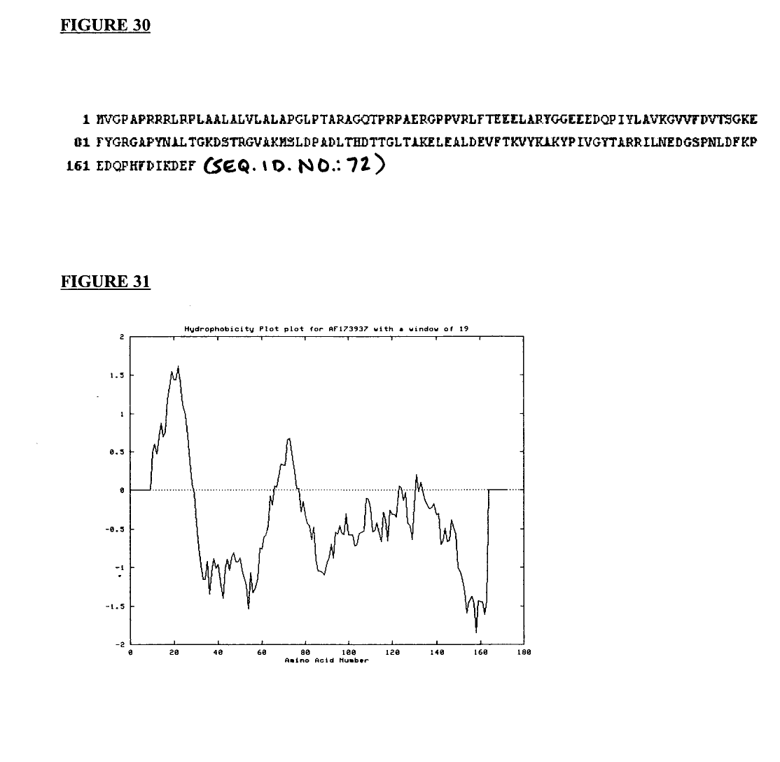

A hydropathy plot of r0v0-176.7A is shown in FIG. 31.

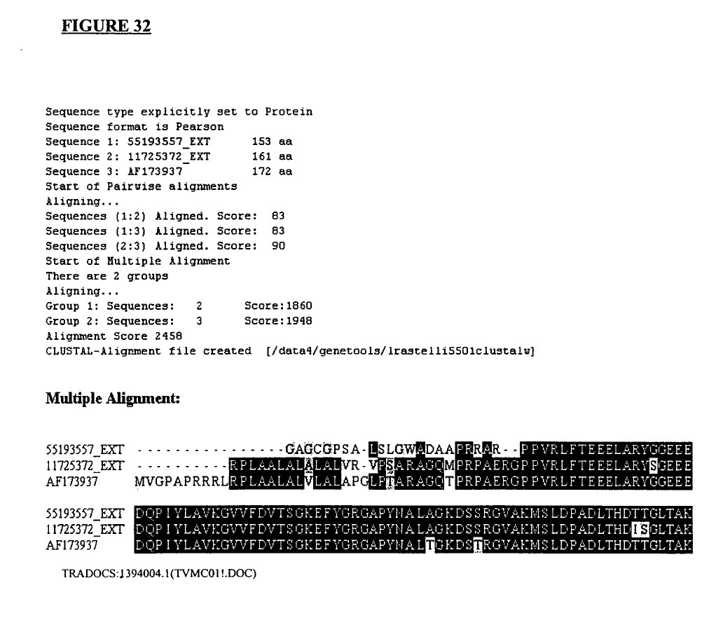

The mouse and rat orthologs of r0v0-176.7A were assembled. The results are shown in a ClustalW alignment in FIG. 32. The rat ortholog is highly similar to the human sequence and the two are highly coextensive. The mouse sequence has a mismatch in the NH region resulting in a frameshift. All three orthologs species arise from numerous ESTs, indicating that they are highly express. Also most of the ESTs are of fetal origin, or from tumors (ovarian cancer). This is further evidence of a possible role in angiogenesis.

The r0v0-176.7A genes and the polypeptides they encode are believed to be essential components, singly or severally, in the biologic pathway(s) of endothelial cell tube formation or anglogenesis.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1.-8. (canceled)

9. A method of diagnosing an angiogenic disorder in a subject, the method comprising:
a) providing from the subject a test cell population comprising cells capable of expressing one or more nucleic acid sequences selected from the group consisting of PA:1-27;
b) detecting expression of one or more of the nucleic acid sequences in said test cell population;
c) comparing the expression of the nucleic acid sequences in the test cell population to the expression of the nucleic acid sequences in a reference cell population comprising at least one cell whose angiogenic stage is known; and
d) identifying a difference in expression levels of the PA:1-27 sequences, if present, in the test cell population and the reference cell population,
thereby diagnosing an angiogenic disorder in the subject.

10. (canceled)

11. The method of claim 9, wherein the subject is human.

12. The method of claim 9, wherein the expression of the nucleic acid sequences in the test cell population is increased as compared to the reference cell population.

13. The method of claim 9, wherein the expression of the nucleic acid sequences in the test cell population is decreased as compared to the reference cell population.

14. The method of claim 9, wherein the test cell population is provided in vitro.

15. The method of claim 9, wherein the test cell population is provided ex vivo from a mammalian subject.

16.-68. (canceled)

69. A method of diagnosing an angiogenic disorder in a subject comprising:
a) determining the presence or amount of expression of stanniocalcin in a sample obtained from the subjet as compared to a control sample; and
b) determing if there is a difference in the presence or amount of expression of stanniocalcin in the sample as compared to a control sample, wherein the difference in presence or amount of expression is indicative of the angiogenic disorder in the subject.

70. The method of claim 69, wherein the sample is selected from the group consisting of blood, tumor tissue, cells, and serum.

71. The method of claim 69, wherein determing the presence or amount of expression of stanniocalcin comprises detecting the presence or amount of a nucleic acid encoding stanniocalcin.

72. The method of claim 71, wherein the detecting the presence or amount of the daily nucleic acid encoding stanniocalcin comprises hybridizing the sample to a probe that specifically binds to a nucleic acid encoding stanniocalcin, or amplifying nucleic acids in the sample using primers that specifically bind to the nucleic acid encoding stanniocalcin.

73. The method of claim 71, wherein detecting the presence or amount of expression of stanniocalcin comprises detecting the presence or amount of stanniocalcin in the sample with an antibody that speciically bunds to stanniocalcin.

74. The method of claim 69, wherein the difference in the presence or amunt of expression of stanniocalcin in the sample is an increase in the presence or amount of expression of stanniocalcin and is indicative of an angiogenic disorder having an increase in angiogensis.

75. The method of claim 74, wherein the angiogenic disorder having an increase in angiogenesis is cancer or macular degeneration.

76. The method of claim 75, wherein the cancer is selected from the group consisting of breast, colon, ovarian, liver, kidney and prostate cancer.

77. The method of claim 69. wherein the difference in the presence or amount of expression of stanniocalcin in the sample is a decrease in the presence or amount of expression of stannioclcin and is indicative of a disorder have a decrease in angiogensis.

78. A method of diagnosing an angiogenic disorder, wherein the angio genic disorder is metastatic cancer in a subject comprising:
a) determing the presence or amount of expression of stanniocalcin in a sample obtained from the subject as compared to a control sample; and
b) determing if ther is a difference in the presence or amount of expression stanniocalcin in the sample as compared to a control sample, wherein an increase on the presence or amount of expression of stanniocalcin in the sample is indicative of metastatic cancer in the subject.

79. The method claim 8, wherein the sample is selected from the group consisting of blood, tumor tissue, cells, and serum.

80. The methid of claim 78 wherein determining the process or amount of expression of stanniocalcin comprises detecting the presence or amount of a nucleic acid encoding stanniocalcin.


SEQUENCE LISTING (76 sequences)

SEQ ID NO: 1 - DNA, 1434 bp, Sclerotinia sclerotiorum
  modified_base (473): a, t, c, g, other or unknown
ggccgcgttttcctggggaagcggcgggcggggtggagcagccagctgggtccggggagcgccgccgccgcctcgatggggtgttgaaaagtctcctctagagctttggaaggctgaatgcactaaacatgaagagcttgaaagcgaagttcaggaagagtgacaccaatgagtggaacaagaatgatgaccggctactgcaggccgtggagaatggagatgcggagaaggtggcctcactgctcggcaagaaggggaccagtgccaccaaacacgacagtgagggcaagaccgctttccatcttgctgctgcaaaaggacacgtggaatgcctcagggtcatgattacacatggtgtggatgtgacagcccaagatactaccggacacagcgccttacatctcgcagccaagaacagccaccatgaatgcatcaggaagctgcttcagtctaaatgcccagccgaaagtgtngacagctctgggaaaacagctttacattatgcagcggctcagggctgccttcaagctgtgcagattctctgcgaacacaagagccccataaacctcaaagatttggatgggaatataccgctgctncttgctgtacaaaatggtcacagtgagatctgtcactttctcctggatcatggagcagatgtcaattccaggaacaaaagtggaagaactgctctcatgctggcctgtgagattggcagctctaacgctgtggaagccttaattaaaaagggtgcagacctaaaccttgtagattctcttggatacaatgccttacattattccaaactctcagaaaatgcaggaattcaaagccttctattatcaaaaatctctcaggatgctgatttaaagaccccaacaaaaccaaagcagcatgaccaagtctctaaaataagctcagaaagaagtggaactccaaaaaaacgcaaagctccaccacctcctatcagtcctacccagttgagtgatgtctcttccccaagatcaataacttcgactccactatcgggaaaggaatcggtattttttgctgaaccacccttcaaggctgagatcagttctatacgagaaaacaaagacagactaagtgacagtactacaggtgctgatagcttattggatataagttctgaagctgaccaacaagatcttctctctctattgcaagcaaaagttgcttcccttaccttacacaataaggagttacaagataaattacaggccaaatcacccaaggaggcggaagcagacctaagctttgactcataccattccacccaaactgacttgggcccatccctggggaaaacctggtgaaacctctcccccagactccaaatcatctccatctgtcttaatacattctttaggtaaatccactactggcaatgatgtcagaattcagncaactggc

SEQ ID NO: 2 - DNA, 1458 bp, Homo sapiens
  modified_base (72): a, t, c, g, other or unknown
tctagatcctattggcaaagccacacttgtgaaatctaccccggtcaatgtacccatcagtcagaaatttantgatctgtttgagaagatggttcccgtgtcagtacagcagtctttggctgcctataatcagaggaaagccgatttggttaacagatcaattgctcagatgagagaagccaccactttggcaaatggggtgctagcttcccttaatcttccagcagcaattgaagatgtgtctggagacactgtacctcagtctatattgactaaatccagatctgtgattgaacagggaggcatccagactgttgatcagttgattaaagaactgcctgaattactgcaacgaaatagagaaatcctagatgagtcattaaggttgttggatgaagaagaagcaaccgataatgatttaagagcaaaatttaaggaacgttggcaaaggacaccatccaatgaactgtataagcctttaagagcagagggaaccaacttcagaacagttttagataaagctgtgcaggcagatggacaagtgaaagaatgttaccagtctcatcgtgacaccatcgtgcttttgtgtaagccagagcctgagctgaatgctgccatcccttctgctaatccagcaaagaccatgcagggcagtgaggttgtaaatgtcttaaaatccttattgtcaaatcttgatgaagtaaagaaggaaagagagggtctggagaatgacttgaaatctgtgaattttgacatgacaagcaagtttttgacagccctggctcaagatggtgtgataaatgaagaagctctttctgttactgaactagatcgagtctatggaggtcttacaactaaagtccaagaatctctaaagaaacaggagggacttcttaaaaatattcaggtctcacatcaggaattttcgaaaatgaaacaatctaataatgaagctaacttaagagaagaagttttgaagaatttagctactgcatatgacaactttgttgaacttgtagctaatttgaaggaaggcacaaagttttacaatgagttgactgaaatcctggtcaggttccagaacaaatgcagtgatatagtttttgcacggaagacagaaagagatgaactcttaaaggacttgcaacaaagcattgccagagaacctagtgctccttcaattcctacacctgcgtatcagtcctcaccagcaggaggacatgcaccaactcctccaactccagcgccaagaaccatgccgcctactaagccccagcccccagccaggcctccaccacctgtgcttccagcaaatcgagctccttctgctactgctccatctccagtgggggctgggactgctgcgccagttccatcaacaaacgcctggctcagctcctcctccacaggcgcagggaccaccctatcccacctatccaggatatcctgggtattgcc

SEQ ID NO: 3 - DNA, 21 bp, Artificial Sequence
  Description of Artificial Sequence Primer
gacgttctctcagcacgttcg

SEQ ID NO: 4 - DNA, 21 bp, Artificial Sequence
  Description of Artificial Sequence Primer
cacctttccggcttcatcttc

SEQ ID NO: 5 - DNA, 19 bp, Artificial Sequence
  Description of Artificial Sequence Probe
cgaatgccggcctctgcgg

SEQ ID NO: 6 - DNA, 17 bp, Artificial Sequence
  Description of Artificial Sequence Primer
cgagtggcggctcaaaa

SEQ ID NO: 7 - DNA, 18 bp, Artificial Sequence
  Description of Artificial Sequence Primer
ccgcagccgacctgtaga

SEQ ID NO: 8 - DNA, 26 bp, Artificial Sequence
  Description of Artificial Sequence Probe
tcagctgaagtggttcgttgcctcaa

SEQ ID NO: 9 - DNA, 21 bp, Artificial Sequence
  Description of Artificial Sequence Primer
ccttagctgactccccaggtt

SEQ ID NO: 10 - DNA, 19 bp, Artificial Sequence
  Description of Artificial Sequence Primer
ctgcagcttcccctcgatt

SEQ ID NO: 11 - DNA, 20 bp, Artificial Sequence
  Description of Artificial Sequence Probe
cctgaacgagcgcctgggcc

SEQ ID NO: 12 - DNA, 22 bp, Artificial Sequence
  Description of Artificial Sequence Primer
gcatgaaggaatttgaccatcc

SEQ ID NO: 13 - DNA, 21 bp, Artificial Sequence
  Description of Artificial Sequence Primer
tctctcgttcagaaccctgga

SEQ ID NO: 14 - DNA, 26 bp, Artificial Sequence
  Description of Artificial Sequence Probe
cagacaccgatgagcctcatgacgtt

SEQ ID NO: 15 - DNA, 22 bp, Artificial Sequence
  Description of Artificial Sequence Primer
gcctgttcaccaagattgacac

SEQ ID NO: 16 - DNA, 18 bp, Artificial Sequence
  Description of Artificial Sequence Primer
gcctcgaagtcgctgctg

SEQ ID NO: 17 - DNA, 21 bp, Artificial Sequence
  Description of Artificial Sequence Probe
ttgcgcccgatgagatcaccg

SEQ ID NO: 18 - DNA, 21 bp, Artificial Sequence
  Description of Artificial Sequence Primer
ccaactctgcaccgttctagg

SEQ ID NO: 19 - DNA, 22 bp, Artificial Sequence
  Description of Artificial Sequence Primer
ggtatgcatggcatacgtaagc

SEQ ID NO: 20 - DNA, 19 bp, Artificial Sequence
  Description of Artificial Sequence Probe
ccgatggctgcctccggct

SEQ ID NO: 21 - DNA, 21 bp, Artificial Sequence
  Description of Artificial Sequence Primer
gggcatggtgaggtttcatct

SEQ ID NO: 22 - DNA, 19 bp, Artificial Sequence
  Description of Artificial Sequence Primer
tttacgcccagaacgatgg

SEQ ID NO: 23 - DNA, 26 bp, Artificial Sequence
  Description of Artificial Sequence Probe
ccatggcgaaagttcaacattccaca

SEQ ID NO: 24 - DNA, 22 bp, Artificial Sequence
  Description of Artificial Sequence Primer
tctgagactgccaaggtcttca

SEQ ID NO: 25 - DNA, 22 bp, Artificial Sequence
  Description of Artificial Sequence Primer
cagctggtatttgtcggacatc

SEQ ID NO: 26 - DNA, 33 bp, Artificial Sequence
  Description of Artificial Sequence Probe
aggactagatcagaaatgcaaagtccatcctca

SEQ ID NO: 27 - DNA, 19 bp, Artificial Sequence
  Description of Artificial Sequence Primer
atccgcgcaagatgttgac

SEQ ID NO: 28 - DNA, 25 bp, Artificial Sequence
  Description of Artificial Sequence Primer
acctgtagattcagtggtgaggaaa

SEQ ID NO: 29 - DNA, 27 bp, Artificial Sequence
  Description of Artificial Sequence Probe
acaaggcttgcactacccaagtctgca

SEQ ID NO: 30 - DNA, 28 bp, Artificial Sequence
  Description of Artificial Sequence Primer
tgtgtcatatcaatttctggattcataa

SEQ ID NO: 31 - DNA, 23 bp, Artificial Sequence
  Description of Artificial Sequence Primer
ttgatccaactgtgtccagaatg

SEQ ID NO: 32 - DNA, 33 bp, Artificial Sequence
  Description of Artificial Sequence Probe
tgacttcggcatttatcctttgctaatcttgct

SEQ ID NO: 33 - DNA, 17 bp, Artificial Sequence
  Description of Artificial Sequence Primer
cgacacgtgggcacagg

SEQ ID NO: 34 - DNA, 20 bp, Artificial Sequence
  Description of Artificial Sequence Primer
agcttccttccacgaaacca

SEQ ID NO: 35 - DNA, 24 bp, Artificial Sequence
  Description of Artificial Sequence Probe
cagctggtcacagggcccacttct

SEQ ID NO: 36 - DNA, 21 bp, Artificial Sequence
  Description of Artificial Sequence Primer
cgatgcttgctggaggataga

SEQ ID NO: 37 - DNA, 22 bp, Artificial Sequence
  Description of Artificial Sequence Primer
acactggtcgtccacactcact

SEQ ID NO: 38 - DNA, 25 bp, Artificial Sequence
  Description of Artificial Sequence Probe
aaagttcccaaagtttgccggctgc

SEQ ID NO: 39 - DNA, 20 bp, Artificial Sequence
  Description of Artificial Sequence Primer
actggtggtggcagatgaca

SEQ ID NO: 40 - DNA, 23 bp, Artificial Sequence
  Description of Artificial Sequence Primer
tcactgttagcaggtagcgcttt

SEQ ID NO: 41 - DNA, 20 bp, Artificial Sequence
  Description of Artificial Sequence Probe
atggccgcattccacggtgc

SEQ ID NO: 42 - DNA, 21 bp, Artificial Sequence
  Description of Artificial Sequence Primer
gaagccatctctgaccggatc

SEQ ID NO: 43 - DNA, 16 bp, Artificial Sequence
  Description of Artificial Sequence Primer
tccgccgacacctcca

SEQ ID NO: 44 - DNA, 22 bp, Artificial Sequence
  Description of Artificial Sequence Probe
ccacaccaatgcgcacgtcagc

SEQ ID NO: 45 - DNA, 20 bp, Artificial Sequence
  Description of Artificial Sequence Primer
gcaggcacaagctgcagata

SEQ ID NO: 46 - DNA, 20 bp, Artificial Sequence
  Description of Artificial Sequence Primer
cctgtggatgcattgattgc

SEQ ID NO: 47 - DNA, 26 bp, Artificial Sequence
  Description of Artificial Sequence Probe
tccattcatccttccgctctctcagc

SEQ ID NO: 48 - DNA, 21 bp, Artificial Sequence
  Description of Artificial Sequence Primer
ccctttcagatcatgttccca

SEQ ID NO: 49 - DNA, 16 bp, Artificial Sequence
  Description of Artificial Sequence Primer
ggacggctgcgacgtc

SEQ ID NO: 50 - DNA, 26 bp, Artificial Sequence
  Description of Artificial Sequence Probe
ccagtacacgatgctgcagtaggcca

SEQ ID NO: 51 - DNA, 21 bp, Artificial Sequence
  Description of Artificial Sequence Primer
actgggccgagacaaatacaa

SEQ ID NO: 52 - DNA, 19 bp, Artificial Sequence
  Description of Artificial Sequence Primer
aatgcgctgcttggtgttg

SEQ ID NO: 53 - DNA, 18 bp, Artificial Sequence
  Description of Artificial Sequence Probe
ccctgcgccagatccggc

SEQ ID NO: 54 - DNA, 22 bp, Artificial Sequence
  Description of Artificial Sequence Primer
ccagctgctactttgacatcga

SEQ ID NO: 55 - DNA, 18 bp, Artificial Sequence
  Description of Artificial Sequence Primer
ccattggacgccctcagt

SEQ ID NO: 56 - DNA, 18 bp, Artificial Sequence
  Description of Artificial Sequence Probe
gatgcgccggtcacgcca

SEQ ID NO: 57 - DNA, 21 bp, Artificial Sequence
  Description of Artificial Sequence Primer
aataaaacagccatgctccca

SEQ ID NO: 58 - DNA, 23 bp, Artificial Sequence
  Description of Artificial Sequence Primer
ccttaagccataagcacttcacc

SEQ ID NO: 59 - DNA, 27 bp, Artificial Sequence
  Description of Artificial Sequence Probe
tgcatgattcgcaggtcagctatttcc

SEQ ID NO: 60 - DNA, 21 bp, Artificial Sequence
  Description of Artificial Sequence Primer
aagcagcttcctgatgcattc

SEQ ID NO: 61 - DNA, 19 bp, Artificial Sequence
  Description of Artificial Sequence Primer
cggacacagcgccttacat

SEQ ID NO: 62 - DNA, 22 bp, Artificial Sequence
  Description of Artificial Sequence Probe
tcgcagccaagaacagccacca

SEQ ID NO: 63 - DNA, 21 bp, Artificial Sequence
  Description of Artificial Sequence Primer
cccagatctccgacacatctg

SEQ ID NO: 64 - DNA, 17 bp, Artificial Sequence
  Description of Artificial Sequence Primer
gcgatgatgccgtccag

SEQ ID NO: 65 - DNA, 24 bp, Artificial Sequence
  Description of Artificial Sequence Probe
ccatggacaacagtcgctccctgg

SEQ ID NO: 66 - DNA, 21 bp, Artificial Sequence
  Description of Artificial Sequence Primer
gctgacaggcaggtgtttgaa

SEQ ID NO: 67 - DNA, 21 bp, Artificial Sequence
  Description of Artificial Sequence Primer
cgaagtagcctgctttgcact

SEQ ID NO: 68 - DNA, 29 bp, Artificial Sequence
  Description of Artificial Sequence Probe
tgtatccacaacacagccggcatctactg

SEQ ID NO: 69 - DNA, 29 bp, Artificial Sequence
  Description of Artificial Sequence Primer
aaaatcttagaacttttgttgggaaacta

SEQ ID NO: 70 - DNA, 21 bp, Artificial Sequence
  Description of Artificial Sequence Primer
ccttgacagttggagaagcca

SEQ ID NO: 71 - DNA, 31 bp, Artificial Sequence
  Description of Artificial Sequence Probe
aaataattggtcctttcccatcagttctgca

SEQ ID NO: 72 - protein, 172 aa, Homo sapiens
MVGPAPRRRLRPLAALALVLALAPGLPTARAGQTPRPAERGPPVRLFTEEELARTGGEEEDQPIYLAVKGVVFDVTSGKEFYGRGAPYNALTGKDSTRGVAKMSLDPADLTHDTTGLTAKELEALDEVFTKVYKAKYPIVGYTARRILNEDGSPNLDFKPEDQPHFDIKDEF

SEQ ID NO: 73 - protein, 101 aa, Murine sp.
GAGCGPSALSLGWADAAPRRARPPVRLFTEEELARYGGEEEDQPIYLAVEGVVFDVTSGKEFYGRGAPYNALAGKDSSRGVAEMSLDPADLTHDTTGLTAK

SEQ ID NO: 74 - protein, 109 aa, Rattus sp.
RPLAALALALALVRVPSARAGQMPRPAERGPPVRLFTEEELARYSGEEEDQPIYLAVKGVVFDVTSGKEFYGRGAPYNALAGKDSSRGVAKMSLDPADLTHDISGLTAK

SEQ ID NO: 75 - DNA, 3900 bp, Homo sapiens
cagtttgcaaaagccagaggtgcaagaagcagcgactgcagcagcagcagcagcagcggcggtggcagcagcagcagcagcggcggcagcagcagcagcagcggaggcaccggtggcagcagcagcatcaccagcaacaacaacaaaaaaaaatcctcatcaaatcctcacctaagctttcagtgtatccagatccacatcttcactcaagccaggagagggaaagaggaaaggggggcaggaaaaaaaaaaaacccaacaacttagcggaaacttctcagagaatgctccaaaactcagcagtgcttctggtgctggtgatcagtgcttctgcaacccatgaggcggagcagaatgactctgtgagccccaggaaatcccgagtggcggctcaaaactcagctgaagtggttcgttgcctcaacagtgctctacaggtcggctgcggggcttttgcatgcctggaaaactccacctgtgacacagatgggatgtatgacatctgtaaatccttcttgtacagcgctgctaaatttgacactcagggaaaagcattcgtcaaagagagcttaaaatgcatcgccaacggggtcacctccaaggtcttcctcgccattcggaggtgctccactttccaaaggatgattgctgaggtgcaggaagagtgctacagcaagctgaatgtgtgcagcatcgccaagcggaaccctgaagccatcactgaggtcgtccagctgcccaatcacttctccaacagatactataacagacttgtccgaagcctgctggaatgtgatgaagacacagtcagcacaatcagagacagcctgatggagaaaattgggcctaacatggccagcctcttccacatcctgcagacagaccactgtgcccaaacacacccacgagctgacttcaacaggagacgcaccaatgagccgcagaagctgaaagtcctcctcaggaacctccgaggtgaggaggactctccctcccacatcaaacgcacatcccatgagagtgcataaccagggagaggttattcacaacctcaccaaactagtatcattttaggggtgttgacacaccaattttgagtgtactgtgcctggtttgatttttttaaagtagttcctattttctatcccccttaaagaaaattgcatgaaactaggcttctgtaatcaatatcccaacattctgcaatggcagcattcccaccaacaaaatccatgtgatcattctgcctctcctcaggagaaagtaccctcttttaccaacttcctctgccatgtcttttcccctgctcccctgagaccacccccaaacacaaaacattcatgtaactctccagccattgtaatttgaagatgtggatccctttagaacggttgccccagtagagttagctgataaggaaactttatttaaatgcatgtcttaaatgctcataaagatgttaaatggaattcgtgttatgaatctgtgctggccatggacgaatatgaatgtcacatttgaattcttgatctctaatgagctagtgtcttatggtcttgatcctccaatgtctaattttctttccgacacatttaccaaattgcttgagcctggctgtccaaccagactttgagcctgcatcttcttgcatctaatgaaaaacaaaaagctaacatctttacgtactgtaactgctcagagctttaaaagtatctttaacaattgtcttaaaaccagagaatcttaaggtctaactgtggaatataaatagctgaaaactaatgtactgtacataaattccagaggactctgcttaaacaaagcagtatataataactttattgcatatagatttagttttgtaacttagctttatttttcttttcctgggaatggaataactatctcacttccagatatccacataaatgctccttgtggccttttttataactaagggggtagaagtagttttaattcaacatcaaaacttaagatgggcctgtatgagacaggaaaaaccaacaggtttatctgaaggaccccaggtaagatgttaatctcccagcccacctcaacccagaggctactcttgacttagacctatactgaaagatctctgtcacatccaactggaaattccaggaaccaaaaagagcatccctatgggcttggaccacttacagtgtgataaggcctactatacattaggaagtggtagttctttactcgtcccctttcatcggtgcctggtactctggcaaatgatgatggggtgggagactttccattaaatcaatcaggaatgagtcaatcagcctttaggtctttagtccgggggacttggggctgagagagtataaataaccctgggctgtccagccttaatagacttctcttacattttcgtcctgtagcacgctgcctgccaaagtagtcctggcagctggaccatctctgtaggatcgtaaaaaaatagaaaaaaagaaaaaaaaaagaaagaaagagggaaaaagagctggtggtttgatcatttctgccatgatgtttacaagatggcgaccaccaaagtcaaacgactaacctatctatgaacaacagtagtttctcagggtcactgtccttgaacccaacagtcccttatgagcgtcactgcccaccaaaggtcaatgtcaagagaggaagagagggaggaggggtaggactgcaggggccactccaaactcgcttaggtagaaactattggtgctcgactctcactaggctaaactcaagatttgaccaaatcgagtgatagggatcctggtgggaggagagagggcacatctccagaaaaatgaaaagcaatacaactttaccataaagcctttaaaaccagtaacgtgctgctcaaggaccaagagcaattgcagcagacccagcagcagcagcagcagcacaaacattgctgcctttgtccccacacagcctctaagcgtgctgacatcagattgttaagggcatttttatactcagaactgtcccatccccaggtccccaaacttatggacactgccttagcctcttggaaatcaggtagaccatattctaagttagactcttcccctccctcccacacttcccacccccaggcaaggctgacttctctgaatcagaaaagctattaaagtttgtgtgttgtgtccattttgcaaacccaactaagccaggaccccaatgcgacaagtagttcatgagtattcctagcaaatttctctctttcttcagttcagtagatttccttttttcttttcttttttttttttttttttttttggctgtgacctcttcaaaccgtggtacccccccttttctccccacgatgatatctatatatgtatctacaatacatatatctacacatacagaaagaagcagttctcacatgttgctagttttttgcttctctttcccccaccctactccctccaattccccccttaaacttccaaagcttcgtcttgtgtttgctgcagagtgattcgggggctgacctagaccagtttgcatgattcttctcttgtgatttggttgcactttagacatttttgtgccattatatttgcattatgtatttataatttaaatgatatttaggtttttggctgagtactggaataaacagtgagcatatctggtatatgtcattatttatgttaaattacattttttaagctccatgtgcatataaaggttatgaaacatatcatggtaatgacagatgcaagttattttatttgcttattttttataattaaagatgccatagcataatatgaagcctttggtgaattccttctaagataaaaataataataaagtgttacgttttattggtttcaaaaaaaaaaaaaaaaaaaa

SEQ ID NO: 76 - protein, 247 aa, Homo sapiens
MLQNSAVLLVLVISASATHEAEQNDSVSPRKSRVAAQNSAEVVRCLNSALQVGCGAFACLENSTCDTDGMYDICKSFLYSAAKFDTQGKAFVKESLKCIANGVTSKVFLAIRRCSTFQRMIAEVQEECYSKLNVCSIAKRNPEAITEVVQLPNHFSNRYYNRLVRSLLECDEDTVSTIRDSLMEKIGPNMASLFHILQTDHCAQTHPRADFNRRRTNEPQKLKVLLRNLRGEEDSPSHIKRTSHESA